(19) United States
(12) Patent Application Publication
(10) Pub. No.: US 2010/0093747 A1
(43) Pub. Date: Apr. 15, 2010

(54) METHOD OF INDUCING NEGATIVE CHEMOTAXIS

(76) Inventor: Erica Brook Goodhew, Atlanta, GA (US)

Correspondence Address: ELMORE PATENT LAW GROUP, PC, 515 Groton Road, Unit 1R, Westford, MA, 01886, US

(21) Appl. No.: 12/576,421

(22) Filed: Oct. 9, 2009

Related U.S. Application Data
(60) Provisional application No. 61/104,551, filed on Oct. 10, 2008.

(51) Int. Cl. A61K 31/4184; A61K 31/427; A61K 31/422; A61K 31/506; A61K 31/428; A61K 31/501; A61K 31/4245; A61K 31/437; A61P 29/00

(52) U.S. Cl. 514/252.06; 514/371; 514/380; 514/256; 514/394; 514/367; 514/364; 514/300; 514/275

(57) ABSTRACT

The current invention is directed to methods of inducing the negative chemotaxis of a migratory cell comprising contacting the cell with a compound having the Formula (I), (II), (III) or (IV).

### RELATED APPLICATION

This application claims the benefit of U.S. Provisional Application No. 61/104,551, filed Oct. 10, 2008. The entire teachings of the above application are incorporated herein by reference.

### BACKGROUND OF THE INVENTION

Chemotaxis, or the oriented movement of a cell in response to a chemical agent, is a complex and highly integrated process. The movement can be positive (toward) or negative (away) from a chemical gradient. Movement toward an agent or stimulus is termed positive chemotaxis (i.e., the agent or stimulus is chemoattractive for the cell), while movement away from an agent or stimulus is termed negative chemotaxis (i.e., the agent or stimulus is chemorepulsive for the cell). It is believed that for both prokaryotes and eukaryotes, cells undergoing chemotaxis sense a change in agent concentration and, thereby, move in response to the concentration gradient. Chemoattraction (CA) and chemorepulsion (CR) are therefore properties of the agent or stimulus, while chemotaxis is a property of cells.

Within the immune system, chemotaxis is often driven by a class of biological agents, known as chemokines (or chemotactic cytokines). Once triggered, chemotaxis plays an important role in various physiologic and cellular processes including tissue organization, organogenesis, homeostasis, embryonic morphogenesis tissue repair and regeneration and disease progression in cancer, mental retardation, atherosclerosis, and arthritis. Compounds that affect chemotaxis (either induce positive or negative chemotaxis) would therefore be useful in modulating these and other biologic processes. Compounds that induce negative chemotaxis have in fact been described as useful in treating inflammation, in inhibiting tumor metastasis and in contraception.

It would therefore be advantageous to identify additional compounds that induce negative chemotaxis.

### SUMMARY OF THE INVENTION

The present invention provides methods of inducing the negative chemotaxis of a migratory cell comprising contacting the cell with omeprazole or a derivative thereof.

The present invention provides methods of inducing the negative chemotaxis of a migratory cell comprising contacting the cell with a compound having a structure selected from the group consisting of Formulae (I), (II), (III) and (IV):

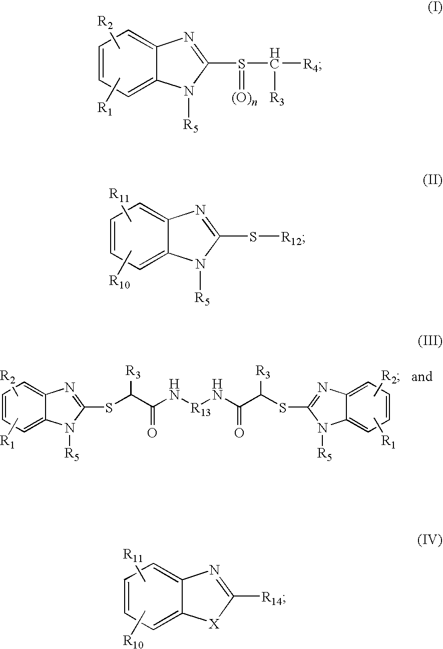

or a pharmaceutically acceptable salt of any of thereof;
wherein:

R₁ and R₂ are the same or different and each are selected from the group consisting of hydrogen, C1-C10 alkyl, halo, OR₉, C(O)R₉, C(O)OR₉, NO₂ and NR₆R_{6′};

R₃ is selected from the group consisting of H, C1-C10 alkyl and aryl;

R₄ is selected from the group consisting of C1-C10 alkyl, C1-C10 alkyl substituted with one or more R₇, C(O)R₆, C(O)OR₆, C(O)NR₆R_{6′}, benzyl, aryl and heteroaryl, wherein said aryl and heteroaryl are each optionally substituted with one or more R₈;

Each R₅ is independently selected from the group consisting of hydrogen, C1-C10 alkyl and S(O)ₚR₆;

R₆ and R₆′ are each independently selected from the group consisting of hydrogen, C1-C10 alkyl, C1-C10 alkyl substituted with one or more R₇, C2-C10 alkeynyl, C2-C10 alkenyl substituted with one or more R₇, halo, OR₉, cycloalkyl, cycloalkyl substituted with one or more R₈ bicycloalkyl, bicycloalkyl substituted with one or more R₈, heterocylic, heterocyclic substituted with one or more R₈, C(O)R₉, OC(O)R₉, C(O)OR₉, benzyl, aryl and heteroaryl, wherein said aryl and heteroaryl are each optionally substituted with one or more R₈, or wherein R₆ and R_{6′} are taken together with the nitrogen to which they are attached to form a 3-6 membered heterocyclic ring, wherein the ring is optionally substituted with one or more R₈;

Each R₇ is independently selected from the group consisting of halo, NR₁₆R_{16′}, NO₂, C(O)R₉, C(O)OR₉, C(O)NR₁₆R_{16′}, OR₉, S(O)ₚR₁₆, S(O)ₚNR₁₆R_{16′}, SR₁₆, CN, oxo, 5-6 membered heterocyclic ring comprising one or more heteroatoms selected from N, O or S, wherein said heterocyclic ring is optionally substituted with R₉, optionally substituted aryl, optionally substituted heteroaryl, and

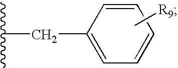

Each R₈ is independently selected from the group consisting of R₇, C1-C10 alkyl and C1-C10 alkyl substituted with one or more R₇;

Each R₉ is independently selected from the group consisting of hydrogen, optionally substituted aryl, optionally substituted heteroaryl, C1-C10 alkyl, C1-C10 alkyl substituted with one or more group selected from halo, optionally substituted aryl and optionally substituted heteroaryl;

R₁₀ and R₁₁ are the same or different and are each independently selected from the group consisting of hydrogen, C1-C10 alkyl, halo, OR₉, C(O)R₉, C(O)OR₉, NO₂ and NR₆R_{6′}, SR₆, aryl, heteroaryl, wherein each of said aryl and heteroaryl is optionally substituted with one or more R₈, and

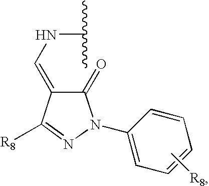

R₁₂ is selected from the group consisting of hydrogen, C1-C10 alkyl, aryl, heteroaryl, wherein said each of said aryl and heteroaryl are optionally substituted by one or more R₈;

R₁₃ is selected from the group consisting of:

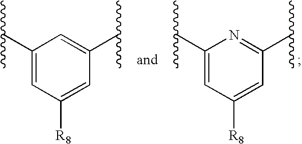

R₁₄ is selected from the group consisting of hydrogen, C1-C10 alkyl, C1-C10 alkyl substituted with one or more R₇, S(O)₂OR₆, NR₆R_{6′}, SR₆, OR₆, aryl, and heteroaryl, wherein each of said aryl and heteroaryl each optionally substituted with one or more R₈, and

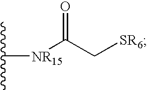

X is selected from the group consisting of O, S and NR₁₅;

R₁₅ is hydrogen or C1-C10 alkyl;

R₁₆ and R₁₆′ are each independently selected from the group consisting of hydrogen, C1-C10 alkyl, C1-C10 alkyl substituted with one or more R₉, C2-C10 alkeynyl, C2-C10 alkenyl substituted with one or more R₉, halo, OR₉, cycloalkyl, cycloalkyl substituted with one or more R₉, bicycloalkyl, bicycloalkyl substituted with one or more R₉, heterocylic, heterocyclic substituted with one or more R₉, C(O)R₉, OC(O)R₉, C(O)OR₉, benzyl, optionally substituted aryl and optionally substituted heteroaryl; or R₁₆ and R_{16′} are taken together with the nitrogen atom to which they are attached to form a 3-6 membered heterocyclic ring, wherein the ring is optionally substituted with one or more R₉;

n is 0 or 1; and

each p is independently 1 or 2.

In another embodiment, the invention is a method of inducing negative chemotaxis of a human immune cell comprising administering a compound having a structure selected from Formula (I), Formula (II), Formula (III) or Formula (IV).

In yet another embodiment, the invention is a method of treating a patient suffering from a condition mediated by migration of a human migratory cell toward a chemotactic site comprising administering to said patient a compound having a structure selected from Formula (I), Formula (II), Formula (III), or Formula (IV) wherein the compound is administered in an amount effective to inhibit migration of the cell toward the chemotactic site.

In a further embodiment, the invention is a method of treating a patient suffering from an inflammatory condition comprising administering to said patient a compound having a structure with a formula selected from Formula (I), (II), (III) or (IV) wherein the compound is administered in a therapeutically effective amount.

In an additional embodiment, the invention is a method of inhibiting angiogenesis in a patient in need thereof comprising administering to said patient a compound having a structure having a formula selected from Formula (I), (II), (III) or (IV) wherein the compound is administered in a therapeutically effective amount.

In yet another embodiment, the invention is a method of contraception comprising administering a compound of Formula (I), (II), (III) or (IV) in a patient in need thereof in an amount effective to inhibit migration of germ cells in the subject.

These and other aspects of the invention, as well as various advantages and utilities, will be more apparent with reference to the drawings and the detailed description of the embodiments of the invention.

### BRIEF DESCRIPTION OF THE DRAWINGS

FIGS. 1A, 1B and 1C are bar graphs showing fold induction of chemotaxis after incubation of neutrophils with 0.048, 0.48, 4.8 or 48 uM esomeprazole relative to induction in the presence of media alone.

FIG. 2A is a bar graph showing fold induction of chemorepulsion of neutrophils incubated with 0.1, 1, 10 and 100 uM omeprazole.

FIG. 2B is a bar graph showing fold induction of chemorepulsion and chemoattraction with 0.1, 1, 10 and 100 uM omeprazole.

FIG. 3A is a bar graph showing fold induction of chemorepulsion of neutrophils incubated with Compound 60 at 0.1, 1, 10 and 100 uM.

FIG. 3B is a bar graph showing fold induction of chemorepulsion (right) and chemoattraction (left) with 0.1, 1, 10 and 100 um Compound 60.

FIG. 4 is a bar graph showing fold induction of chemorepulsion of neutrophils incubated with Compound 79 at 0.1, 1, 10 and 100 uM.

FIG. 5 is a bar graph showing fold induction of chemorepulsion of neutrophils incubated with Compound 80 at 0.1, 1, 10 and 100 uM.

FIG. 6 is a bar graph showing fold induction of chemorepulsion of neutrophils incubated with Compound 81 at 0.1, 1, 10 and 100 uM.

FIG. 7 is a bar graph showing fold induction of chemorepulsion of neutrophils incubated with Compound 109 at 0.1, 1, 10 and 100 uM.

FIG. 8 is a bar graph showing fold induction of chemorepulsion of neutrophils incubated with Compound 110 at 0.1, 1, 10 and 100 uM.

FIG. 9 is a bar graph showing fold induction of chemorepulsion of neutrophils incubated with Compound 111 at 0.1, 1, 10 and 100 uM.

FIG. 10 is a bar graph showing fold induction of chemorepulsion of neutrophils incubated with Compound 112 at 0.1, 1, 10 and 100 uM.

FIG. 11A is a bar graphs showing fold induction of chemorepulsion of neutrophils incubated with Compound 11 at 0.1, 1, 10 and 100 uM.

FIG. 11B is a bar graph showing fold induction of chemorepulsion (right) and chemoattraction (left) with 0.1, 1, 10 and 100 um Compound 11.

FIG. 12A is a bar graph showing fold induction of chemorepulsion of neutrophils incubated with Compound 12 at 0.1, 1, 10 and 100 uM.

FIG. 12B is a bar graph showing fold induction of chemorepulsion (right) and chemoattraction (left) with 0.1, 1, 10 and 100 um Compound 12.

FIG. 13A is a bar graph showing fold induction of chemorepulsion of neutrophils incubated with Compound 15 at 0.1, 1, 10 and 100 uM.

FIG. 13B is a bar graph showing fold induction of chemorepulsion (right) and chemoattraction (left) with 0.1, 1, 10 and 100 um Compound 15.

FIG. 14A is a bar graph showing fold induction of chemorepulsion of neutrophils incubated with Compound 16 at 0.1, 1, 10 and 100 uM.

FIG. 14B is a bar graph showing fold induction of chemorepulsion (right) and chemoattraction (left) with 0.1, 1, 10 and 100 um Compound 16.

FIG. 15A is a bar graph showing fold induction of chemorepulsion of neutrophils incubated with Compound 17 at 0.1, 1, 10 and 100 uM.

FIG. 15B is a bar graph showing fold induction of chemorepulsion (right) and chemoattraction (left) with 0.1, 1, 10 and 100 um Compound 17.

FIG. 16A is a bar graph showing fold induction of chemorepulsion of neutrophils incubated with Compound 20 at 0.1, 1, 10 and 100 uM.

FIG. 16B is a bar graph showing fold induction of chemorepulsion (right) and chemoattraction (left) with 0.1, 1, 10 and 100 um Compound 20.

FIG. 17A is a bar graph showing fold induction of chemorepulsion of neutrophils incubated with Compound 21 at 0.1, 1, 10 and 100 uM.

FIG. 17B is a bar graph showing fold induction of chemorepulsion (right) and chemoattraction (left) with 0.1, 1, 10 and 100 um Compound 21.

FIG. 18A is a bar graph showing fold induction of chemorepulsion of neutrophils incubated with Compound 22 at 0.1, 1, 10 and 100 uM.

FIG. 18B is a bar graph showing fold induction of chemorepulsion (right) and chemoattraction (left) with 0.1, 1, 10 and 100 um Compound 22.

FIG. 19A is a bar graph showing fold induction of chemorepulsion of neutrophils incubated with Compound 23 at 0.1, 1, 10 and 100 uM.

FIG. 19B is a bar graph showing fold induction of chemorepulsion (right) and chemoattraction (left) with 0.1, 1, 10 and 100 um Compound 23.

FIG. 20A is a bar graph showing fold induction of chemorepulsion of neutrophils incubated with Compound 24 at 0.1, 1, 10 and 100 uM.

FIG. 20B is a bar graph showing fold induction of chemorepulsion (right) and chemoattraction (left) with 0.1, 1, 10 and 100 um Compound 24.

FIG. 21A is a bar graph showing fold induction of chemorepulsion of neutrophils incubated with Compound 33 at 0.1, 1, 10 and 100 uM.

FIG. 21B is a bar graph showing fold induction of chemorepulsion (right) and chemoattraction (left) with 0.1, 1, 10 and 100 um Compound 33.

FIG. 22A is a bar graph showing fold induction of chemorepulsion of neutrophils incubated with Compound 40 at 0.1, 1, 10 and 100 uM.

FIG. 22B is a bar graph showing fold induction of chemorepulsion (right) and chemoattraction (left) with 0.1, 1, 10 and 100 um Compound 40.

FIG. 23A is a bar graph showing fold induction of chemorepulsion of neutrophils incubated with Compound 41 at 0.1, 1, 10 and 100 uM.

FIG. 23B is a bar graph showing fold induction of chemorepulsion (right) and chemoattraction (left) with 0.1, 1, 10 and 100 um Compound 41.

FIG. 24A is a bar graph showing fold induction of chemorepulsion of neutrophils incubated with Compound 42 at 0.1, 1, 10 and 100 uM.

FIG. 24B is a bar graph showing fold induction of chemorepulsion (right) and chemoattraction (left) with 0.1, 1, 10 and 100 um Compound 42.

FIG. 25A is a bar graph showing fold induction of chemorepulsion of neutrophils incubated with Compound 43 at 0.1, 1, 10 and 100 uM.

FIG. 25B is a bar graph showing fold induction of chemorepulsion (right) and chemoattraction (left) with 0.1, 1, 10 and 100 um Compound 43.

FIG. 26A is a bar graph showing fold induction of chemorepulsion of neutrophils incubated with Compound 45 at 0.1, 1, 10 and 100 uM.

FIG. 26B is a bar graph showing fold induction of chemorepulsion (right) and chemoattraction (left) with 0.1, 1, 10 and 100 um Compound 45.

FIG. 27A is a bar graph showing fold induction of chemorepulsion of neutrophils incubated with Compound 54 at 0.1, 1, 10 and 100 uM.

FIG. 27B is a bar graph showing fold induction of chemorepulsion (right) and chemoattraction (left) with 0.1, 1, 10 and 100 um Compound 54.

FIG. 28A is a bar graph showing fold induction of chemorepulsion of neutrophils incubated with Compound 56 at 0.1, 1, 10 and 100 uM.

FIG. 28B is a bar graph showing fold induction of chemorepulsion (right) and chemoattraction (left) with 0.1, 1, 10 and 100 um Compound 56.

FIG. 29A is a bar graph showing fold induction of chemorepulsion of neutrophils incubated with Compound 58 at 0.1, 1, 10 and 100 uM.

FIG. 29B is a bar graph showing fold induction of chemorepulsion (right) and chemoattraction (left) with 0.1, 1, 10 and 100 um Compound 58.

FIG. 30A is a bar graph showing fold induction of chemorepulsion of neutrophils incubated with Compound 59 at 0.1, 1, 10 and 100 uM.

FIG. 30B is a bar graph showing fold induction of chemorepulsion (right) and chemoattraction (left) with 0.1, 1, 10 and 100 um Compound 59.

FIG. 31A is a bar graph showing fold induction of chemorepulsion of neutrophils incubated with Compound 62 at 0.1, 1, 10 and 100 uM.

FIG. 31B is a bar graph showing fold induction of chemorepulsion (right) and chemoattraction (left) with 0.1, 1, 10 and 100 um Compound 62.

FIG. 32A is a bar graph showing fold induction of chemorepulsion of neutrophils incubated with Compound 63 at 0.1, 1, 10 and 100 uM.

FIG. 32B is a bar graph showing fold induction of chemorepulsion (right) and chemoattraction (left) with 0.1, 1, 10 and 100 um Compound 63.

FIG. 33 is a bar graph showing fold induction of chemorepulsion of neutrophils incubated with Compound 69 at 0.1, 1, 10 and 100 uM.

FIG. 34 is a bar graph showing fold induction of chemorepulsion of neutrophils incubated with Compound 70 at 0.1, 1, 10 and 100 uM.

FIG. 35 is a bar graph showing fold induction of chemorepulsion of neutrophils incubated with Compound 71 at 0.1, 1, 10 and 100 uM.

FIG. 36 is a bar graph showing fold induction of chemorepulsion of neutrophils incubated with Compound 83 at 0.1, 1, 10 and 100 uM.

FIG. 37 is a bar graph showing fold induction of chemorepulsion of neutrophils incubated with Compound 84 at 0.1, 1, 10 and 100 uM.

FIG. 38 is a bar graph showing fold induction of chemorepulsion of neutrophils incubated with Compound 88 at 0.1, 1, 10 and 100 uM.

FIG. 39 is a bar graph showing fold induction of chemorepulsion of neutrophils incubated with Compound 89 at 0.1, 1, 10 and 100 uM.

FIG. 40 is a bar graph showing fold induction of chemorepulsion of neutrophils incubated with Compound 91 at 0.1, 1, 10 and 100 uM.

FIG. 41 is a bar graph showing fold induction of chemorepulsion of neutrophils incubated with Compound 92 at 0.1, 1, 10 and 100 uM.

FIG. 42 is a bar graph showing fold induction of chemorepulsion of neutrophils incubated with Compound 93 at 0.1, 1, 10 and 100 uM.

FIG. 43 is a bar graph showing fold induction of chemorepulsion of neutrophils incubated with Compound 96 at 0.1, 1, 10 and 100 uM.

FIG. 44 is a bar graph showing fold induction of chemorepulsion of neutrophils incubated with Compound 97 at 0.1, 1, 10 and 100 uM.

FIG. 45 is a bar graph showing fold induction of chemorepulsion of neutrophils incubated with Compound 98 at 0.1, 1, 10 and 100 uM.

FIG. 46 is a bar graph showing fold induction of chemorepulsion of neutrophils incubated with Compound 99 at 0.1, 1, 10 and 100 uM.

FIG. 47 is a bar graph showing fold induction of chemorepulsion of neutrophils incubated with Compound 100 at 0.1, 1, 10 and 100 uM.

FIG. 48 is a bar graph showing fold induction of chemorepulsion of neutrophils incubated with Compound 101 at 0.1, 1, 10 and 100 uM.

FIG. 49 is a bar graph showing fold induction of chemorepulsion of neutrophils incubated with Compound 107 at 0.1, 1, 10 and 100 uM.

FIG. 50 is a bar graph showing fold induction of chemorepulsion of neutrophils incubated with Compound 128 at 0.1, 1, 10 and 100 uM.

FIG. 51 is a bar graph showing fold induction of chemorepulsion of neutrophils incubated with Compound 133 at 0.1, 1, 10 and 100 uM.

FIG. 52 is a bar graph showing fold induction of chemorepulsion of neutrophils incubated with Compound 134 at 0.1, 1, 10 and 100 uM.

FIG. 53 is a bar graph showing fold induction of chemorepulsion of neutrophils incubated with Compound 136 at 0.1, 1, 10 and 100 uM.

FIG. 54 is a bar graph showing fold induction of chemorepulsion of neutrophils incubated with Compound 146 at 0.1, 1, 10 and 100 uM.

FIG. 55 is a bar graph showing fold induction of chemorepulsion of neutrophils incubated with Compound 143 at 0.1, 1, 10 and 100 uM.

FIG. 56 is a bar graph showing fold induction of chemorepulsion of neutrophils incubated with Compound 144 at 0.1, 1, 10 and 100 uM.

FIG. 57 is a bar graph showing fold induction of chemorepulsion of neutrophils incubated with Compound 132 at 0.1, 1, 10 and 100 uM.

FIG. 58 is a bar graph showing fold induction of chemorepulsion of neutrophils incubated with Compound 135 at 0.1, 1, 10 and 100 uM.

FIG. 59 is a bar graph showing fold induction of chemorepulsion of neutrophils incubated with rabeprazole at 0.106, 1.06, 10.6 and 106 uM.

FIG. 60 is a bar graph showing fold induction of chemorepulsion of neutrophils incubated with Compound 5 at 0.1, 1, 10 and 100 uM.

FIG. 61 is a bar graph showing fold induction of chemorepulsion of neutrophils incubated with Compound 6 at 0.1, 1, 10 and 100 uM.

FIG. 62 is a bar graph showing fold induction of chemorepulsion of neutrophils incubated with Compound 19 at 0.1, 1, 10 and 100 uM.

FIG. 63 is a bar graph showing fold induction of chemorepulsion of neutrophils incubated with Compound 32 at 0.1, 1, 10 and 100 uM.

FIG. 64 is a bar graph showing fold induction of chemorepulsion (right) and chemoattraction (left) with 0.1, 1, 10 and 100 um Compound 36.

FIG. 65 is a bar graph showing fold induction of chemorepulsion (right) and chemoattraction (left) with 0.1, 1, 10 and 100 um Compound 37.

FIG. 66 is a bar graph showing fold induction of chemorepulsion (right) and chemoattraction (left) with 0.1, 1, 10 and 100 um Compound 38.

FIG. 67 is a bar graph showing fold induction of chemorepulsion (right) and chemoattraction (left) with 0.1, 1, 10 and 100 um Compound 39.

FIG. 68 is a bar graph showing fold induction of chemorepulsion (right) and chemoattraction (left) with 0.1, 1, 10 and 100 um Compound 46.

FIG. 69 is a bar graph showing fold induction of chemorepulsion of neutrophils incubated with Compound 48 at 0.1, 1, 10 and 100 uM.

FIG. 70 is a bar graph showing fold induction of chemorepulsion of neutrophils incubated with Compound 49 at 0.1, 1, 10 and 100 uM.

FIG. 71 is a bar graph showing fold induction of chemorepulsion of neutrophils incubated with Compound 53 at 0.1, 1, 10 and 100 uM.

FIG. 72 is a bar graph showing fold induction of chemorepulsion of neutrophils incubated with Compound 55 at 0.1, 1, 10 and 100 uM.

FIG. 73 is a bar graph showing fold induction of chemorepulsion of neutrophils incubated with Compound 64 at 0.1, 1, 10 and 100 uM.

FIG. 74 is a bar graph showing fold induction of chemorepulsion of neutrophils incubated with Compound 65 at 0.1, 1, 10 and 100 uM.

FIG. 75 is a bar graph showing fold induction of chemorepulsion of neutrophils incubated with Compound 67 at 0.1, 1, 10 and 100 uM.

FIG. 76 is a bar graph showing fold induction of chemorepulsion of neutrophils incubated with Compound 68 at 0.1, 1, 10 and 100 uM.

FIG. 77 is a bar graph showing fold induction of chemorepulsion of neutrophils incubated with Compound 73 at 0.1, 1, 10 and 100 uM.

FIG. 78 is a bar graph showing fold induction of chemorepulsion of neutrophils incubated with Compound 75 at 0.1, 1, 10 and 100 uM.

FIG. 79 is a bar graph showing fold induction of chemorepulsion of neutrophils incubated with Compound 76 at 0.1, 1, 10 and 100 uM.

FIG. 80 is a bar graph showing fold induction of chemorepulsion of neutrophils incubated with Compound 77 at 0.1, 1, 10 and 100 uM.

FIG. 81 is a bar graph showing fold induction of chemorepulsion of neutrophils incubated with Compound 82 at 0.1, 1, 10 and 100 uM.

FIG. 82 is a bar graph showing fold induction of chemorepulsion of neutrophils incubated with Compound 85 at 0.1, 1, 10 and 100 uM.

FIG. 83 is a bar graph showing fold induction of chemorepulsion of neutrophils incubated with Compound 94 at 0.1, 1, 10 and 100 uM.

FIG. 84 is a bar graph showing fold induction of chemorepulsion of neutrophils incubated with Compound 95 at 0.1, 1, 10 and 100 uM.

FIG. 85 is a bar graph showing fold induction of chemorepulsion of neutrophils incubated with Compound 102 at 0.1, 1, 10 and 100 uM.

FIG. 86 is a bar graph showing fold induction of chemorepulsion of neutrophils incubated with Compound 106 at 0.1, 1, 10 and 100 uM.

FIG. 87 is a bar graph showing fold induction of chemorepulsion of neutrophils incubated with Compound 108 at 0.1, 1, 10 and 100 uM.

FIG. 88 is a bar graph showing fold induction of chemorepulsion of neutrophils incubated with Compound 113 at 0.1, 1, 10 and 100 uM.

FIG. 89 is a bar graph showing fold induction of chemorepulsion of neutrophils incubated with Compound 114 at 0.1, 1, 10 and 100 uM.

FIG. 90 is a bar graph showing fold induction of chemorepulsion of neutrophils incubated with Compound 118 at 0.1, 1, 10 and 100 uM.

FIG. 91 is a bar graph showing fold induction of chemorepulsion of neutrophils incubated with Compound 121 at 0.1, 1, 10 and 100 uM.

FIG. 92 is a bar graph showing fold induction of chemorepulsion of neutrophils incubated with Compound 125 at 0.1, 1, 10 and 100 uM.

FIG. 93 is a bar graph showing fold induction of chemorepulsion of neutrophils incubated with Compound 137 at 0.1, 1, 10 and 100 uM.

FIG. 94 is a bar graph showing fold induction of chemorepulsion of neutrophils incubated with Compound 138 at 0.1, 1, 10 and 100 uM.

FIG. 95 is a bar graph showing fold induction of chemorepulsion of neutrophils incubated with Compound 139 at 0.1, 1, 10 and 100 uM.

FIG. 96 is a bar graph showing fold induction of chemorepulsion of neutrophils incubated with Compound 141 at 0.1, 1, 10 and 100 uM.

FIG. 97 is a bar graph showing fold induction of chemorepulsion of neutrophils incubated with Compound 146 at 0.1, 1, 10 and 100 uM.

FIG. 98 is a bar graph showing fold induction of chemorepulsion of neutrophils incubated with Compound 149 at 0.1, 1, 10 and 100 uM.

FIG. 99 is a bar graph showing fold induction of chemorepulsion of neutrophils incubated with Compound 151 at 0.1, 1, 10 and 100 uM.

FIG. 100 is a bar graph showing fold induction of chemorepulsion of neutrophils incubated with Compound 152 at 0.1, 1, 10 and 100 uM.

FIG. 101 is a bar graph showing fold induction of chemorepulsion of neutrophils incubated with Compound 153 at 0.1, 1, 10 and 100 uM.

FIG. 102 is a bar graph showing fold induction of chemorepulsion of neutrophils incubated with Compound 154 at 0.1, 1, 10 and 100 uM.

FIG. 103 is a bar graph showing fold induction of chemorepulsion of neutrophils incubated with Compound 156 at 0.1, 1, 10 and 100 uM.

FIG. 104 is a bar graph showing fold induction of chemorepulsion of neutrophils incubated with Compound 157 at 0.1, 1, 10 and 100 uM.

FIG. 105 is a bar graph showing fold induction of chemorepulsion of neutrophils incubated with Compound 159 at 0.1, 1, 10 and 100 uM.

FIG. 106 is a bar graph showing fold induction of chemorepulsion of neutrophils incubated with Compound 160 at 0.1, 1, 10 and 100 uM.

FIG. 107 is a bar graph showing fold induction of chemorepulsion of neutrophils incubated with Compound 64 at 0.1, 1, 10 and 100 uM.

### DETAILED DESCRIPTION OF THE INVENTION

A description of the embodiments of the invention follows.

As used herein, “a” or “an” are taken to mean one or more unless otherwise specified.

The present invention is based on the surprising discovery that omeprazole and omeprazole derivatives compounds induce negative chemotaxis of human migratory cells. For example, as shown in Example 2, neutrophils contacted with 48 uM esomeprazole showed between about 10 to about 20-fold greater induction of negative chemotaxis compared to media.

Esomeprazole and omeprazole belong to the substituted 2-(2-pyridinylmethylsuflinyl)-1H-benzimadazole class of compounds. Substituted 2-(2-pyridinylmethylsuflinyl)-1H-benzimadazoles have previously been described as proton pump inhibitors with utility in inhibiting gastric acid secretion. These compounds have been described extensively in the literature, including in U.S. Pat. Nos. 4,255,431, 4,337,257, 4,628,098, 4,689,333, 4,628,098, 4,738,974, 4,758,579, 4,786,505, 4,853,230, 5,013,743, 5,026,560, 5,035,899, 5,045,321, 5,045,552, 5,093,132, 5,433,959, 5,464,632, 5,690,060, 5,714,504, 5,877,192, 5,900,424, 5,997,903, 6,166,213, 6,191,148, 6,328,994, 6,369,085, 6,428,810, 6,780,881, 6,875,872 and 7,351,723, the contents of which are incorporated by reference herein.

C₁₋₄ alkyl radicals include, for example, methyl, ethyl, propyl, isopropyl, butyl and tert-butyl.

C₁₋₃ alkoxy radicals include, for example, methoxy, ethoxy, propoxy and isopropoxy.

C₁₋₃ alkoxy radicals which are completely or predominantly substituted by fluorine contain in addition to the oxygen atom, the C₁₋₃ alkyl radicals which are completely or predominantly substituted by fluorine. Examples include the 1,1,2,2-tetrafluoroethoxy, the trifluoromethoxy, the 2,2,2-trifluoroethoxy and the difluoromethoxy radicals.

Examples of C₁₋₂-alkylenedioxy radicals which optionally, completely or partly substituted with fluorine are the 1,1-difluoroethylenedioxy radical, the 1,1,2,2-tetrafluoroethylenedioxy radical, the 1,1,1-trifluoroethylenedioxy radical and in particular, the difluoromethylenedioxy radical, as substituted radicals, and the ethylenedioxy radical and the methylenedioxy radical.

The term “alkyl”, as used herein, unless otherwise indicated, refers to both branched and straight-chain saturated aliphatic hydrocarbon groups having the specified number of carbon atoms; for example, “C1-C10 alkyl” denotes alkyl having 1 to 10 carbon atoms. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term, “alkenyl”, as used herein, refers to both straight and branched-chain moieties having the specified number of carbon atoms and having at least one carbon-carbon double bond. The term, “alkynyl”, as used herein, refers to both straight and branched-chain moieties having the specified number or carbon atoms and having at least one carbon-carbon triple bond.

The term “cycloalkyl,” as used herein, refers to cyclic alkyl moieties having 3 or more carbon atoms. Examples of cycloalkyl include, but are not limited to, cyclopropyl, cyclobutyl, cyclopentyl, cyclohexyl, and cycloheptyl.

The term “bicycloalkyl,” as used herein, refers to a non-aromatic saturated carbocyclic group consisting of two rings.

The term “heterocyclic” or as used herein refers to a cycloalkyl, cycloalkenyl, bicycloalkyl, bicycloalkenyl, tricycloalkyl, tricycloalkenyl groups containing one or more heteroatoms (O, S, or N) within the ring. The term “heterocyclic” encompasses heterocycloalkyl, heterocycloalkenyl, heterobicycloalkenyl, heterobicycloalkyl, heterobicyclalkyl and the like.

Cycloalkyl, cycloalkenyl, bicycloalkyl, bicycloalkenyl, heterocycloalkyl, heterocycloalkenyl, heterobicycloalkyl and heterobicycloalkenyl groups also include groups similar to those described above for each of these respective categories, but which are substituted with one or more oxo moieties and/or are fused to one or more aromatic rings.

The term “aryl”, as used herein, refers to an aromatic carbocyclic group containing one or more rings wherein such rings may be attached together in a fused manner. The term “aryl” embraces aromatic radicals, such as, phenyl, naphthyl, indenyl, tetrahydronaphthyl, and indanyl. An aryl group may be substituted or unsubstituted.

A suitable substituent on an aryl is any substituent that does not substantially interfere with the pharmaceutical activity of the disclosed compound. An aryl may have one or more substituents, which can be identical or different. Examples of suitable substituents for a substitutable carbon atom in an aryl group include, but are not limited to, optionally substituted alkyl, optionally substituted alkenyl, optionally substituted alkynyl, optionally substituted cycloalkyl and cycloalkenyl, optionally substituted heterocycloalkyl and heterocycloalkenyl, halo, alkoxy, nitro, amino, carboxy, aminosulfonyl, aminocarbonyl, sulfinyl, sulfanyl, sulfonyl, hydroxy, alkoxycarbonyl, carbamate, trihalomethyl, cyano, mercapto, optionally substituted aryl and optionally substituted heteroaryl, oxo, thioxo, —NH-alkyl, —NH-alkenyl, —NH—C₃-C₁₂-cycloalkyl, —NH-aryl, —NH-heteroaryl, —NH-heterocyclic, -dialkylamino, —O—C₁-C₁₂-alkyl, —O—C₂-C₈-alkenyl, —O-cycloalkyl, —O-aryl, —O-heteroaryl, —O-heterocyciclic, —C(O)-alkyl, —C(O)-alkenyl, —C(O)-alkynyl, —C(O)-cycloalkyl, —C(O)-aryl, —C(O)-heteroaryl, —C(O)-heterocycloalkyl, —CONH₂, —CONH—-alkyl, —CONH—-alkenyl, —CONH-alkynyl, —CONH-cycloalkyl, —CONH-aryl, —CONH-heteroaryl, —CONH-heterocyclic, —O-alkyl, —OCO₂-alkenyl, —OCO₂-alkynyl, —OCO₂-cycloalkyl, —OCO₂-aryl, —OCO₂-heteroaryl, —OCO₂-heterocycloalkyl, —CO₂-alkyl, —CO₂-alkenyl, —CO₂-alkynyl, CO₂-cycloalkyl, —CO₂-aryl, CO₂-heteroaryl, CO₂-heterocyloalkyl, —OCONH₂, —OCONH-alkyl, —OCONH-alkenyl, —OCONH-alkynyl, —OCONH-cycloalkyl, —OCONH-aryl, —OCONH-heteroaryl, —OCONH-heterocyclic, —NHC(O)H, —NHC(O)-alkyl, —NHC(O)-alkenyl, —NHC(O)-alkynyl, —NHC(O)-cycloalkyl, —NHC(O)-aryl, —NHC(O)-heteroaryl, —NHC(O)-heterocyclic, —NHCO₂—-alkyl, —NHCO₂-alkenyl, —NHCO₂—-alkynyl, —NHCO₂-cycloalkyl, —NHCO₂-aryl, —NHCO₂-heteroaryl, —NHCO₂-heterocyclic, —NHC(O)NH₂, —NHC(O)NH-alkyl, —NHC(O)NH-alkenyl, —NHC(O)NH—-alkynyl, —NHC(O)NH-cycloalkyl, —NHC(O)NH-aryl, —NHC(O)NH-heteroaryl, —NHC(O)NH-heterocyclic, —S(O)-alkyl, —S(O)—-alkenyl, —S(O)—-alkynyl, —S(O)—-cycloalkyl, —S(O)-aryl, —S(O)-heteroaryl, —S(O)-heterocyclic, —SO₂NH₂, —SO₂NH-alkyl, —SO₂NH-alkenyl, —SO₂NH—-alkynyl, —SO₂NH—-cycloalkyl, —SO₂NH-aryl, —SO₂NH-heteroaryl, —SO₂NH-heterocyclic, —NHSO₂—-alkyl, —NHSO₂-alkenyl, —NHSO₂-alkynyl, —NHSO₂-cycloalkyl, —NHSO₂-aryl, —NHSO₂-heteroaryl, —NHSO₂-heterocycloalkyl, —CH₂NH₂, —CH₂SO₂CH₃, -arylalkyl, -heteroarylalkyl, -heterocycloalkyl, polyalkoxyalkyl, —SH, —S-alkyl, —S-alkenyl, —S-alkynyl, —S-cycloalkyl, —S-aryl, —S-heteroaryl, —S-heterocycloalkyl, and the like. Non-limiting examples of optionally substituted aryl are phenyl, substituted phenyl, naphthyl and substituted naphthyl.

The term “heteroaryl”, as used herein, refers to aromatic carbocyclic groups containing one or more heteroatoms (O, S, or N) within a ring. A heteroaryl group can be monocyclic or polycyclic. A heteroaryl group may additionally be substituted or unsubstituted. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached).

A suitable substituent on a heteroaryl group is one that does not substantially interfere with the pharmaceutical activity of the disclosed compound. A heteroaryl may have one or more substituents, which can be identical or different. Examples of suitable substituents include, but are not limited to, optionally substituted alkyl, optionally substituted alkenyl, optionally substituted alkynyl, optionally substituted cycloalkyl and cycloalkenyl, optionally substituted heterocycloalkyl and heterocycloalkenyl, halo, alkoxy, nitro, amino, carboxy, aminosulfonyl, aminocarbonyl, sulfinyl, sulfanyl, sulfonyl, hydroxy, alkoxycarbonyl, carbamate, trihalomethyl, cyano, mercapto, optionally substituted aryl and optionally substituted heteroaryl, oxo, thioxo, —NH-alkyl, —NH-alkenyl, —NH—C₃-C₁₂-cycloalkyl, —NH-aryl, —NH-heteroaryl, —NH-heterocyclic, -dialkylamino, —O—C₁-C₁₂-alkyl, —O—C₂-C₈-alkenyl, —O-cycloalkyl, —O-aryl, —O-heteroaryl, —O-heterocyciclic, —C(O)-alkyl, —C(O)-alkenyl, —C(O)-alkynyl, —C(O)-cycloalkyl, —C(O)-aryl, —C(O)-heteroaryl, —C(O)-heterocycloalkyl, —CONH₂, —CONH—-alkyl, —CONH—-alkenyl, —CONH-alkynyl, —CONH-cycloalkyl, —CONH-aryl, —CONH-heteroaryl, —CONH-heterocyclic, —O-alkyl, —OCO₂-alkenyl, —OCO₂-alkynyl, —OCO₂-cycloalkyl, —OCO₂-aryl, —OCO₂-heteroaryl, —OCO₂-heterocycloalkyl, —CO₂-alkyl, —CO₂-alkenyl, —CO₂-alkynyl, CO₂-cycloalkyl, —CO₂-aryl, CO₂-heteroaryl, CO₂-heterocyloalkyl, —OCONH₂, —OCONH-alkyl, —OCONH-alkenyl, —OCONH-alkynyl, —OCONH-cycloalkyl, —OCONH-aryl, —OCONH-heteroaryl, —OCONH-heterocyclic, —NHC(O)H, —NHC(O)-alkyl, —NHC(O)-alkenyl, —NHC(O)-alkynyl, —NHC(O)-cycloalkyl, —NHC(O)-aryl, —NHC(O)-heteroaryl, —NHC(O)-heterocyclic, —NHCO₂—-alkyl, —NHCO₂-alkenyl, —NHCO₂—-alkynyl, —NHCO₂-cycloalkyl, —NHCO₂-aryl, —NHCO₂-heteroaryl, —NHCO₂-heterocyclic, —NHC(O)NH₂, —NHC(O)NH-alkyl, —NHC(O)NH-alkenyl, —NHC(O)NH-alkynyl, —NHC(O)NH-cycloalkyl, —NHC(O)NH-aryl, —NHC(O)NH-heteroaryl, —NHC(O)NH-heterocyclic, —S(O)-alkyl, —S(O)—-alkenyl, —S(O)-alkynyl, —S(O)-cycloalkyl, —S(O)-aryl, —S(O)-heteroaryl, —S(O)-heterocyclic, —SO₂NH₂, —SO₂NH-alkyl, —SO₂NH-alkenyl, —SO₂NH—-alkynyl, —SO₂NH—-cycloalkyl, —SO₂NH-aryl, —SO₂NH-heteroaryl, —SO₂NH-heterocyclic, —NHSO₂—-alkyl, —NHSO₂-alkenyl, —NHSO₂-alkynyl, —NHSO₂-cycloalkyl, —NHSO₂-aryl, —NHSO₂-heteroaryl, —NHSO₂-heterocycloalkyl, —CH₂NH₂, —CH₂SO₂CH₃, -arylalkyl, -heteroarylalkyl, -heterocycloalkyl, polyalkoxyalkyl, —SH, —S-alkyl, —S-alkenyl, —S-alkynyl, —S-cycloalkyl, —S-aryl, —S-heteroaryl, —S-heterocycloalkyl, and the like. In a particular aspect, of the invention, the compound has the structure of Formula (I).

In one aspect, the compound has the Formula (I) wherein n is 1. In a further embodiment, the compound has the Formula (I) wherein n is 1 and R₁ and R₂ are each independently selected from the group consisting of hydrogen, C1-C4 alkyl and OR₉ is O—C1-C4 alkyl. In another embodiment, the compound has the Formula (I) wherein n is 1 and R₁ and R₂ are each independently selected from the group consisting of hydrogen, C1-C4 alkyl and OR₉, wherein OR₉ is O—C1-C4 alkyl, and R₄ is heteroaryl optionally substituted with one or more R₈. In a further embodiment, the heteroaryl is pyridinyl wherein said pyridinyl is optionally substituted with one or more R₈. In a further embodiment, R₈ is selected from the group consisting of C1-C4 alkyl and OR₉, wherein R₉ is C1-C4 alkyl.

In additional embodiments, the compound having a structure of Formula (I), is selected from the group consisting of omeprazole, esomeprazole, lansoprazole, rabeprazole, pantoprazole, leminoprazole, single enantiomers thereof, pharmaceutically acceptable salts thereof and mixtures thereof. Omeprazole, esomeprazole, lansoprazole, rabeprazole and pantoprazole have been marketed under the trade names PRILOSEC®, NEXIUM®, PREVACID®, ACIPHET®, and PROTONIX®, respectively.

Other exemplary compounds of Formula (I) include those having the structure of Formula (Ia) shown below, wherein R₁-R₅ are as shown below in Table 1.

Compounds that showed chemorepulsion that was at least 3-fold greater than media (according to the methods shown in Example 1) are shown with a “*” in Tables 1-4. As used herein, the abbreviations “Me,” “Et” and “Ph” mean methyl, ethyl and phenyl, respectively.

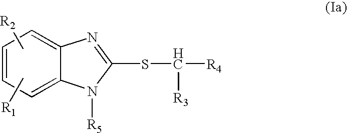

TABLE 1

| | R₁ | R₂ | R₃ | R₅ | R₄ |
|---|---|---|---|---|---|
| 60* | H | OEt | H | H | 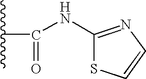 |
| 79* | H | OCH₃ | H | H | 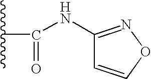 |
| 80* | H | OCH₃ | H | H | 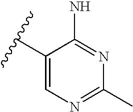 |
| 81* | H | OCH₃ | H | H | 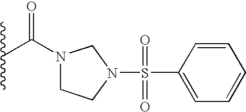 |
| 11* | H | OCH₃ | H | H | 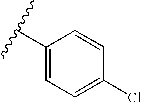 |
| 12* | H | NO₂ | H | H | 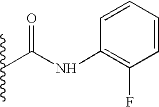 |
| 15* | H | OCH₃ | H | H | 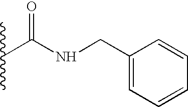 |
| 16* | H | OCH₃ | H | H | 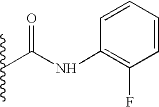 |
| 17* | H | OCH₃ | H | H | 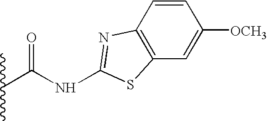 |
| 20* | H | OCH₃ | H | H | 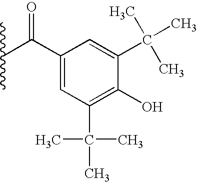 |
| 21* | H | OCH₃ | H | H | 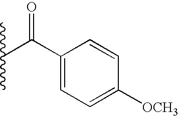 |
| 22* | H | OCH₃ | H | H | 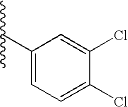 |
| 23* | H | OCH₃ | H | H | 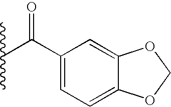 |
| 102* | H | OCH₃ | H | H | 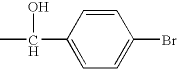 |
| 24* | H | OCH₃ | H | H | 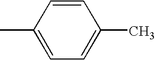 |
| 33* | H | NO₂ | H | H | 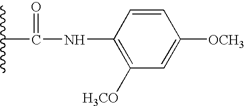 |
| 36* | H | OCH₃ | H | H | 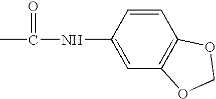 |
| 37* | H | OCH₃ | H | H | 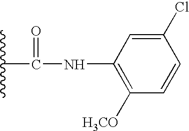 |
| 38* | H | OCH₃ | H | H | 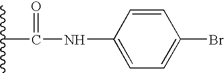 |
| 39* | H | OCH₃ | H | H | 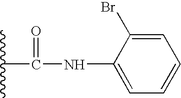 |
| 40* | H | OCH₃ | H | H | 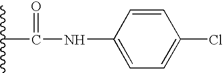 |
| 41* | H | OCH₃ | H | H | 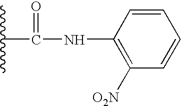 |
| 42* | H | OCH₃ | H | H | 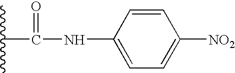 |
| 45* | H | OCH₃ | H | H | 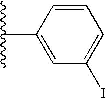 |
| 56* | H | OEt | H | H | 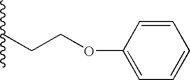 |
| 58* | H | OEt | H | H | 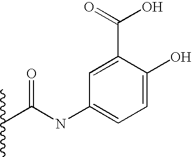 |
| 59* | H | NO₂ | H | H | 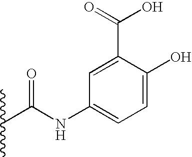 |
| 62* | H | OEt | H | H | 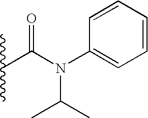 |
| 63* | H | OEt | H | H | 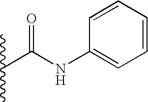 |
| 109* | OCH₃ | H | H | H | 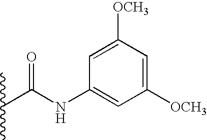 |
| 110* | OCH₃ | H | H | H | 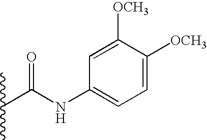 |
| 111* | OCH₃ | H | H | H | 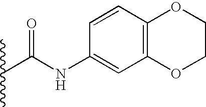 |
| 112* | OCH₃ | H | H | H | 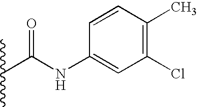 |
| 69* | OCH₃ | H | H | H | 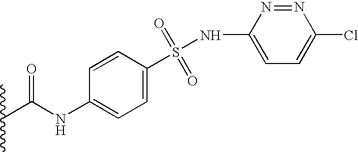 |
| 70* | OCH₃ | H | H | H | 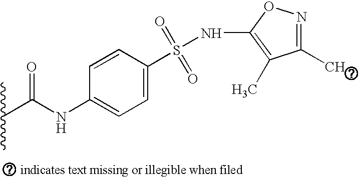 |
| 71* | H | OEt | H | H | 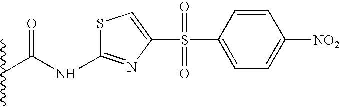 |
| 84* | H | OCH₃ | H | H | 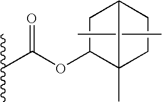 |
| 89* | H | OCH₃ | H | H | 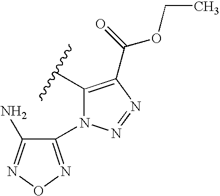 |
| 91* | H | OEt | H | H | 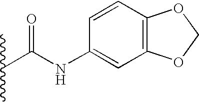 |
| 92* | H | OEt | H | H | 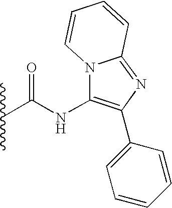 |
| 93* | H | OEt | H | H | 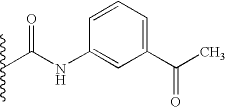 |
| 96* | H | NO₂ | H | H | 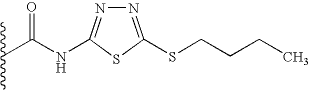 |
| 97* | H | NO₂ | H | H | 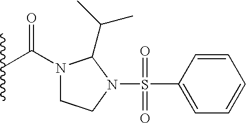 |
| 98* | H | OEt | H | H | 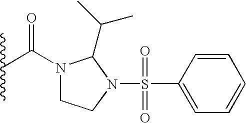 |
| 100* | H | OCH₃ | H | H | 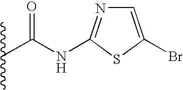 |
| 101* | H | OEt | Ph | H | 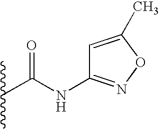 |
| 107* | OCH₃ | H | H | H | 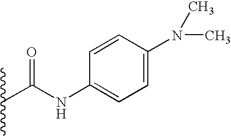 |
| 133* | H | OCH₃ | H | H | 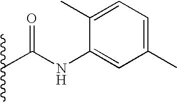 |
| 134* | H | OCH₃ | H | H | 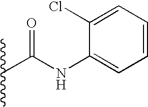 |
| 137* | H | N—(Me)₂ | H | H | 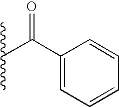 |
| 138* | H | OEt | H | H | 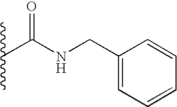 |
| 139* | H | OCH₃ | H | H | 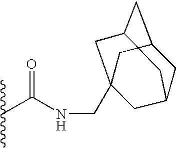 |
| 159* | H | OEt | H | H | 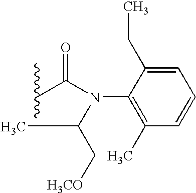 |
| 160* | H | OEt | H | H | 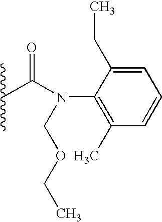 |
| 154* | H | OCH₃ | H | H | 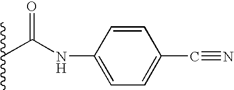 |
| 142* | OCH₃ | H | H | H | 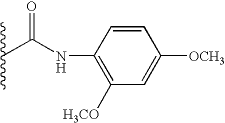 |
| 143* | OCH₃ | H | H | H | 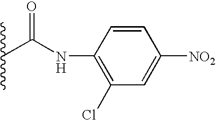 |
| 144* | OCH₃ | H | H | H | 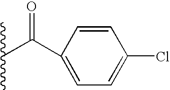 |
| 4 | H | OCH₃ | H | H | 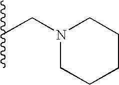 |
| 5 | H | OCH₃ | H | H | 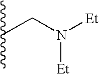 |
| 6 | H | OCH₃ | H | H | 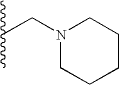 |
| 7 | H | OCH₃ | H | H | 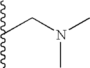 |
| 9 | H | OCH₃ | H | CH₃ | 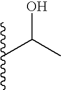 |
| 10 | H | OCH₃ | H | CH₃ | 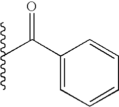 |
| 18 | H | OCH₃ | H | H | 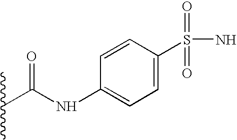 |
| 19 | H | OCH₃ | H | H | 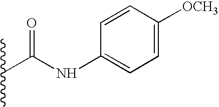 |
| 26 | H | OCH₃ | H | CH₃ | 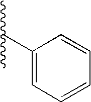 |
| 27 | H | OCH₃ | H | H | 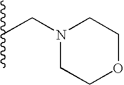 |
| 30 | H | NO₂ | H | H | 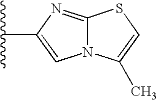 |
| 32 | H | OCH₃ | H | H | 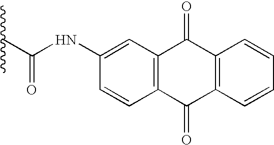 |
| 35 | H | NO₂ | H | H | 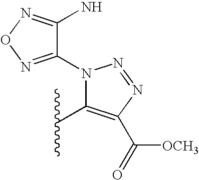 |
| 46 | H | OCH₃ | H | H | 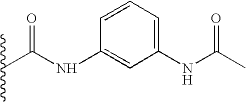 |
| 48 | H | OEt | H | H | 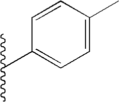 |
| 49 | H | OCH₃ | H | H | 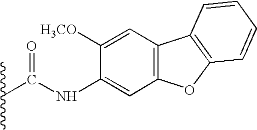 |
| 50 | H | NO₂ | H | H | 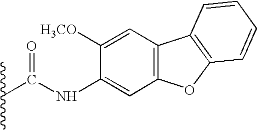 |
| 52 | H | OCH₃ | H | H | 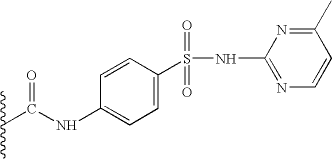 |
| 53 | H | OCH₃ | H | H | 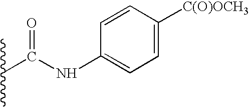 |
| 55 | H | OEt | H | H | 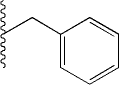 |
| 57 | H | OEt | H | H | 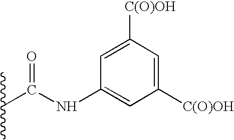 |
| 61 | OEt | H | H | H | 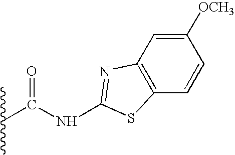 |
| 64 | H | OCH₃ | H | H | 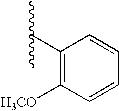 |
| 73 | OEt | H | H | H | 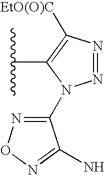 |
| 74 | H | OEt | H | H | 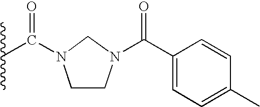 |
| 75 | H | NO₂ | H | H | 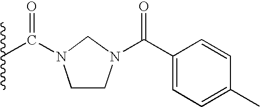 |
| 76 | H | OCH₃ | H | H | 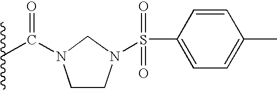 |
| 77 | H | OEt | H | H | 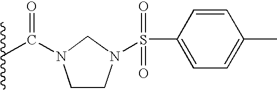 |
| 82 | H | OEt | H | H | 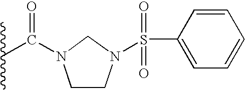 |
| 85 | H | OCH₃ | H | H | 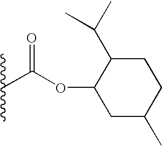 |
| 90 | H | OCH₃ | H | H | 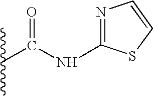 |
| 94 | H | NO₂ | H | H | 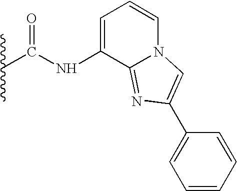 |
| 95 | H | OEt | H | H | 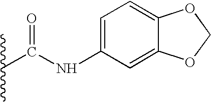 |
| 106 | OCH3 | H | H | H | 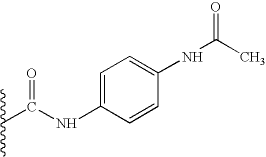 |
| 108 | OCH₃ | H | H | H | 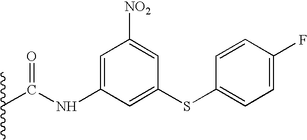 |
| 113 | OCH₃ | H | H | 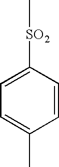 | 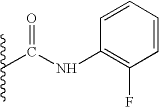 |
| 114 | H | OCH₃ | H | 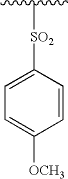 | 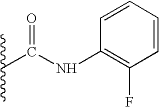 |
| 115 | H | OCH₃ | H | 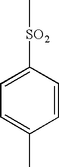 | 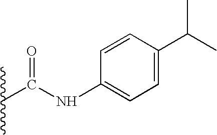 |
| 116 | H | OCH₃ | H | 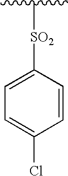 | 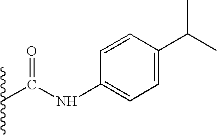 |
| 117 | H | OCH₃ | H | 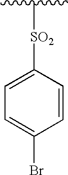 | 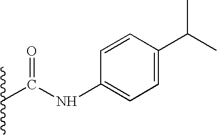 |
| 118 | H | OCH₃ | H | 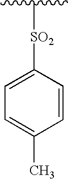 | 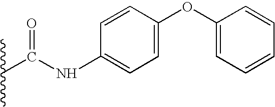 |
| 119 | H | OCH₃ | H | 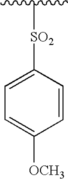 | 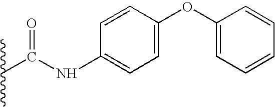 |
| 120 | H | OCH₃ | H | 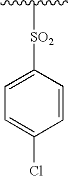 | 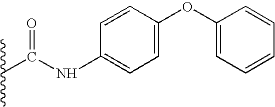 |
| 121 | H | OCH₃ | H | 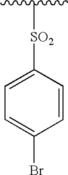 | 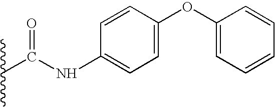 |
| 122 | H | OCH₃ | H | 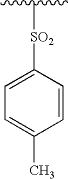 | 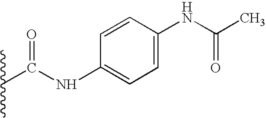 |
| 123 | H | OCH₃ | H | 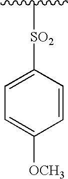 | 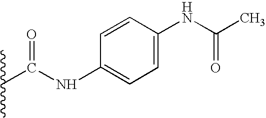 |
| 124 | H | OCH₃ | H | 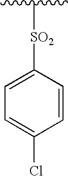 | 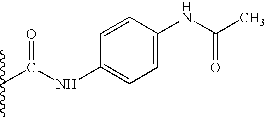 |
| 125 | H | OCH₃ | H | 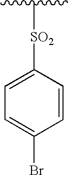 | 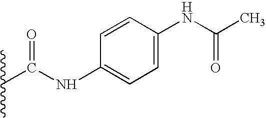 |
| 140 | H | OCH₃ | H | H | 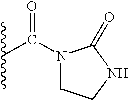 |
| 141 | H | NO₂ | H | H | 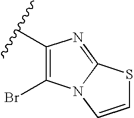 |
| 148 | H | NO₂ | H | H | 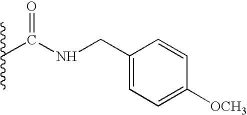 |
| 149 | OCH₃ | H | H | H | C(O)OH |
| 153 | H | NO₂ | H | H | C(O)OH |
| 155 | H | OCH₃ | H | H | 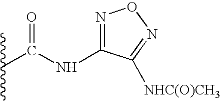 |
| 156 | H | OCH₃ | H | H | 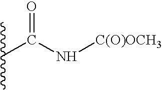 |
| 158 | H | OEt | H | H | 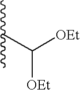 |
| 87 | H | O(CH₂)₁₁CH₃ | H | H | 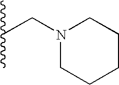 |
| 104 | H | NO₂ | H | H | 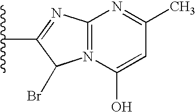 |
| 78 | H | NO₂ | H | H | 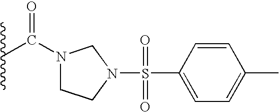 |
| 13 | H | NO₂ | H | H | 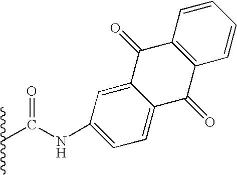 |
| 14 | H | NO₂ | H | H | 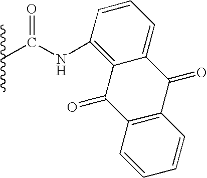 |
| 31 | H | NO₂ | H | H | 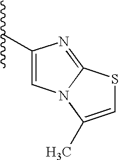 |
| 8 | H | OCH₃ | H | H | C(O)C(CH₃)₃ |

In certain aspects, the compound has the Formula (Ia). In an additional embodiment, the compound has the Formula (Ia) wherein R₁ and R₂ are each independently selected from the group consisting of hydrogen, OR₉, and NO₂. In another embodiment, the compound has the Formula (Ia) wherein R₃ is hydrogen. In an additional embodiment, the compound has the Formula (Ia) wherein R₄ is selected from the group consisting of C1-C10 alkyl, C1-C10 alkyl substituted with one or more R₇, C(O)R₆, C(O)OR₆, C(O)NR₆R_{6′}, aryl and heteroaryl, wherein said aryl and heteroaryl are each optionally substituted by one or more R₈. In a further embodiment, R₄ is C(O)NR₆R_{6′} and R₆ and R_{6′} are each independently selected from the group consisting of hydrogen, aryl and heteroaryl, wherein said aryl and heteroaryl are each optionally substituted by one or more R₈, or R₆ and R_{6′} are taken together to form a 3-6 membered ring wherein the ring is optionally substituted with one or more R₈. In yet another embodiment, R₄ is C(O)R₆ wherein R₆ is aryl or heteroaryl wherein said aryl and heteroaryl are each optionally substituted with one or more R₈. In a further embodiment, the compound has the Formula (Ia) and is selected from the group consisting of Compounds 60, 79, 80, 81, 110, 17, 20, 21, 22, 23, 102, 24, 112, 111, 11, 12, 15, 16, 33, 36, 37, 38, 39, 40, 41, 42, 45, 56, 58, 59, 62, 63, 109, 69, 70, 71, 84, 89, 89, 91, 92, 93, 96, 97, 98, 100, 101, 107, 133, 134, 138, 139, 159, 160, 154, 142, 143 and 144 (shown in Table 1).

In an additional embodiment, the compound used according to the inventive method has the Formula (II). Exemplary compounds having the Formula (II) are those with Formula (IIa), wherein R₁₀, R₁₁, R₅ and R₁₂ are as shown below in Table 2:

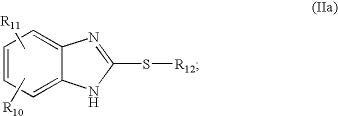

TABLE 2

| | R₁₀ | R₁₁ | R₁₂ |
|---|---|---|---|
| 54* | H | OCH₃ | 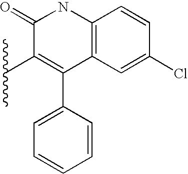 |
| 88* | H | OCH₃ | 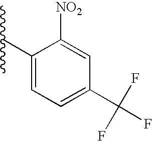 |
| 99* | H | OCH₃ | 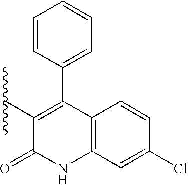 |
| 136* | 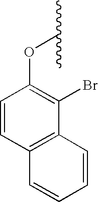 | Cl | CH₃ |
| 135* | OCH3 | H | 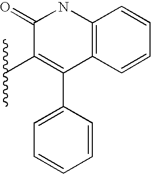 |
| 34 | H | OEt | H |
| 72 | OCH₃ | H | 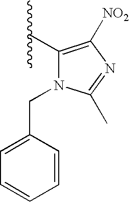 |
| 126 | H | OCH₃ | 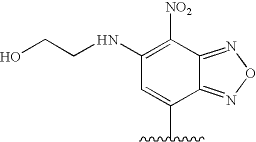 |
| 127 | H | OCH₃ | 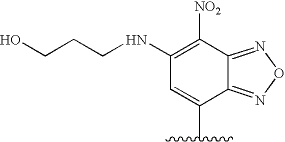 |
| 147 | 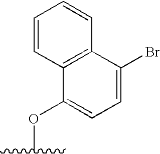 | Cl | CH₃ |

In a further embodiment, the compound has the Formula (IIa), wherein R₁₂ is selected from the group consisting of C1-C4 alkyl, C1-C4 alkyl substituted with one or more R₇, aryl and heteroaryl, wherein said aryl and heteroaryl are each optionally substituted with one or more R₈. In a further embodiment, R₁₀ and R₁₁ are each independently selected from the group consisting of hydrogen, OR₉ and NO₂. In one embodiment, R₁₀ and R₁₁ are each independently selected from the group consisting of hydrogen and OR₉, wherein OR₉ is represented by one of the following formulae:

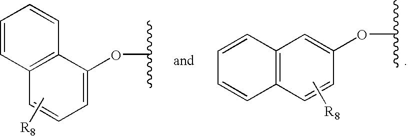

In a further embodiment, the compound has the Formula (II) and is selected from the group consisting of 54, 88, 99, 135 and 136 (shown in Table 2 above).

In yet another embodiment, the compound has the Formula (III). Exemplary compounds having the Formula (III) are those having the Formula (Ma) wherein R₁, R₂, R₅ and R₁₃ are as shown below in Table 3.

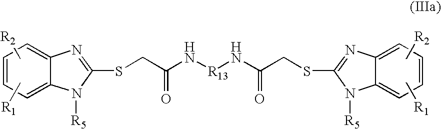

TABLE 3

| | R₁ | R₂ | R₅ | R₁₃ |
|---|---|---|---|---|
| 43* | H | OCH₃ | H | 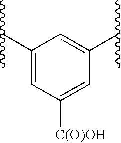 |
| 44 | H | OCH₃ | H | 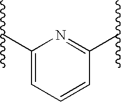 |

In one embodiment, the compound has the Formula (IIIa) wherein R₁₃ is

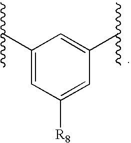

In another embodiment, the compound has the Formula (III) wherein R₁ and R₂ are each independently selected from the group consisting of hydrogen, OCH₃, OCH₂CH₃, and NO₂. In one embodiment, the compound has the structure of Compound 43 (shown in Table 3 above).

In an additional embodiment, the compound has the Formula (IV). Exemplary compounds having the Formula (IV) are those wherein R₁₀, R₁₁, and R₁₄ are as shown below in Table 4.

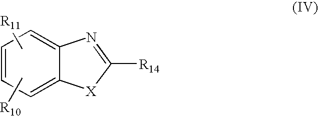

TABLE 4

| | X | R₁₀ | R₁₁ | R₁₄ |
|---|---|---|---|---|
| 128* | NH | H | OCH3 | 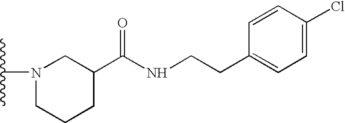 |
| 132* | S | H | NO₂ | 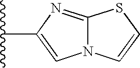 |
| 157* | NH | H | OCH₃ | 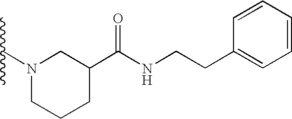 |
| 2 | NH | H | S(CH₂)₂CH₃ | NHC(O)OCH₃ |
| 25 | NH | H | OCH₃ | 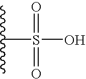 |
| 28 | NMe | H | OCH₃ | NH(CH₂)₂OH |
| 29 | NH | H | OEt | NH(CH₂)₃OH |
| 65 | NH | 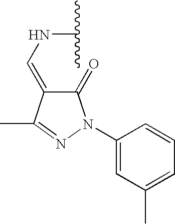 | H | H |
| 66 | NH | H | 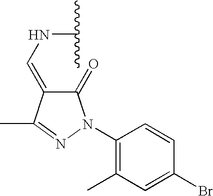 | H |
| 67 | S | H | OCH₃ | 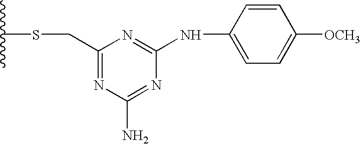 |
| 86 | NH | H | OCH₃ | 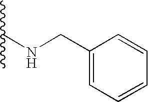 |
| 68 | S | H | OCH₃ | 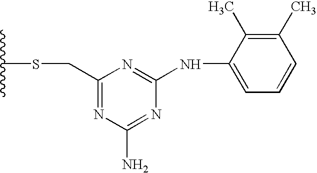 |
| 130 | NH | H | NH | 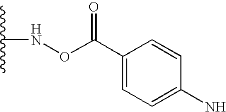 |
| 131 | NCH₃ | H | OCH₃ | NH(CH₂)₃OH |
| 145 | NH | H | S(O)₂(CH₂)₂CH₃ | NHC(O)OCH₃ |
| 151 | NH | 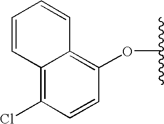 | Cl | NHC(O)OCH₃ |
| 152 | NH | Cl | 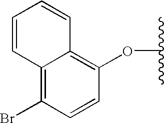 | NHC(O)OCH₃ |
| 47 | NH | 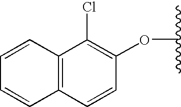 | Cl | NHC(O)OCH₃ |
| 83* | NH | H | S(CH₂)₂CH₃ | 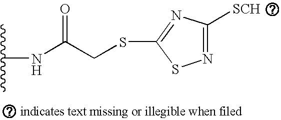 |
| 103 | O | H | NO₂ | 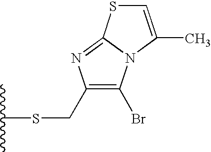 |
| 105 | O | H | NO2 | 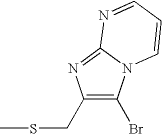 |

In a further embodiment, the compound has the Formula (IV) wherein R₁₄ is NR₆R_{6′} or heteroaryl, wherein said heteroaryl is optionally substituted with one or more R₈. In a further embodiment, R₁₄ is NR₆R_{6′} wherein R₆ and R_{6′} are taken together with the nitrogen atom to which they are attached to form a 3-6 membered ring wherein the ring is optionally substituted with one or more R₈. In yet another embodiment, the compound has the Formula (IV) wherein R₁₄ is NR₆R_{6′} or heteroaryl, wherein said heteroaryl is optionally substituted with one or more R₈ and wherein R₁₀ and R₁₁ are each independently selected from the group consisting of hydrogen, OCH₃, OCH₂CH₃ and NO₂. In another embodiment, the compound has the Formula (IV) and is selected from the group consisting of Compound 128, Compound 132, Compound 157 and Compound 83.

Pharmaceutically acceptable salts of compounds having a structure selected from the Formula (I), (II), (III) or (IV) include, but are not limited to, base addition salts, such as those described in U.S. Pat. Nos. 4,738,974, 5,690,960, 5,714,504, 5,900,424, 6,875,872. In one embodiment, the base addition salt is a lithium, sodium, potassium, magnesium or calcium salt. In a further embodiment, the base addition salt is a magnesium salt.

As used herein, “migratory cells” are those cells which are capable of movement from one place to another in response to a stimulus. Human migratory cells include those involved in the processes of cancer, immunity, angiogenesis or inflammation and also include those identified to play a role in other disease states or conditions. Migratory cells include, but are not limited to, immune cells, hematopoietic cells, neural cells, epithelial cells, mesenchymal cells, stem cells, germ cells and cells involved in angiogenesis.

Immune cells include, but are not limited to, monocytes, Natural Killer (NK) cells, dendritic cells (which could be immature or mature), subsets of dendritic cells including myeloid, plasmacytoid (also called lymphoid) or Langerhans; macrophages such as histiocytes, Kupffer's cells, alveolar macrophages or peritoneal macrophages; neutrophils, eosinphils, mast cells, basophils; B cells including plasma B cells, memory B cells, B-1 cells, B-2 cells; CD45RO (naive T), CD45RA (memory T); CD4 Helper T Cells including Th1, Th2 and Tr1/Th3; CD8 Cytotoxic T Cells, Regulatory T Cells and Gamma Delta T Cells.

Hematopoietic cells include, but are not limited to, pluripotent stem cells, multipotent progenitor cells and/or progenitor cells committed to specific hematopoietic lineages. The progenitor cells committed to specific hematopoietic lineages can be of T cell lineage, B cell lineage, dendritic cell lineage, neutrophil lineage, Langerhans cell lineage and/or lymphoid tissue-specific macrophage cell lineage. The hematopoietic cells can be derived from a tissue such as bone marrow, peripheral blood (including mobilized peripheral blood), umbilical cord blood, placental blood, fetal liver, embryonic cells (including embryonic stem cells), aortal-gonadal-mesonephros derived cells, and lymphoid soft tissue. Lymphoid soft tissue includes the thymus, spleen, liver, lymph node, skin, tonsil and Peyer's patches. In other embodiments, hematopoietic cells can be derived from in vitro cultures of any of the foregoing cells, and in particular in vitro cultures of progenitor cells.

Neural cells are cells of neural origin and include neurons and glia and/or cells of both central and peripheral nervous tissue.

Epithelial cells include cells of a tissue that covers and lines the free surfaces of the body. Such epithelial tissue includes cells of the skin and sensory organs, as well as the specialized cells lining the blood vessels, gastrointestinal tract, air passages, lungs, ducts of the kidneys and endocrine organs.

Mesenchymal cells include, but are not limited to, cells that express typical fibroblast markers such as collagen, vimentin and fibronectin.

Cells involved in angiogenesis are cells that are involved in blood vessel formation and include cells of endothelial origin and cells of mesenchymal origin.

Germ cells are cells specialized to produce haploid gametes.

In certain embodiment, the human migratory cell is an immune cell. In other embodiments, the immune cell is selected from the group consisting of lymphocytes, monocytes, neutrophils, eosinophils and mast cells. In a further embodiment, the immune cell is a neutrophil or an eosinophil.

As used herein, the terms “contact” or “contacting” means the act of touching or bringing together two entities or things in such proximity as will allow an influence of at least one on the other. The definition, while inclusive of physical contact is not so limited.

As used herein, a “chemorepellant” is an agent or stimulus that induces, elicits or triggers negative chemotaxis of a migratory cell. A “chemoattractant” is an agent or stimulus that induces, elicits or triggers positive chemotaxis (movement towards an agent or stimulus) by a migratory cell. Compounds of Formula (I), (II) and (III) have been discovered to be chemorepellants. As used herein the terms “induce,” “elicit,” and “trigger,” when referring to the activity of a chemorepellant or chemoattractant with respect to negative or positive chemotaxis, carry the same meaning. The term “agent” refers generally to any chemical compound or biologic.

Based on their ability to induce negative chemotaxis, compounds of Formula (I), (II), (III) and (IV) are useful inhibiting the induction of chemotaxis of migratory cells toward a chemotactic site. As used herein, a “chemotactic site” is a site that induces positive chemotaxis of migratory cells. Chemotactic sites include sites of inflammation, medical implants, transplants and angiogenesis.

Compounds of Formula (I), (II), (III) and (IV) are useful for inhibiting the induction of chemotaxis of migratory cells toward a site of inflammation. Inhibiting migratory cell chemotaxis toward a site of inflammation can result in a reduction or amelioration of an inflammatory response in situations such as bacterial infection, tissue injury-induced inflammation (e.g., ischemia-reperfusion injury), complement-induced inflammation, oxidative stress (e.g., hemodialysis), immune complex-induced inflammation (e.g., antibody-mediated glomerunephritis), cytokine-induced inflammation (e.g., rheumatoid arthritis), antineutrophil cytoplasmic antibodies and vasculitis (e.g., autoimmunity against neutrophil components), genetic disorders of neutrophil regulations (e.g., hereditary periodic fever syndromes), implant related inflammation, and cystic fibrosis.

In certain embodiments, the invention is a method of treating an inflammatory condition in a patient suffering therefrom comprising administering to said patient a compound of the Formula (I), (II), (III) or (IV) in a therapeutically effective amount. Inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, urticaria, acne, vasulitis, angiitis, endocarditis, arteritis, atherosclerosis, thrombophlebitis, pericarditis, myocarditis, myocardial ischemia, periarteritis nodosa, rheumatic fever, Alzheimer's disease, celiac disease, congestive heart failure, adult respiratory distress syndrome, meningitis, encephalitis, multiple sclerosis, cerebral infarction, cerebral embolism, Guillame-Barre syndrome, neuritis, neuralgia, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, Hodgkins disease and injection site reaction.

Injection site reaction is a term generally used to describe inflammation in and around a site of injection. Injection site reaction has been observed with the injection of numerous pharmaceutical agents including, but not limited, chemotherapeutic drugs, immunomodulator drugs, and vaccines. The present invention encompasses a method for the treatment or reduction of injection site reaction comprising administration of a compound of the Formula (I), (II), (III) or (IV) to the injection site. A compound of the Formula (I), (II), (III) or (IV) for example, be administered before, during or after injection. In some embodiments, the compound of the Formula (I), (II), (III) or (IV) thereof can be administered topically at the site of the injection.

In another embodiment, the invention is a method of inhibiting positive chemotaxis toward a medical implant. The medical implant can be contacted or coated with a compound having a structure selected from the Formulae (I), (II), (III) and (IV) in amount sufficient to induce negative chemotaxis of an immune cell. The compound of Formula (I), (II), (III) or (IV) can also be administered locally at the site of the medical implant. A medical implant is defined as a device or entity implanted into a surgically or naturally formed cavity of the body. Medical implants include, but are not limited to, stents, pacemakers, pacemaker leads, defibrillators, drug delivery devices, sensors, pumps, embolization coils, sutures, electrodes, cardiovascular implants, arterial stents, heart valves, orthopedic implants, dental implants, bone screws, plates, catheters, cannulas, plugs, fillers, constrictors, sheets, bone anchors, plates, rods, seeds, tubes, or portions thereof. In addition to the compound of Formula (I), (II), (III) or (IV), the medical implant can be coated with a cell-growth potentiating agent, an anti-infective agent and/or an anti-inflammatory agent.

In yet another embodiment, the invention is a method of inhibiting positive chemotaxis toward an organ transplant or tissue graft. Organ transplants and tissue grants include, but are not limited to, renal, pancreatic, hepatic, lymphoid and cardiac grafts and organs. Lymphoid grafts include a splenic graft, a lymph node derived graft, a Peyer's patch derived graft, a thymic graft and a bone marrow derived graft. In an additional embodiment, the invention is a method of treating a patient suffering from transplant or graft rejection comprising administering a compound having a structure selected from Formula (I), (II), (III) and (IV).

As discussed above, compounds of Formula (I), (II), (III) or (IV) can be used to inhibit chemotaxis toward a site of angiogenesis. A site of angiogenesis is a site where blood vessels are being formed. In one embodiment, the invention is a method of inducing negative chemotaxis of endothelial cells toward a site of angiogenesis. The invention also encompasses a method of inhibiting angiogenesis in a patient in need thereof comprising administering a compound of Formula (I), (II), (III) or (IV) in a therapeutically effective amount. In a further embodiment, the invention is a method of treating cancer or a tumor comprising administering a compound of Formula (I), (II), (III) or (IV) in an amount effective to inhibit angiogenesis. According to another aspect of the invention, a method of inhibiting endothelial cell migration to a tumor site in a subject is provided. The method involves locally administering to or contacting an area surrounding a tumor site in need of such treatment a compound of Formula (I), (II), (III) or (IV) in an amount effective to inhibit endothelial cell migration into the tumor site in the subject.

Exemplary cancers and tumors that can be treated according to the methods of the invention include, for example, biliary tract cancer; brain cancer including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma [teratomas, choriocarcinomas]), stromal tumors and germ cell tumors; thyroid cancer, including thyroid adenocarcinoma and medullar carcinoma; and renal cancer including adenocarcinoma and Wilms tumor.

The invention also encompasses a method of contraception in a patient in need thereof comprising administering a compound of Formula (I), (II), (III) or (IV) in an amount effective to inhibit migration of germ cells in the subject. According to another aspect of the invention, a method of treating infertility and premature labor is provided. The method comprises administering a compound having Formula (I), (II), (III) or (IV) in an amount effective to inhibit immune cells from migrating close to a germ cell in the subject.

The treatment methods disclosed herein involve administering, either locally or systemically, to a selected site in a subject in need of such a treatment a compound of Formula (I), (II), (III) or (IV) in an amount effective to induce negative chemotaxis of a human migratory cell. As used herein, a “therapeutically effective amount” is an amount sufficient to induce negative migration of a migratory cell and/or ameliorate a disease or condition of a patient or achieve a desired outcome. For example, a “therapeutically effective amount” in reference to the treatment of an inflammatory condition encompasses an amount sufficient to induce negative chemotaxis of an immune cell and/or ameliorate a symptom of the inflammatory condition.

In certain embodiments, the compound of Formula (I), (II), (III) or (IV) can be co-administered with a second agent (e.g., another chemoattractant or with any drug or agent which is not itself a chemoattractant). Co-administered agents, compounds, chemoattractants or therapeutics need not be administered at exactly the same time. In certain embodiments, however, the compound of Formula (I), (II), (III) or (IV) is administered substantially simultaneously as the second agent. By “substantially simultaneously,” it is meant that the compound of Formula (I), (II), (III) or (IV) is administered before, at the same time, and/or after the administration of the second agent. Second agents include, for example, anti-inflammatory agents, anti-cancer agents, anti-infective agents, immune therapeutics (immunosuppresants) and other therapeutic compounds. A second agent can be chosen based on the condition or disease to be treated. For example, in a method of treating cancer or a tumor, a compound of Formula (I), (II), (III) or (IV) can be administered with an anti-cancer agent. Similarly, in a method of treating an inflammatory condition, a compound of Formula (I), (II), (III) or (IV) can be administered with an anti-inflammatory agent, an anti-infective agent or an immunosuppressant.

An anti-infective agent is an agent which reduces the activity of or kills a microorganism and includes: Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter: Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride.

Exemplary anti-cancer agents include Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexatc; Eflorithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatini; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Podofilox; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxotere; Tecogalan Sodium; Tegafur, Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporlin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate Virlrosidine Sulfate; Vinzolidine Sulfate; Vorozole; Zeniplatin; Zinostatin; and Zorubicin Hydrochloride.

Exemplary immunosuppressants include Azathioprine; Azathioprine Sodium; Cyclosporine; Daltroban; Gusperimus Trihydrochloride; Sirolimus; and Tacrolimus.

Exemplary anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

As used herein, “treatment” and/or “treating” refer to therapeutic treatment as well as prophylactic treatment or preventative measures. The compound of Formula (I), (II), (III) and (IV) can be administered in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be preferred for treatment of a systemic disorder and oral administration may be preferred to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

Pharmaceutical compositions comprising compounds of Formula (I), (II), (III) and (IV) can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal.

In one embodiment, the pharmaceutical composition can be administered orally. For the purpose of oral therapeutic administration, the pharmaceutical compositions can be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

The pharmaceutical composition can also be administered by nasal administration. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

The invention is illustrated by the following examples which are not intended to be limiting in any way.

### EXEMPLIFICATION

### Example 1

### Method of Determining Chemorepulsive and Chemoattractive Activity

The chemorepulsive activity of esomeprazole (Example 2), omeprazole (Example 3), rabeprazole (Example 5) and compounds from a library of 154 chemical analogs of omeprazole (Example 4) were determined as follows:
1. Prior to beginning the assay, the following were prepared:
   a. 0.5% Fetal Calf Serum (FCS) in Iscove's Modified Dulbecco's Medium (IMDM) (Assay Medium) (Both from ATCC).
   b. Migratory cells at a concentration of 2×10⁷ cells/ml in Assay Medium.
   c. Four serial (10-fold) dilutions of the ligand of interest in Assay Medium. All stocks were diluted initially 1:100 into Assay Medium and subsequently made into 10 fold dilutions (100 uM, 10 uM, 1 uM, 0.1 uM). The library of 154 chemical analogs was resuspended from powder to 25 mM stocks with DMSO. The same dilutions were followed.
2. The assay plates are Neuroprobe ChemoTx plates, part number 206-3 (3 um pore size) for neutrophils.
3. The plates were removed from packaging, leaving the membrane behind.
4. 31 μl of the following solutions were pipetted into each well:
   a. For media controls and for chemorepulsion samples, Assay Medium was used.
   b. For chemoattraction samples, appropriate dilution of ligand was used.
5. A sterile needle was used to pop any and all small bubbles present in each well.
6. The membrane was carefully placed onto the plate, starting at one side and then slowly lowering the other edge onto the plate.
7. 29 μl of the following were pipetted onto the top of each circle:
   a. For media controls and chemoattraction samples, use Assay Medium.
   b. For chemorepulsion samples, use the appropriate dilution of ligand.
8. 2 μl of cells (40,000 cells) were added to each bubble of liquid from step 7.
9. A needle was used to pop all air bubbles.
10. The plate was covered with the supplied lid and incubated for the desired time at 37° C. in 5% CO₂. Unless otherwise indicated, the incubation time was 1 hour for neutrophils and 3 hours for T cells. For monocytes and B cells, the incubation time was 2 hours.
11. After the desired assay time, the liquid was removed from the top of the plate using a Kimwipe. At this point, the plate was stored (with the membrane in place) for up to 2 hours at 4° C.
12. The membrane was carefully removed from the top of the plate and discard. The plate was examined under a microscope to look for ligand crystallization, contamination and overall migration.
13. White read plates were preloaded with 25 ul PBS.
14. Using a multichannel pipettor, 5 ul of Cell Titer Glo (Promega # G7572) was added to each well.
15. Using a multichannel pipettor set at 30 ul, lysed cell solution was transferred to white read plates pre-loaded with PBS.
16. The plate was read using the BioTek Synergy4 plate reader in order to quantify the number of migrated cells.

Compounds from the chemical analog library were screened once in CR mode as described. Some of the compounds that exhibited large repulsion were also screened in CA mode. Compounds that were characterized as exhibiting large repulsion were those that subjectively showed greater than 3-fold greater chemorepulsion than media.

### Example 2

### Esomperazole Induces Negative Induction of Human Neutrophils

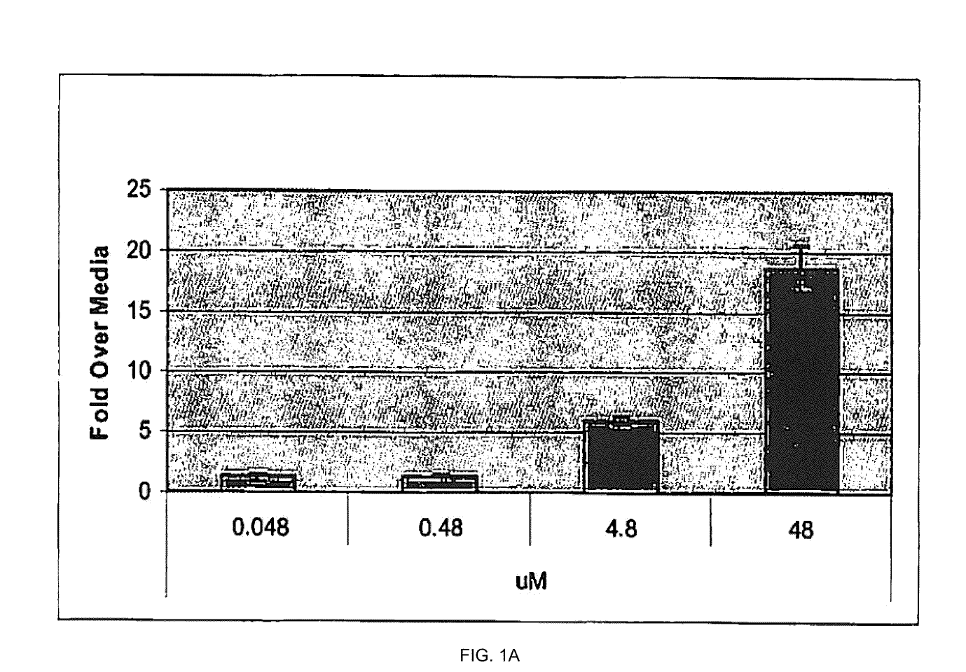

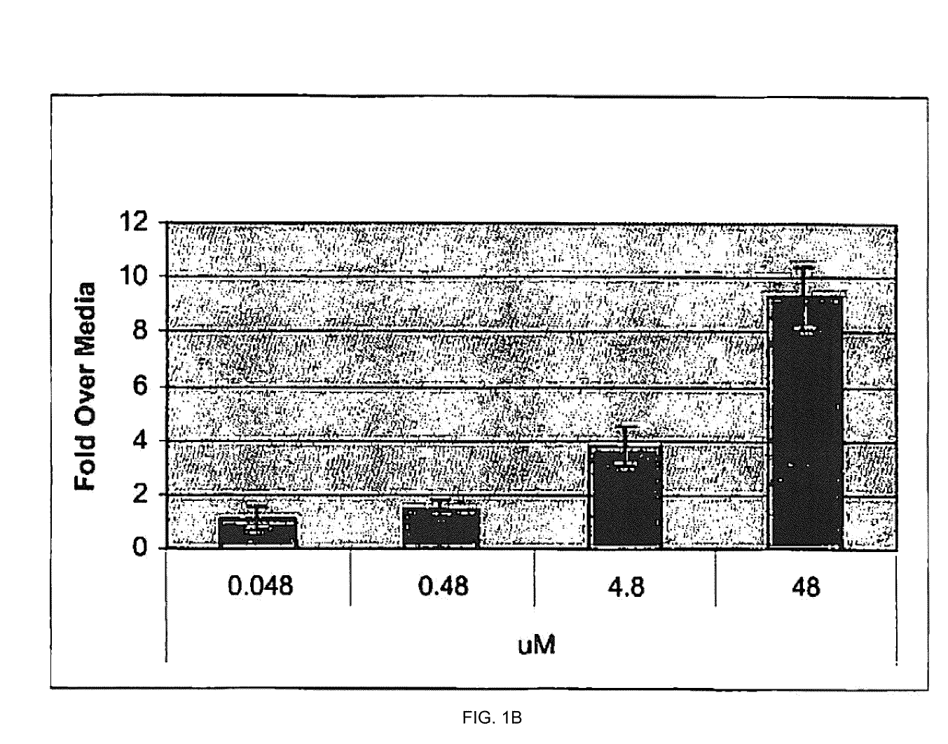

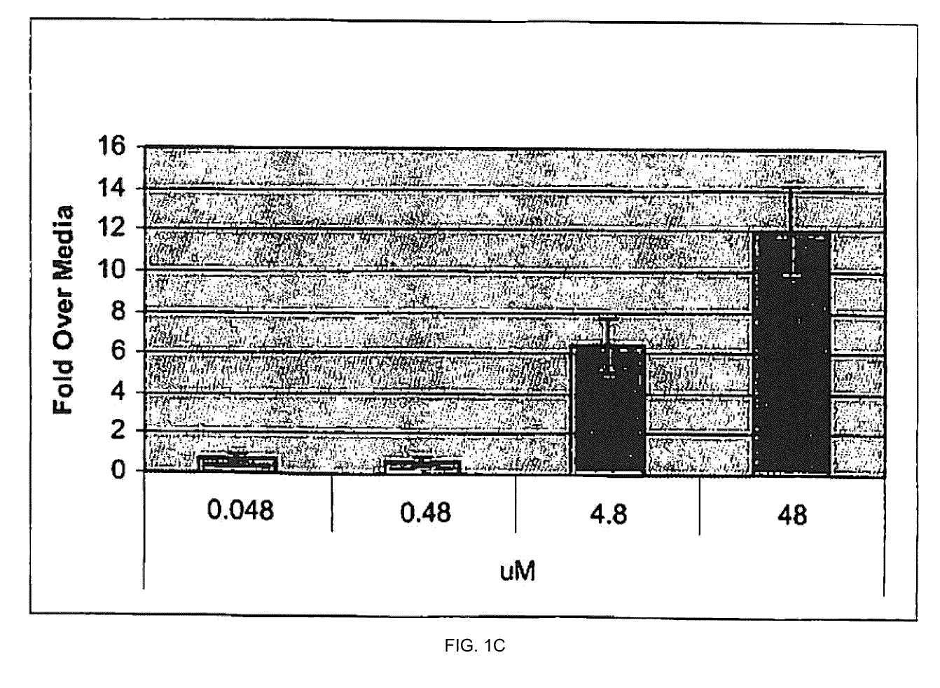

As shown in FIGS. 1A, 1B and 1C, neutrophils showed chemorepulsion in response to the administration of esomeprazole at 0.048, 0.48, 4.8 or 48 uM esomeprazole. The induction of chemotaxis was dose-dependent.

### Example 3

### Omeprazole Induces Negative Induction of Human Neutrophils

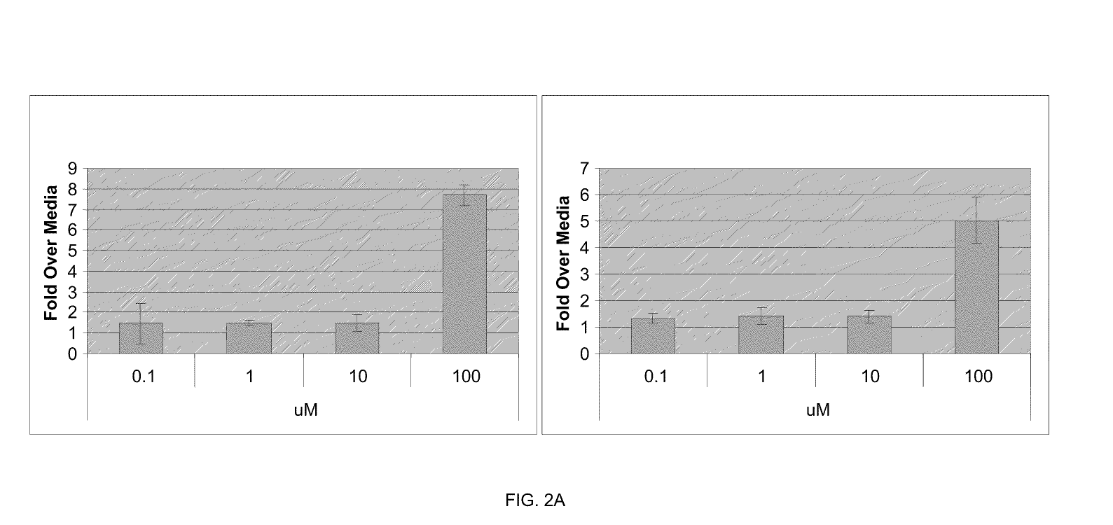

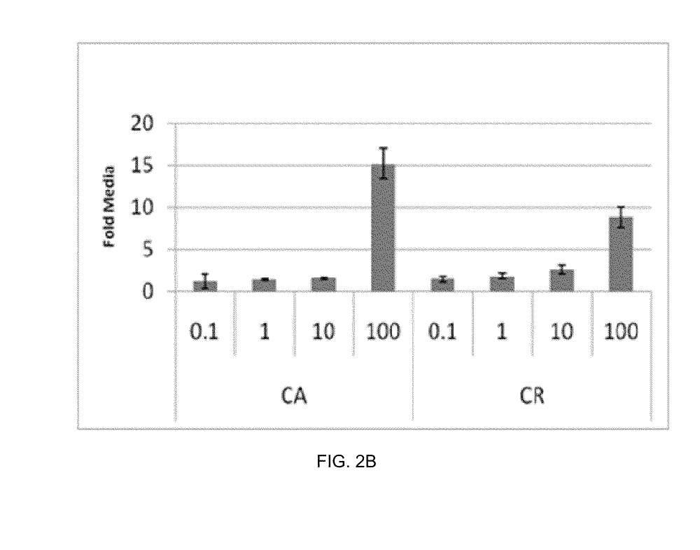

As shown in FIG. 2, neutrophils treated with omeprazole showed greater than 5-fold chemorepulsion than cells treated with media alone. This chemorepulsion was dose-dependent.

### Example 4

### Compounds from Omeprazole Derivative Library Induce Negative Induction of Human Neutrophils

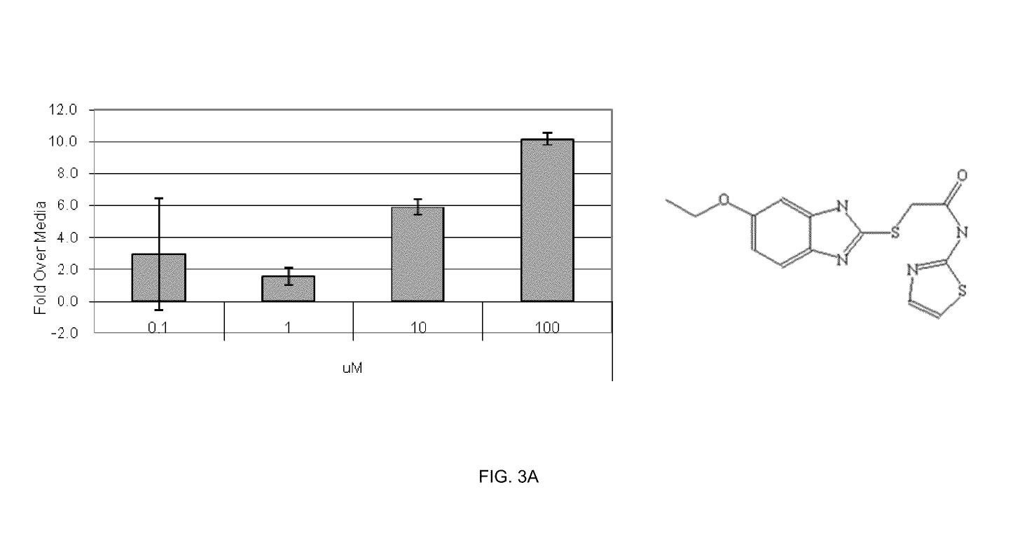

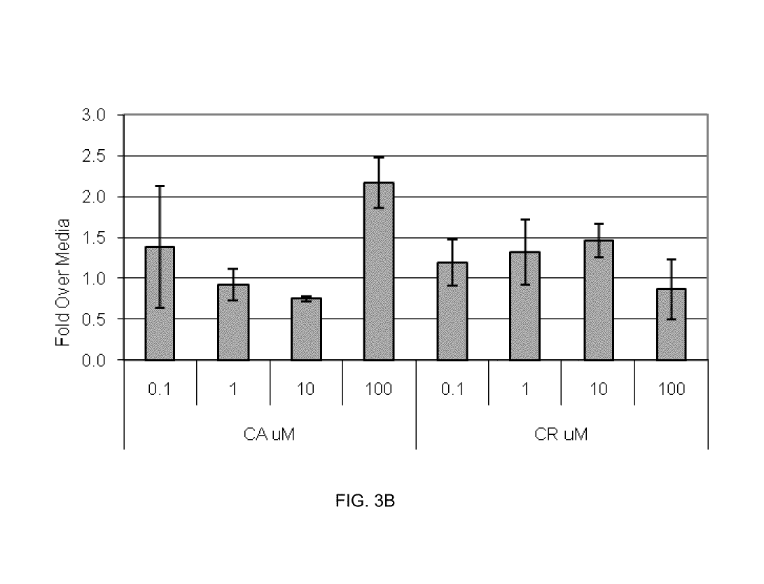

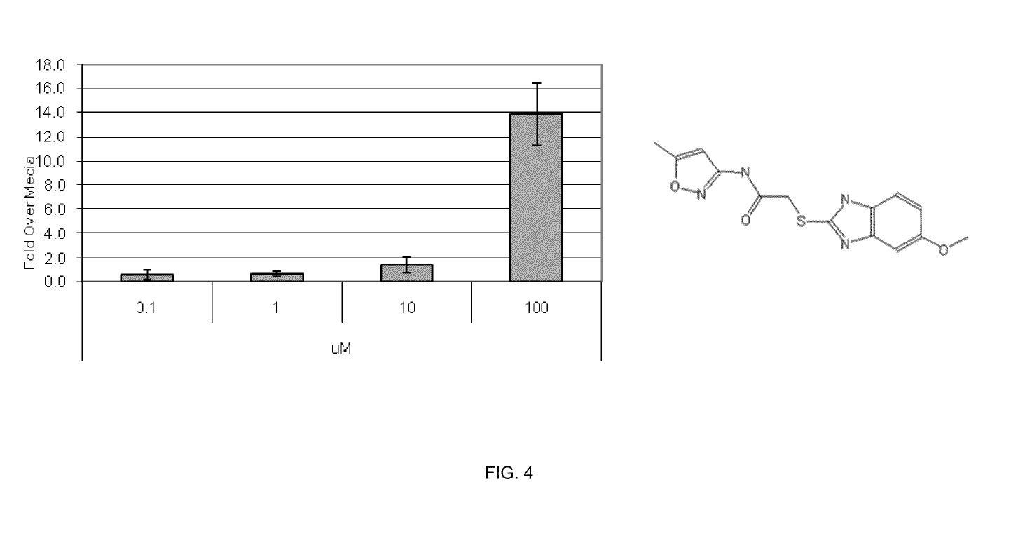

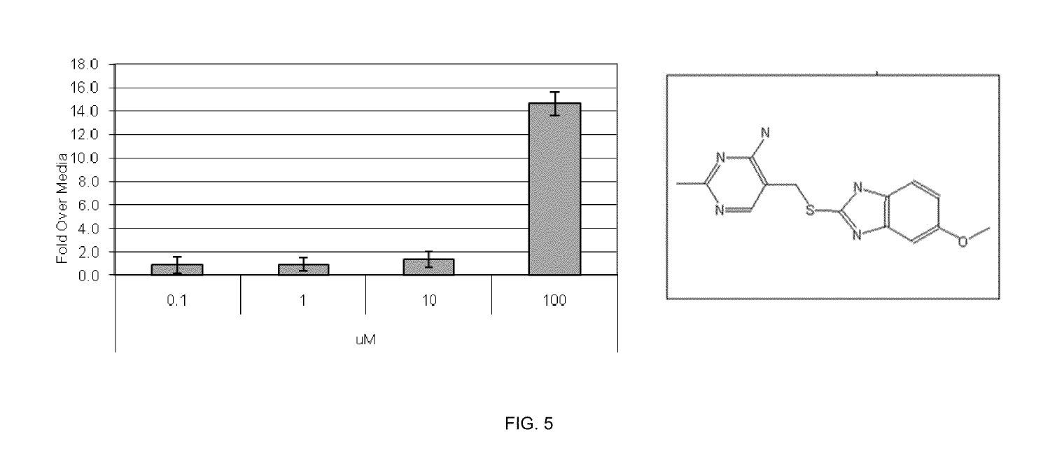

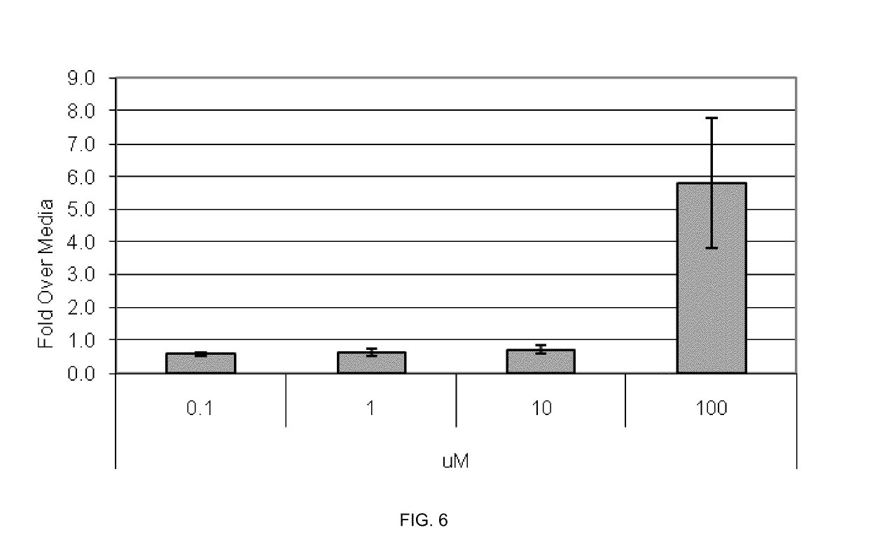

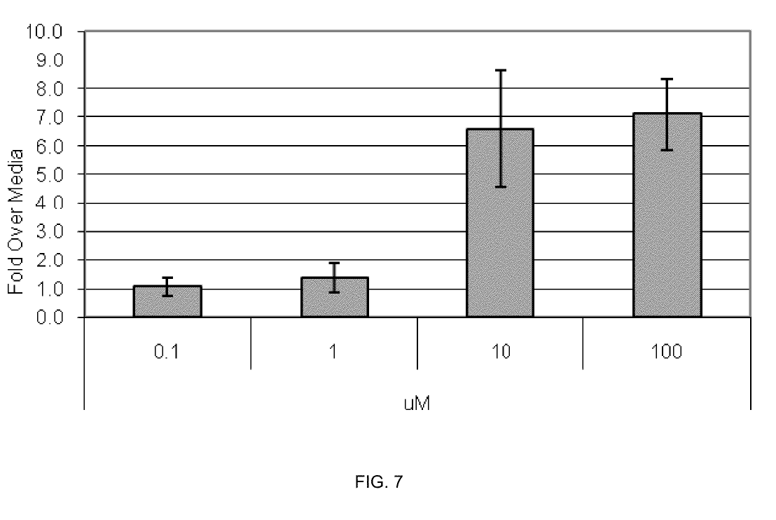

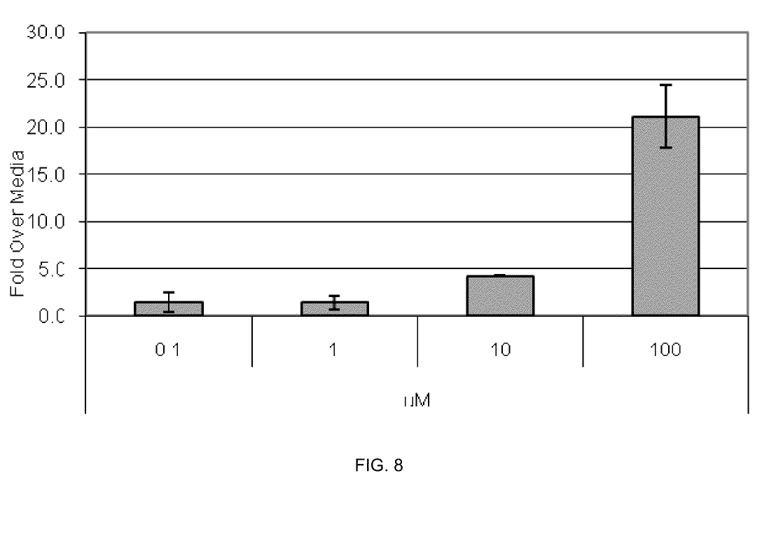

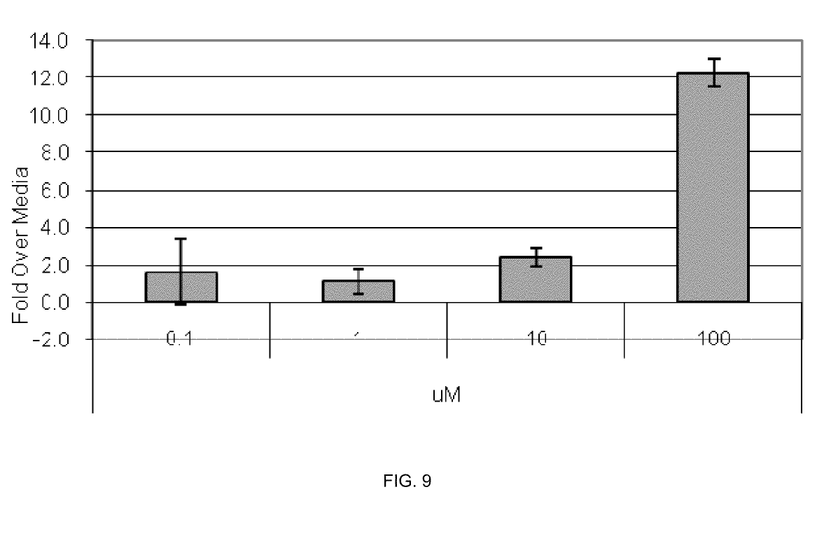

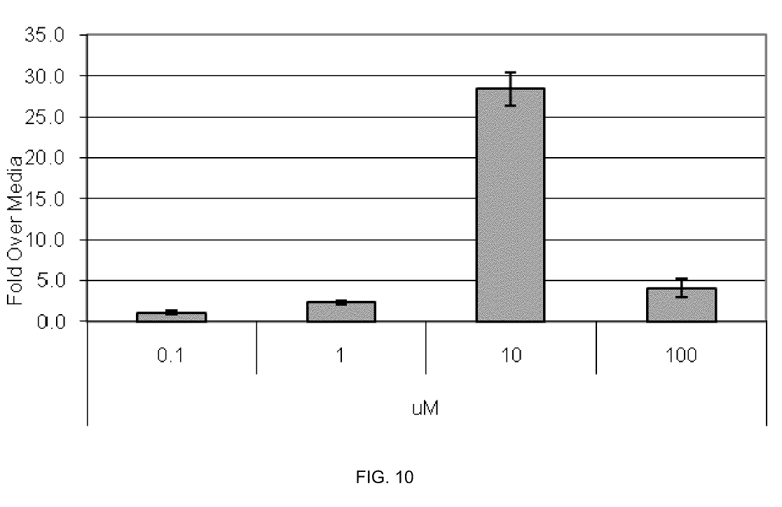

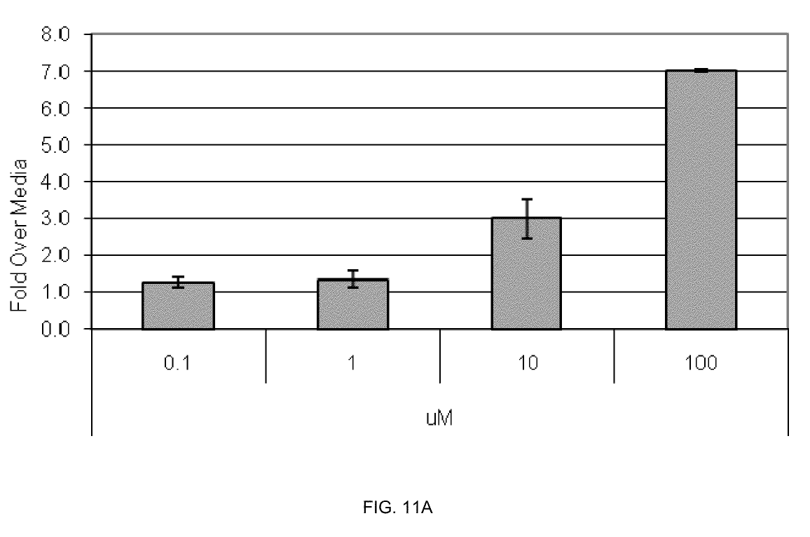

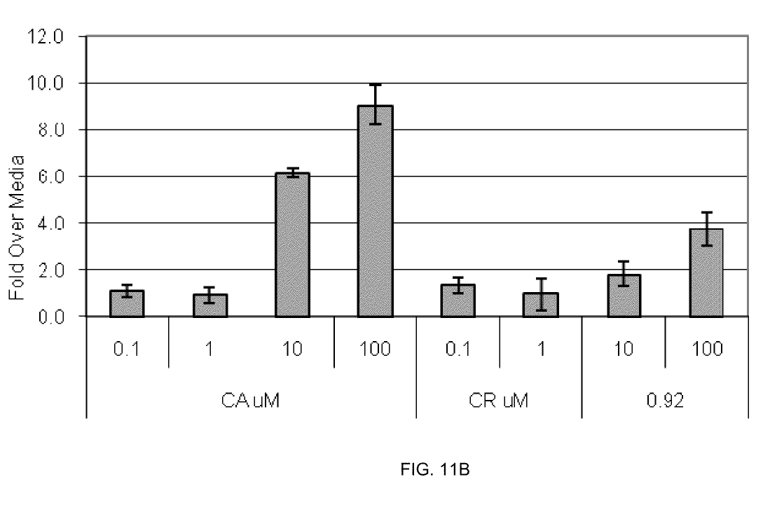

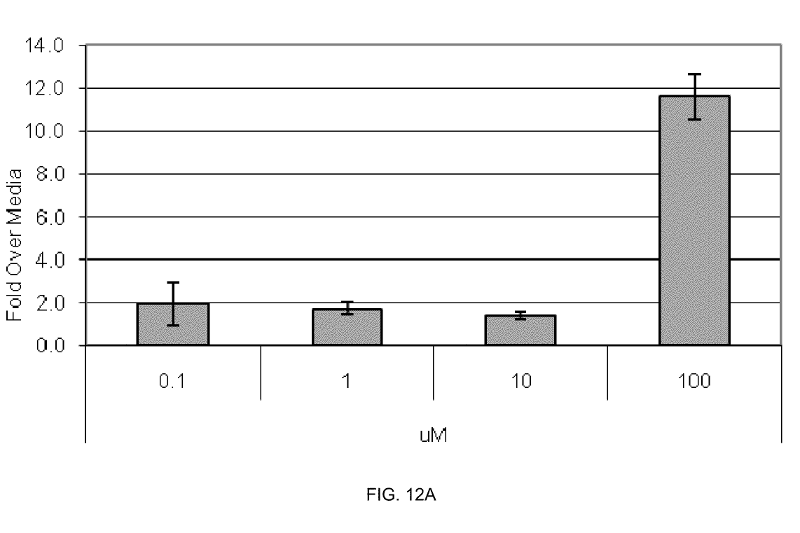

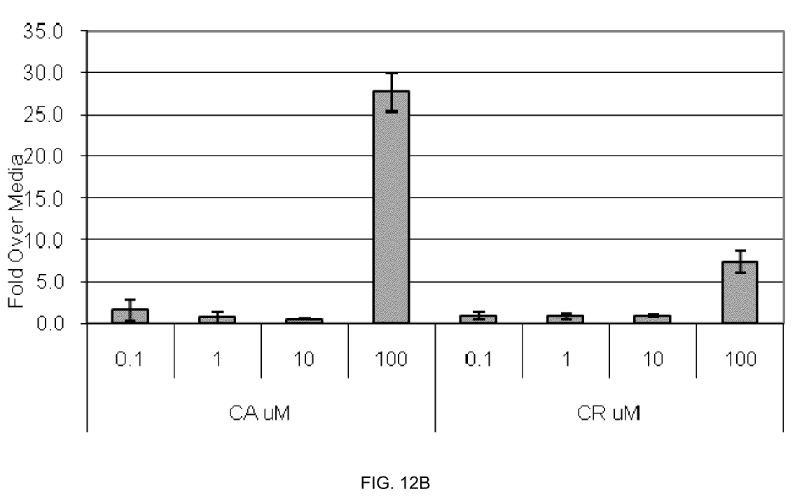

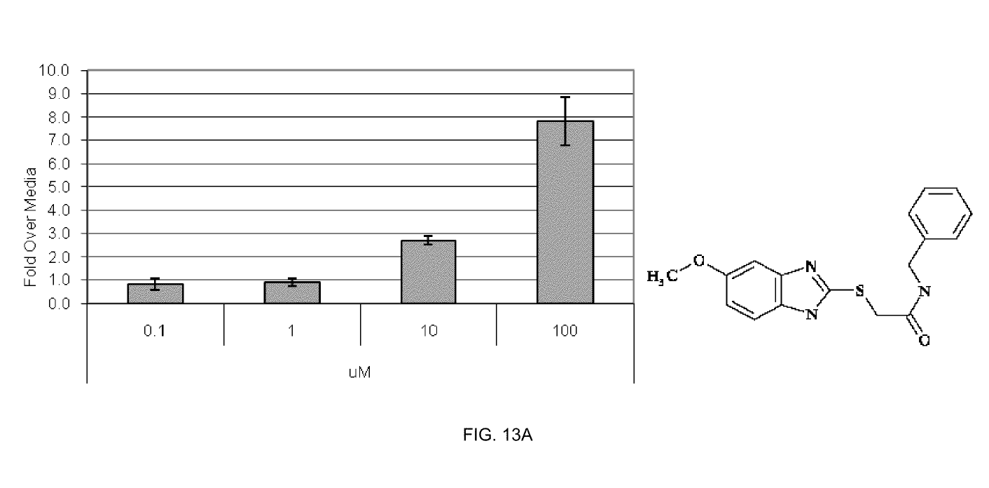

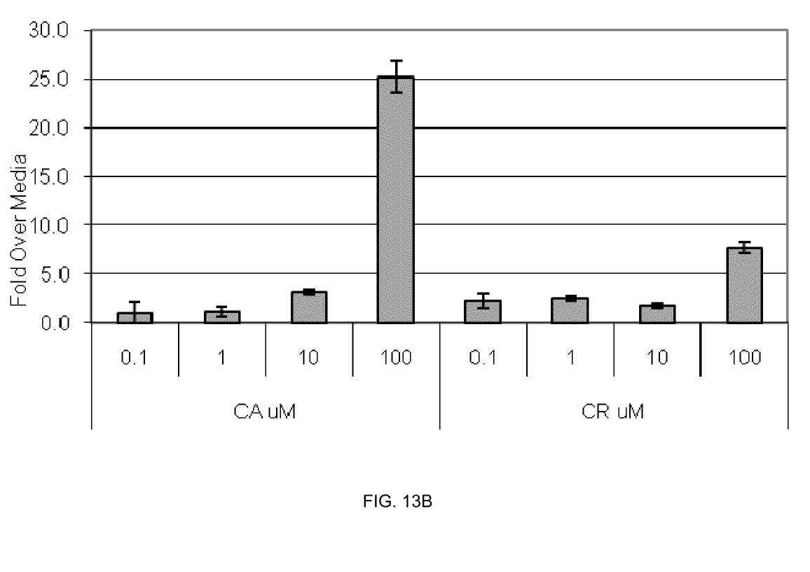

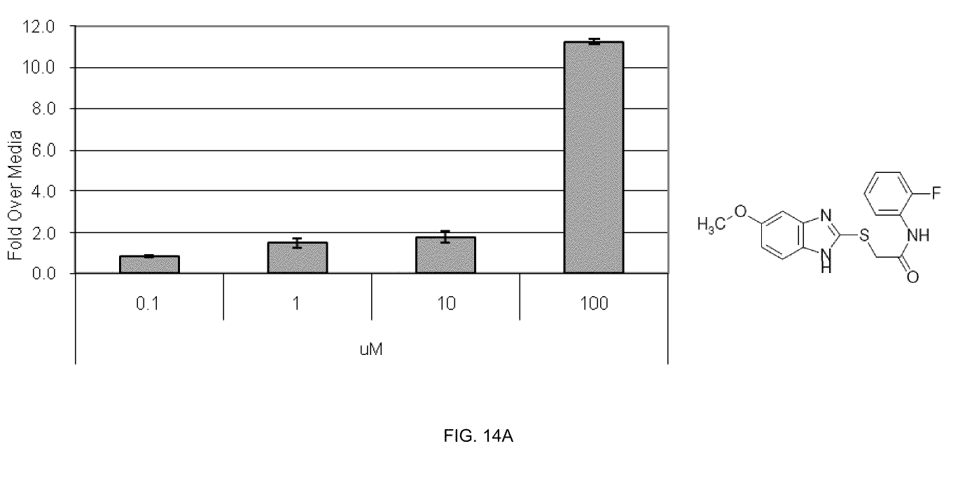

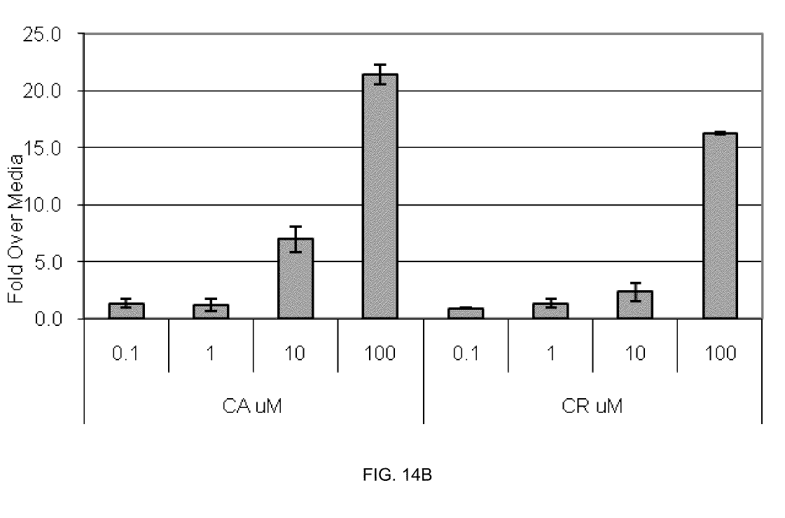

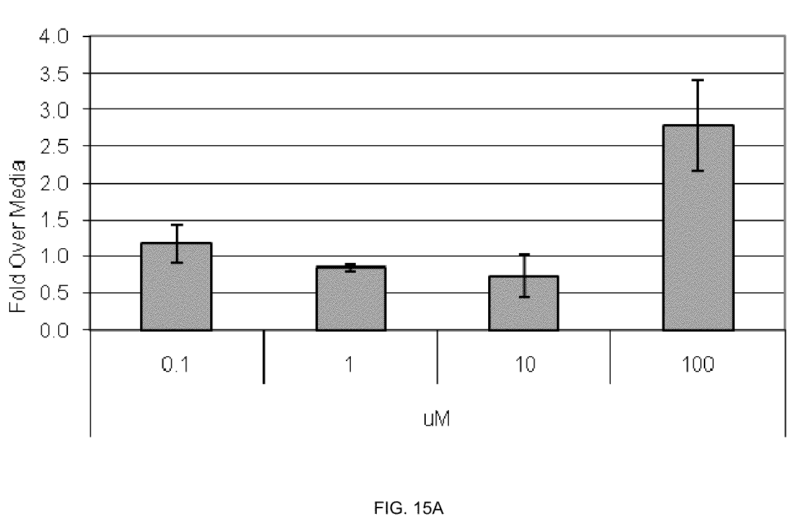

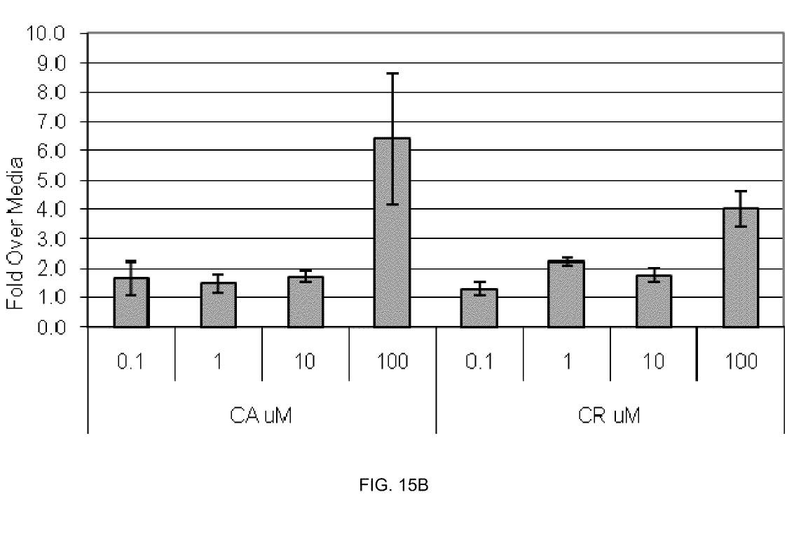

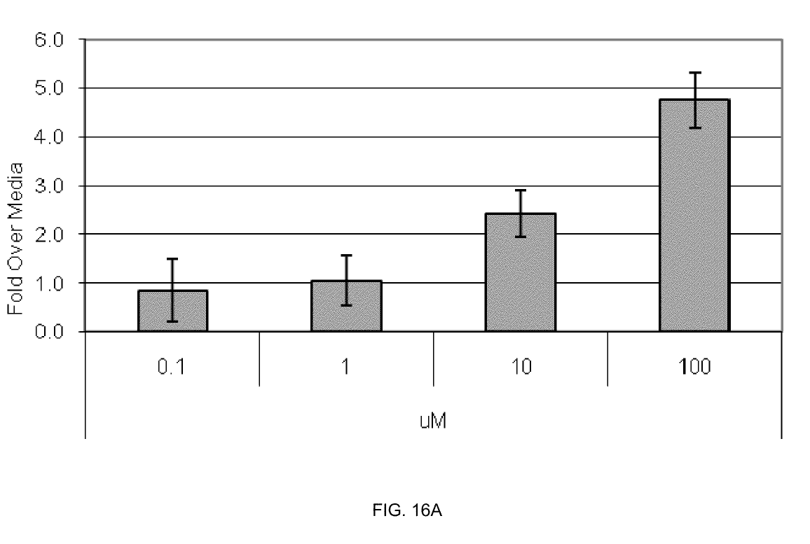

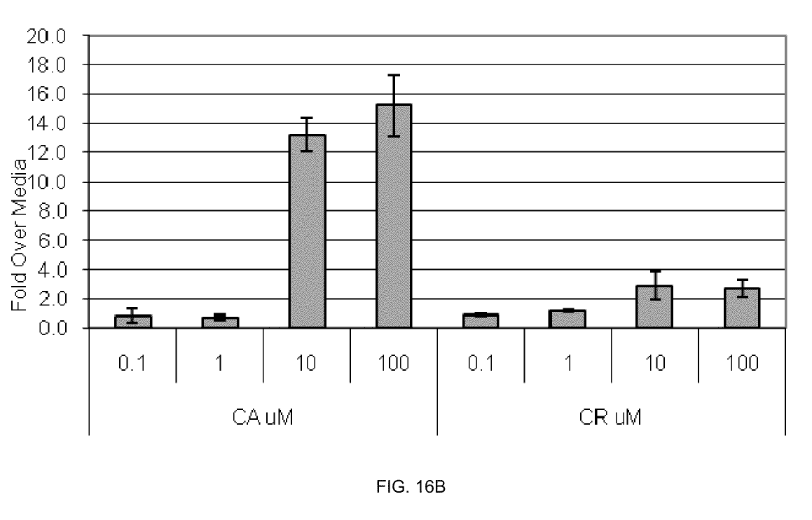

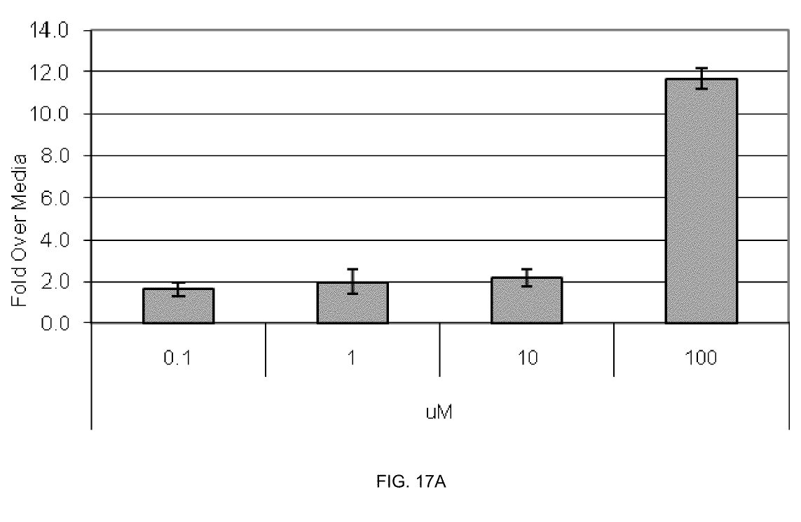

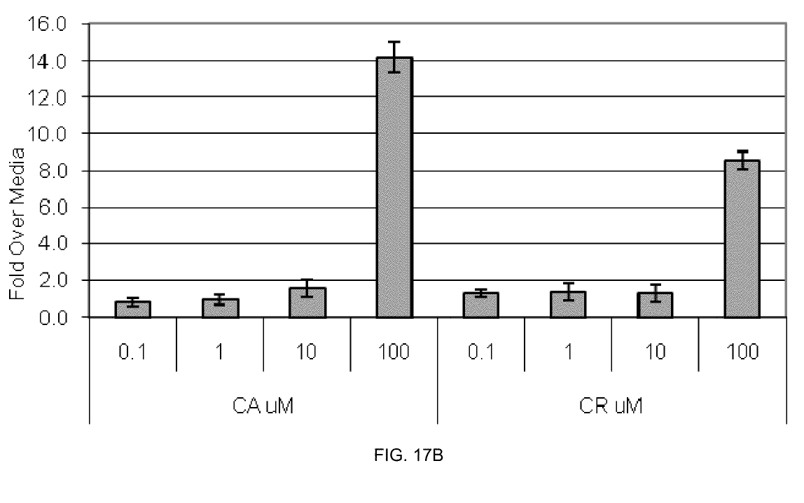

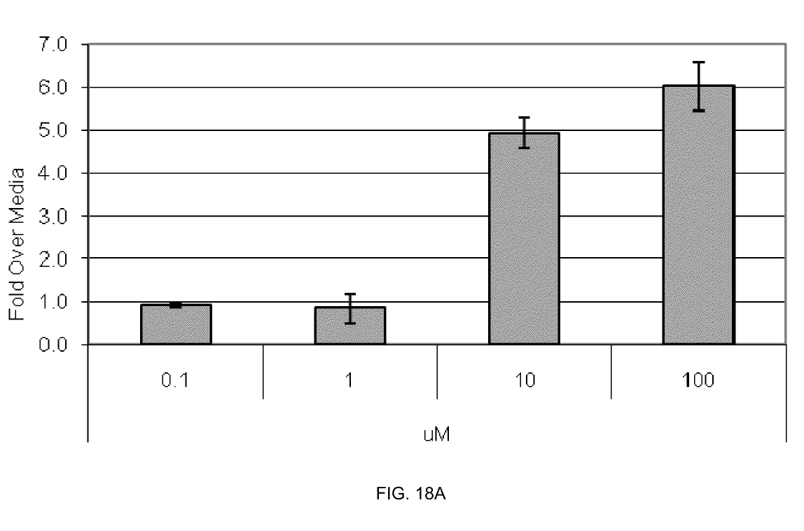

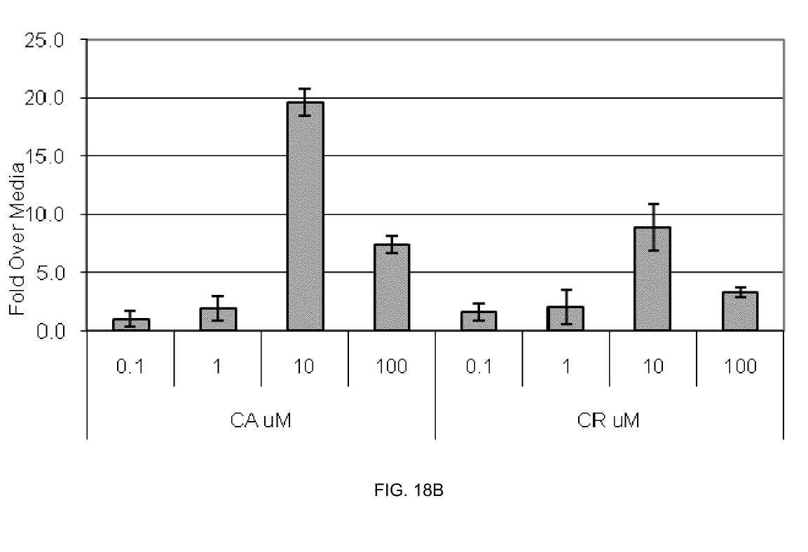

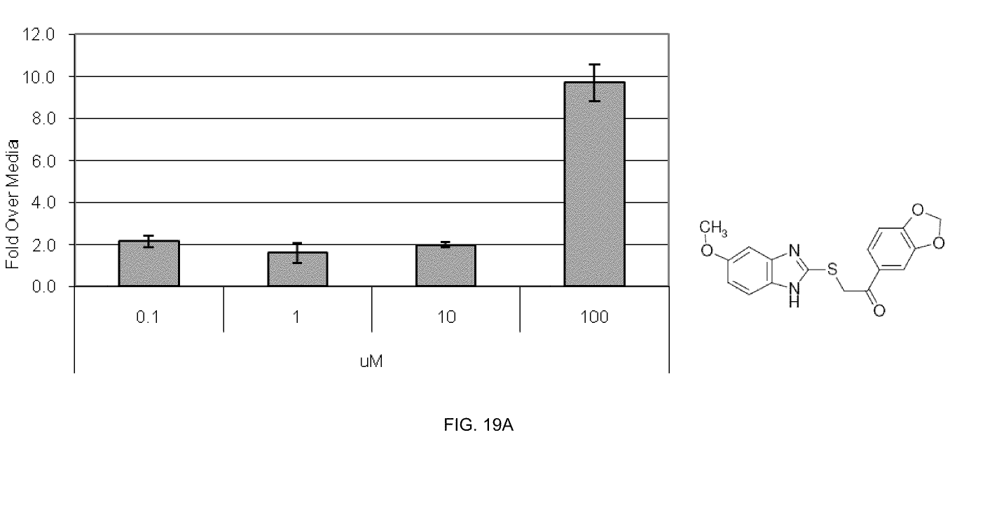

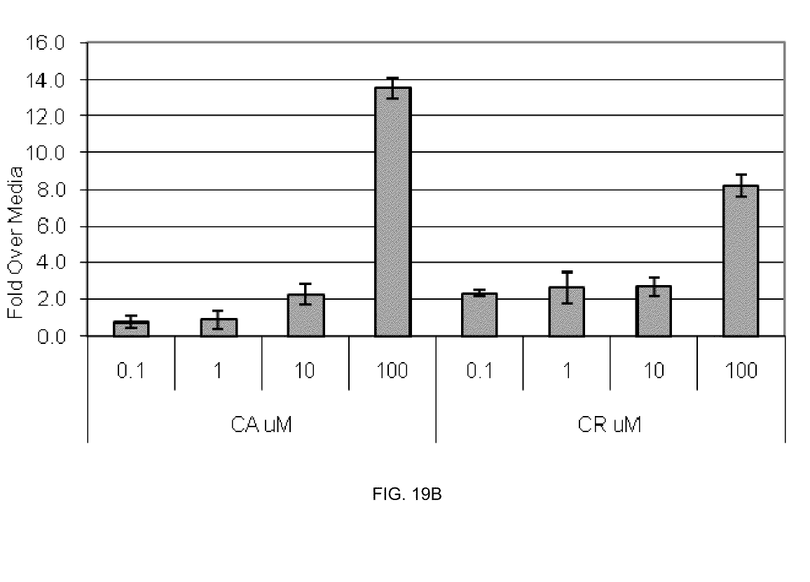

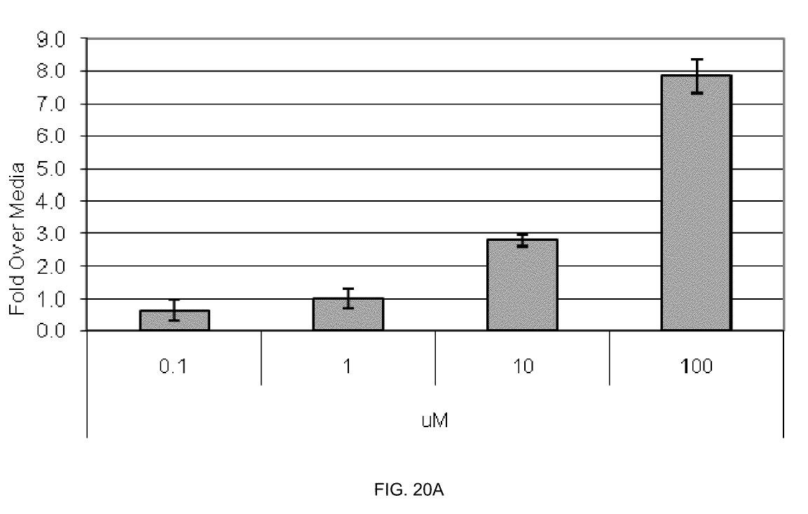

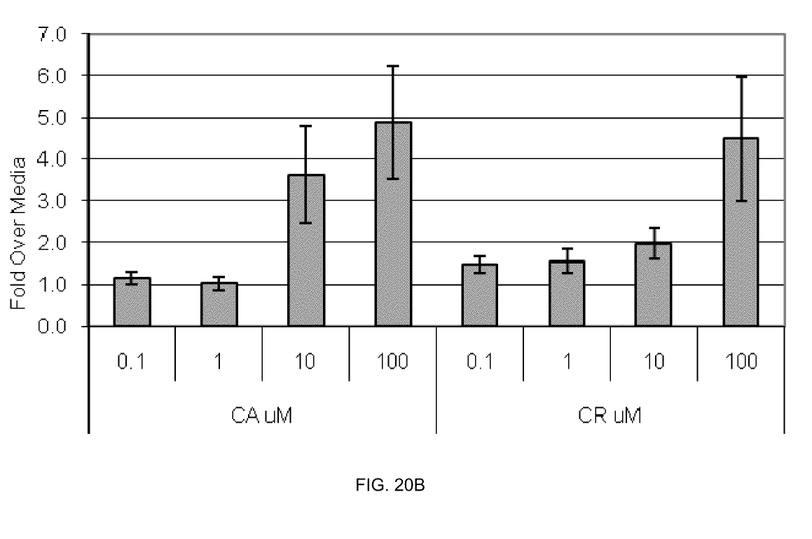

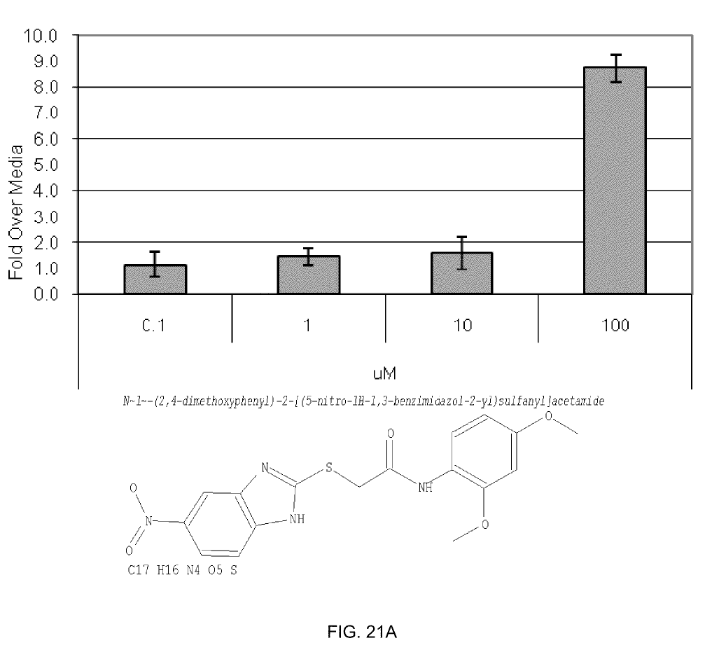

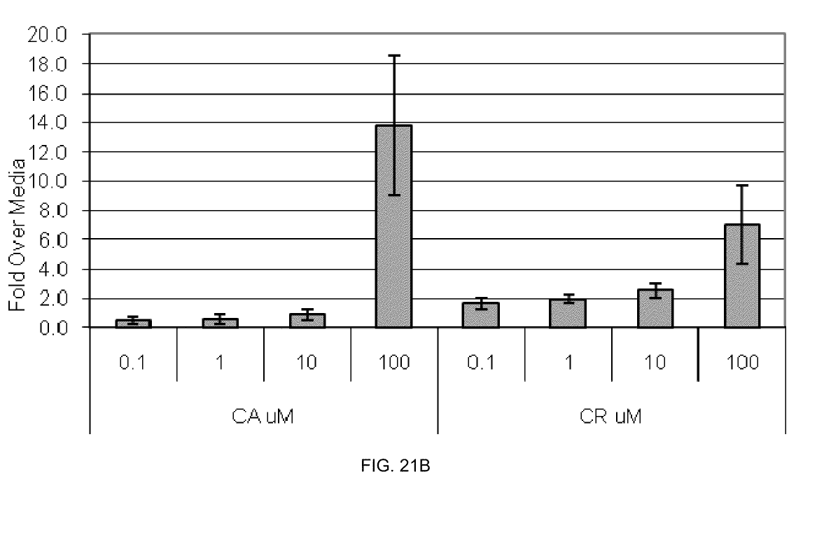

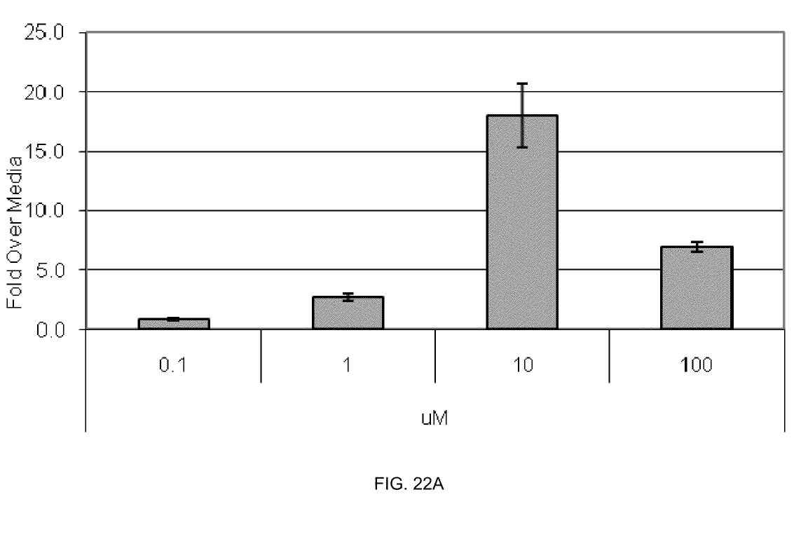

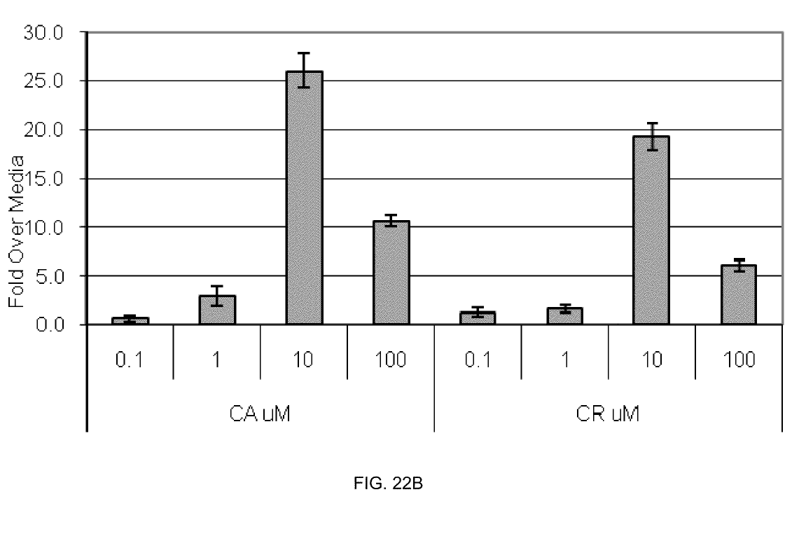

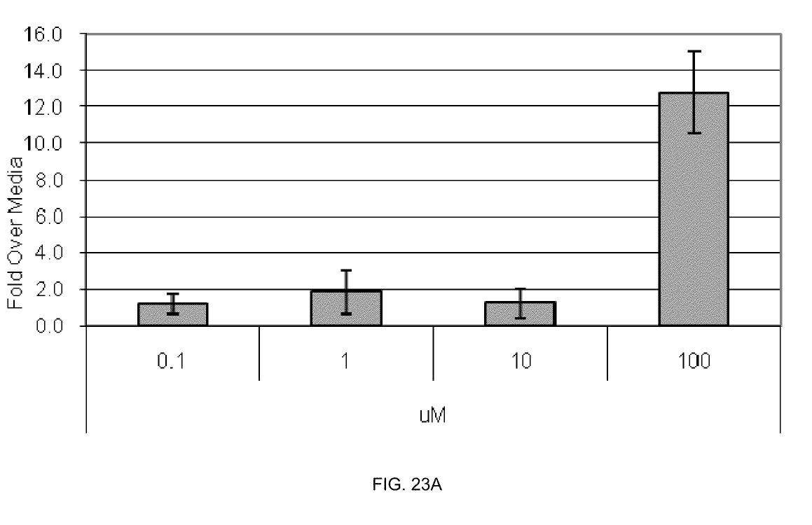

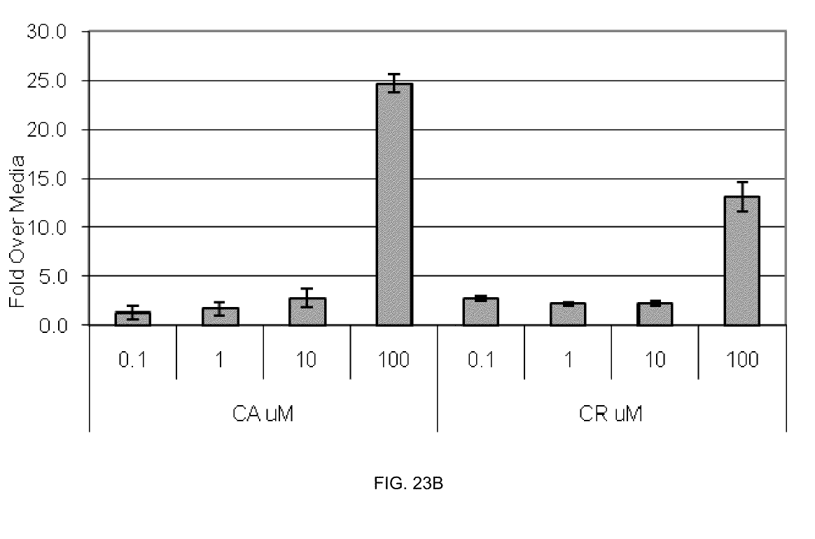

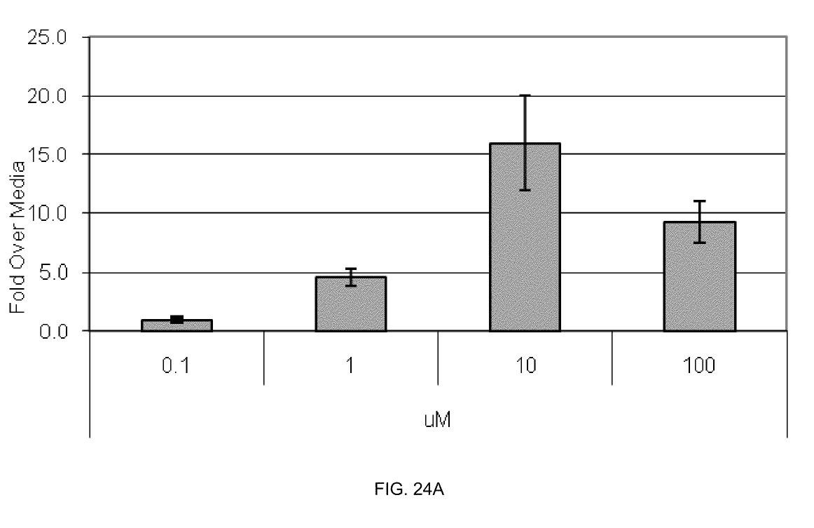

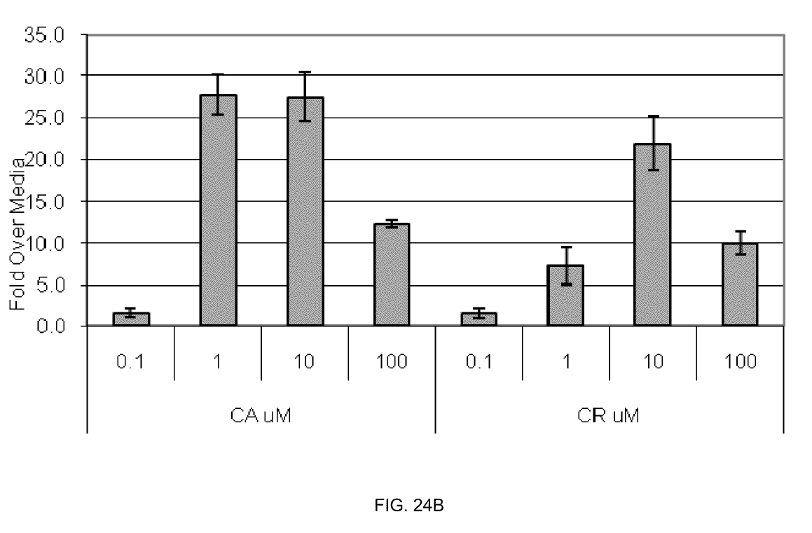

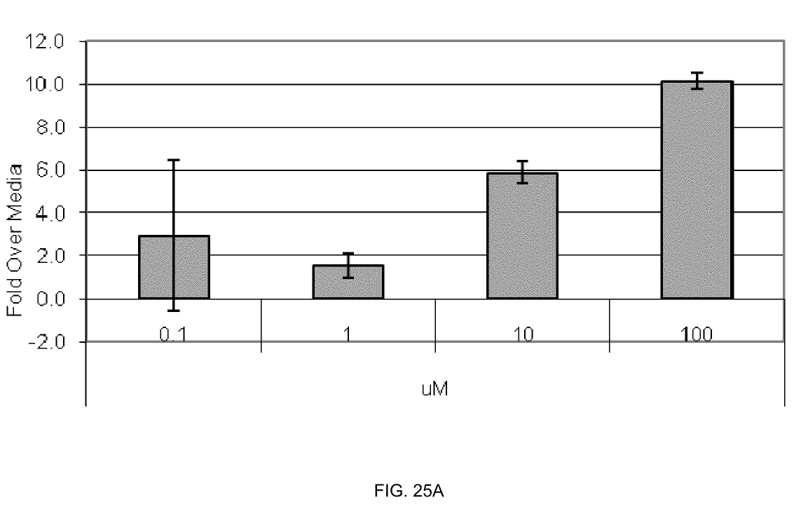

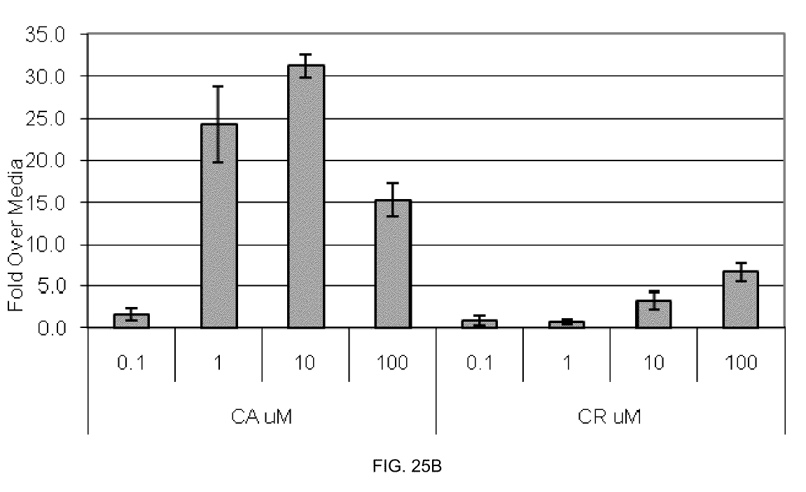

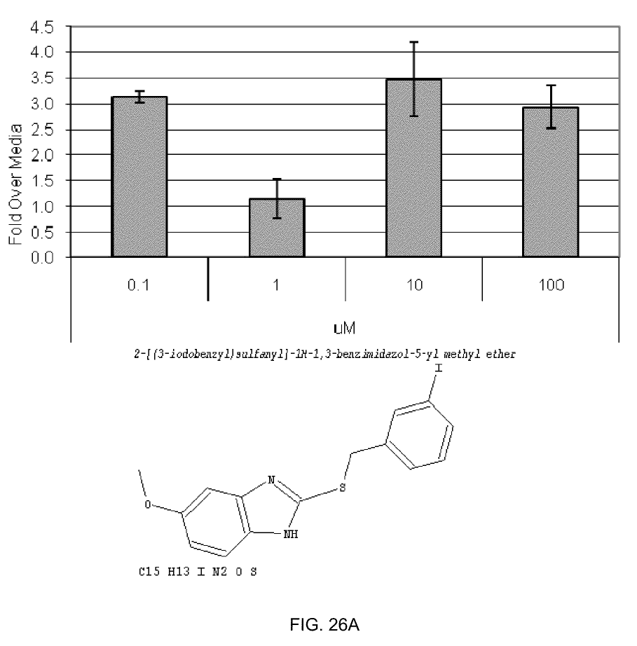

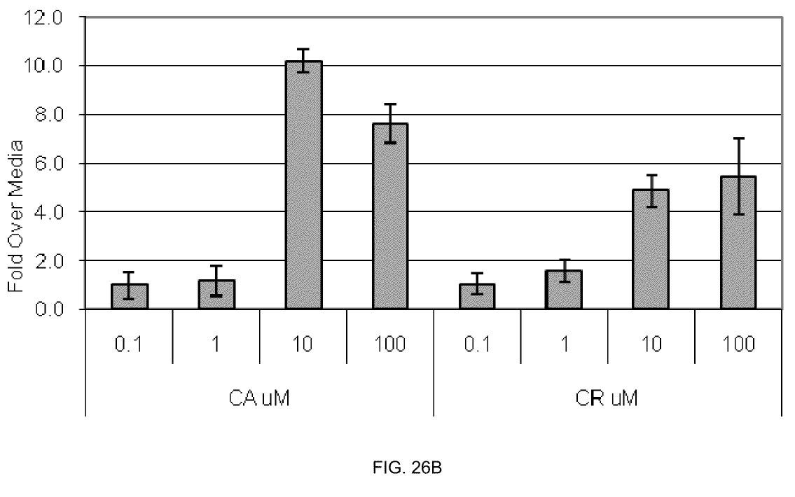

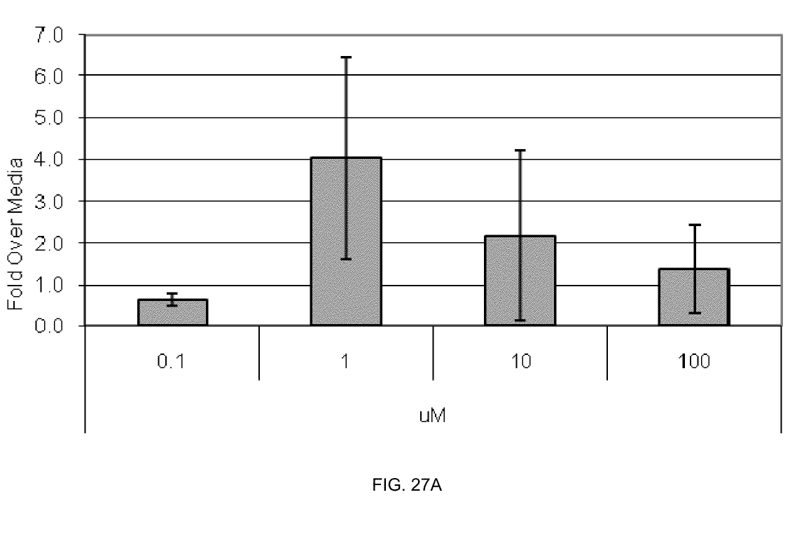

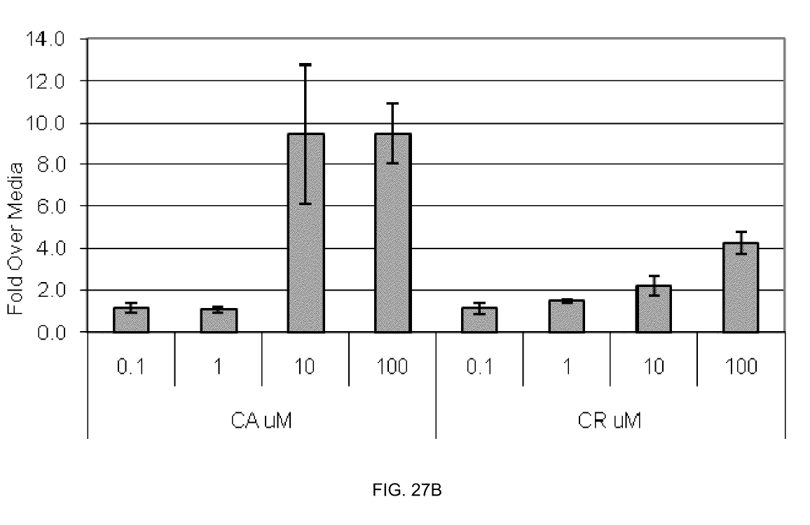

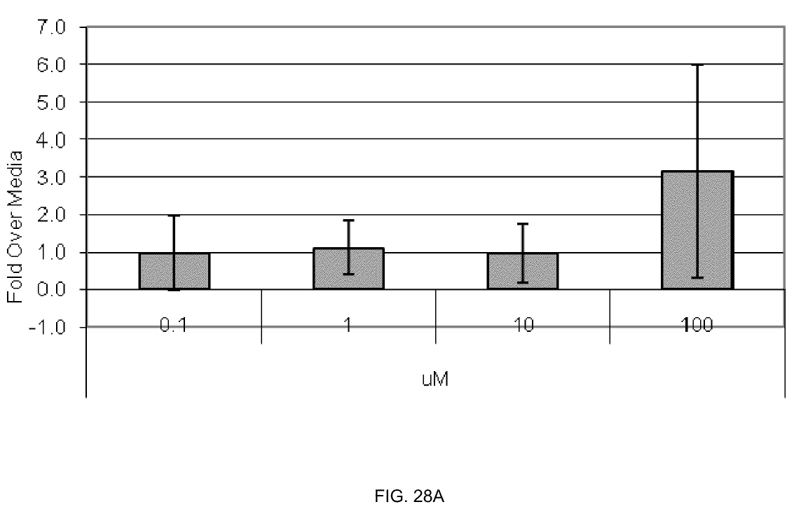

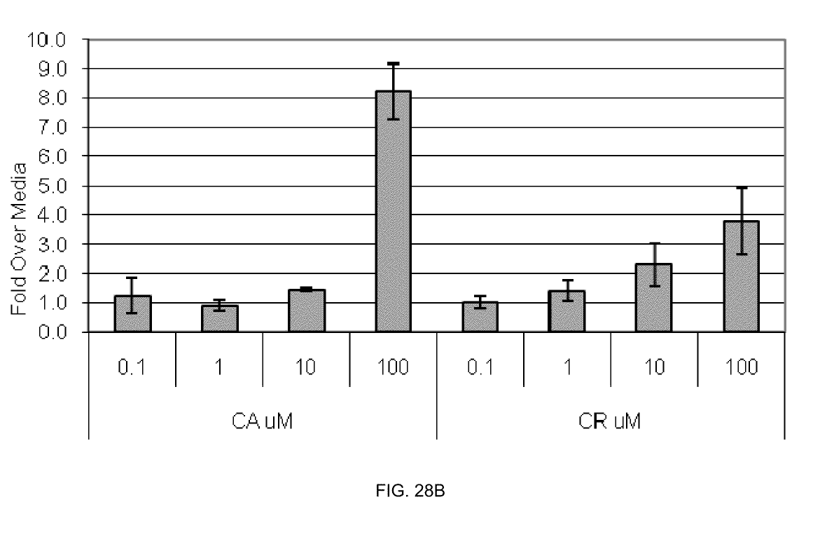

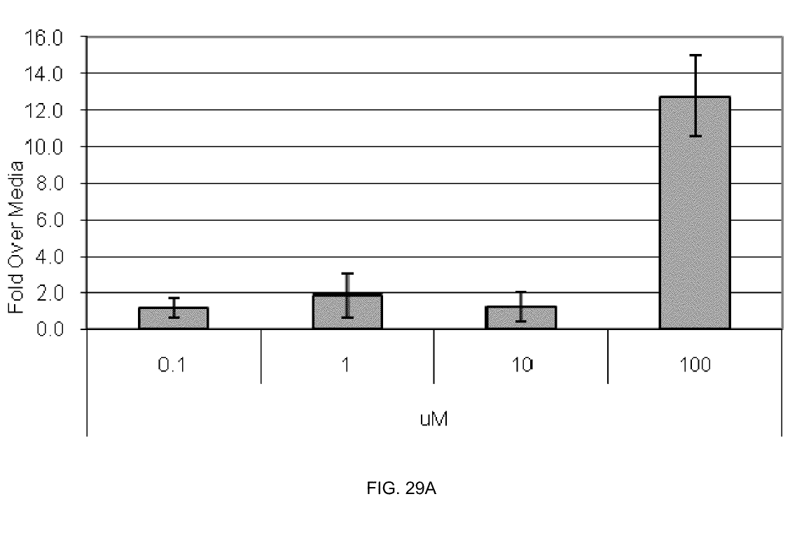

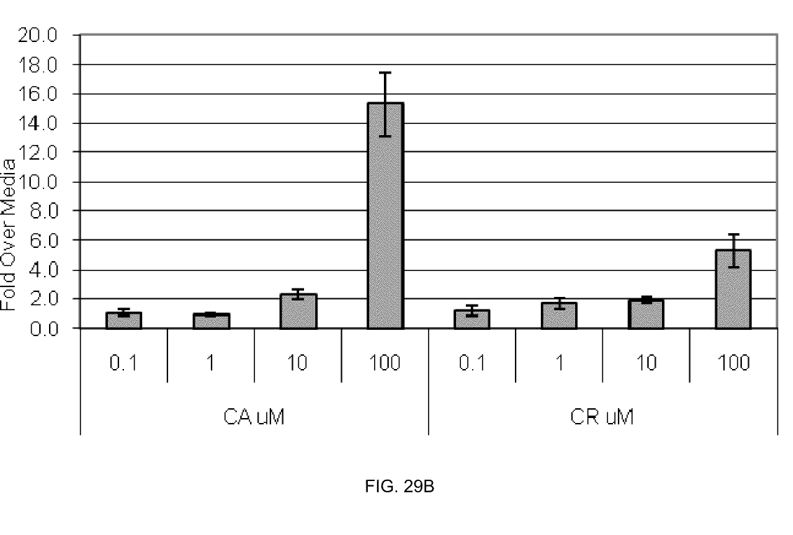

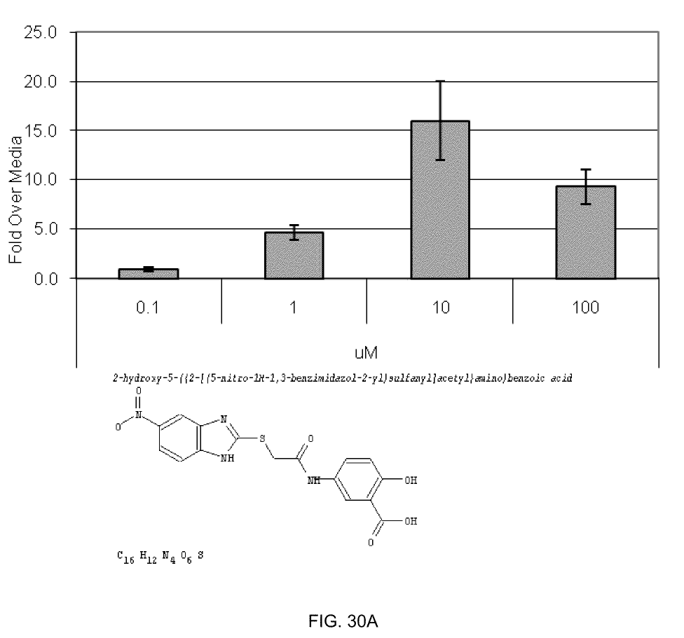

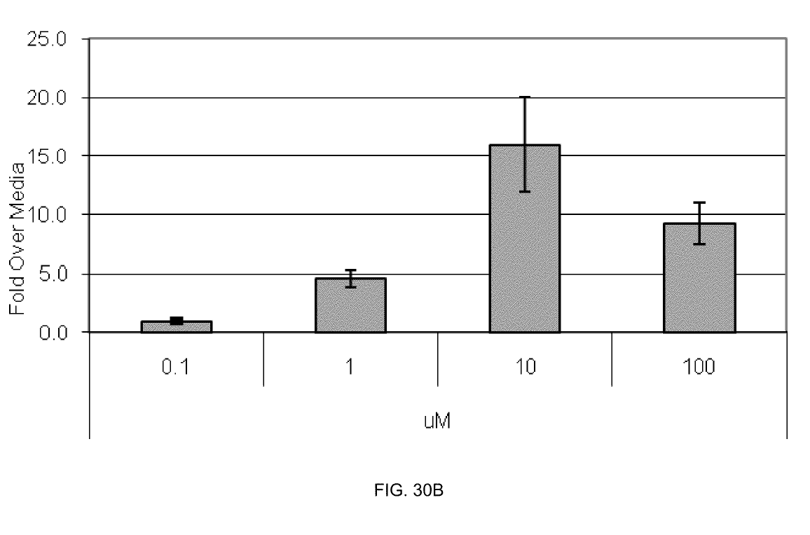

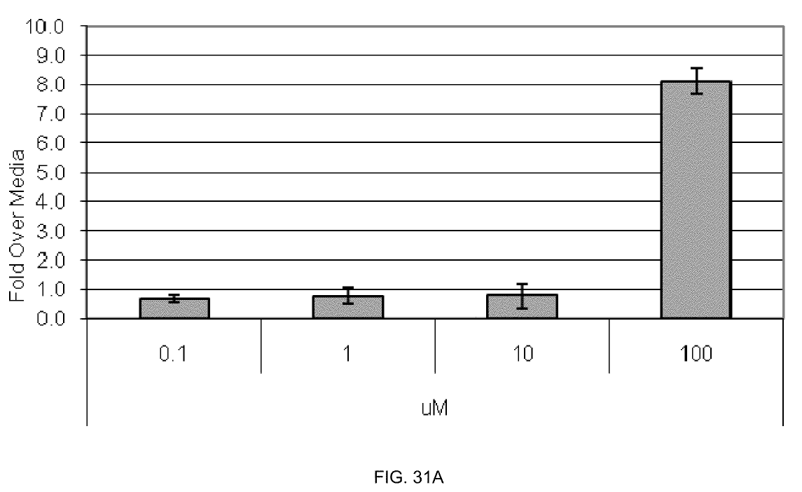

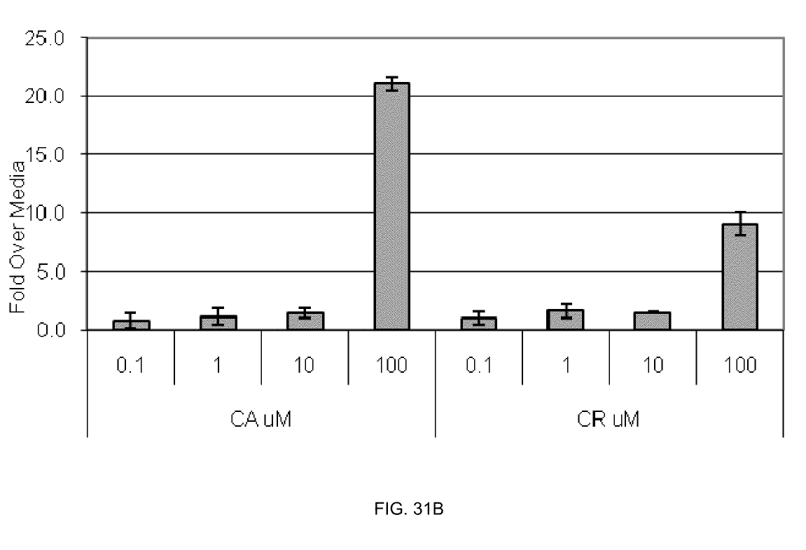

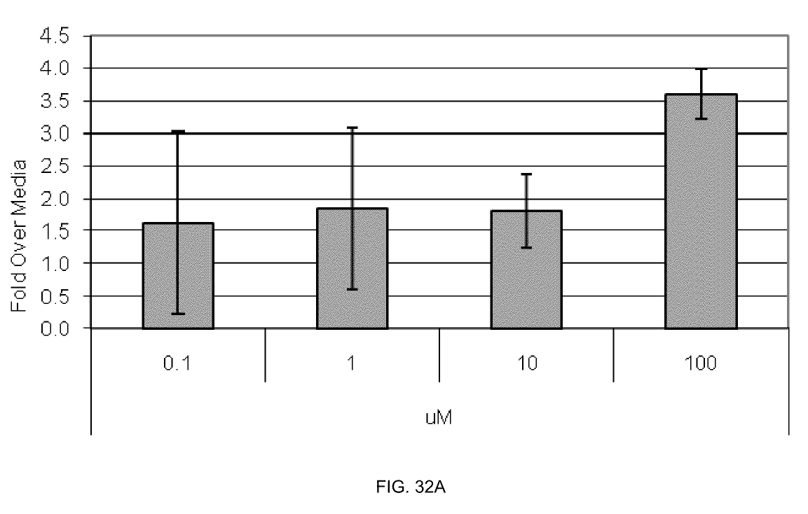

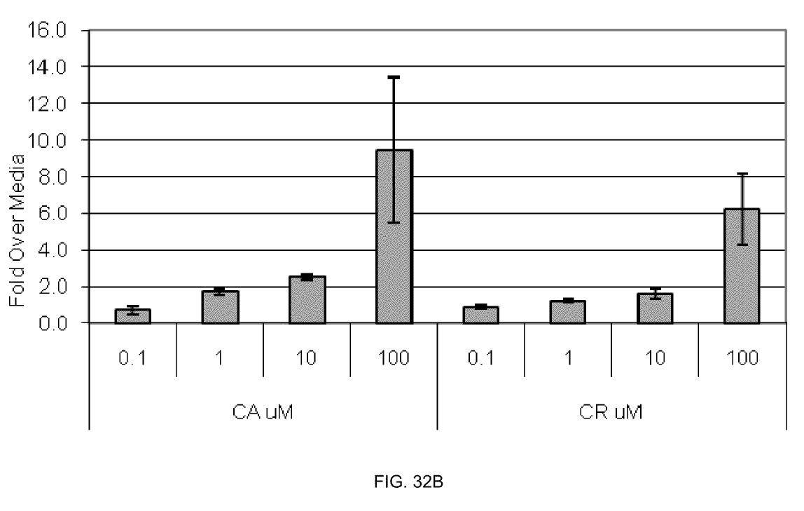

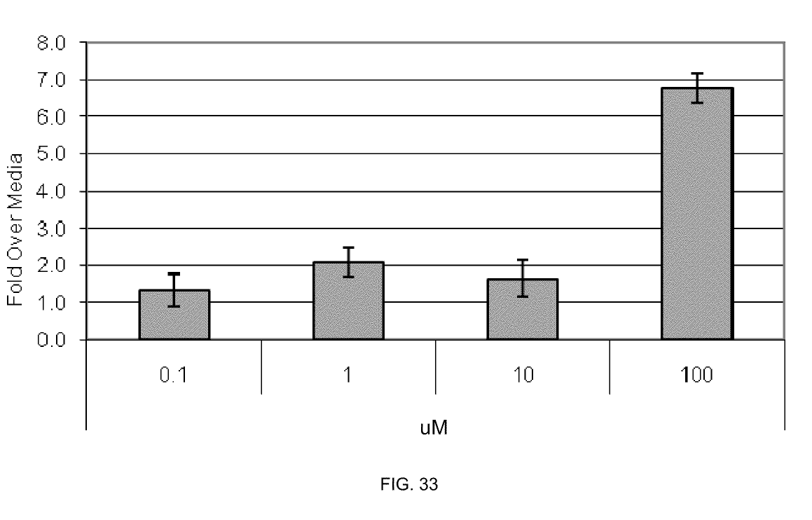

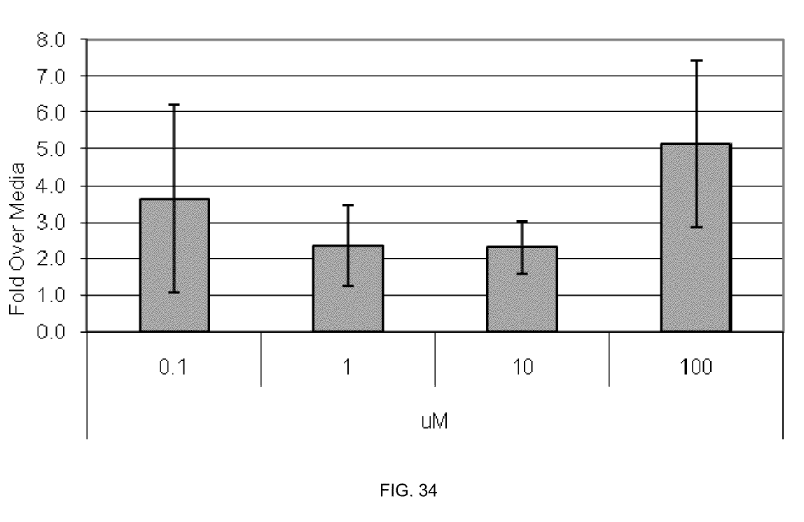

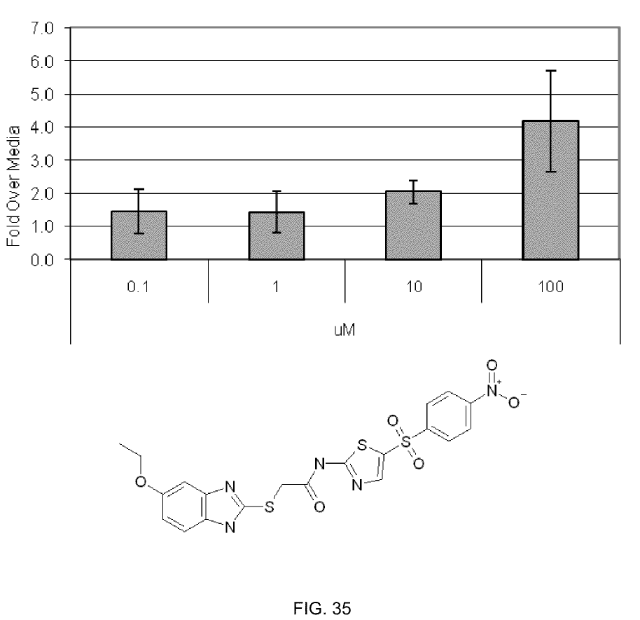

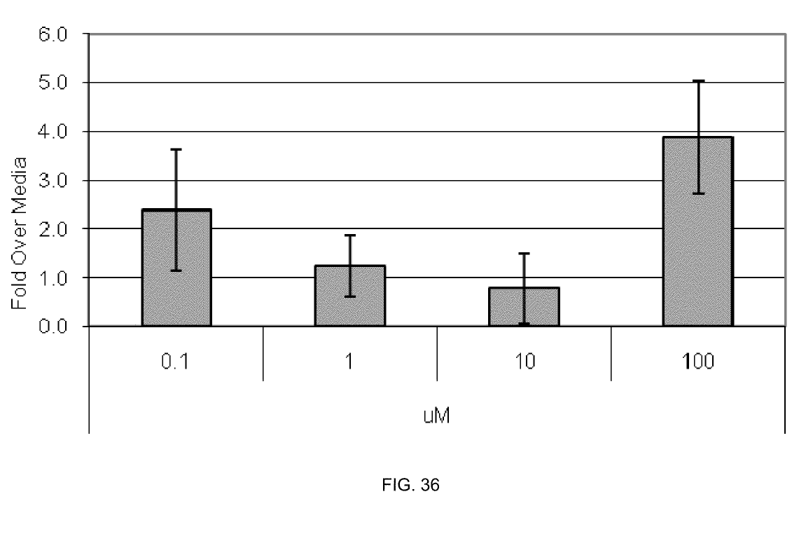

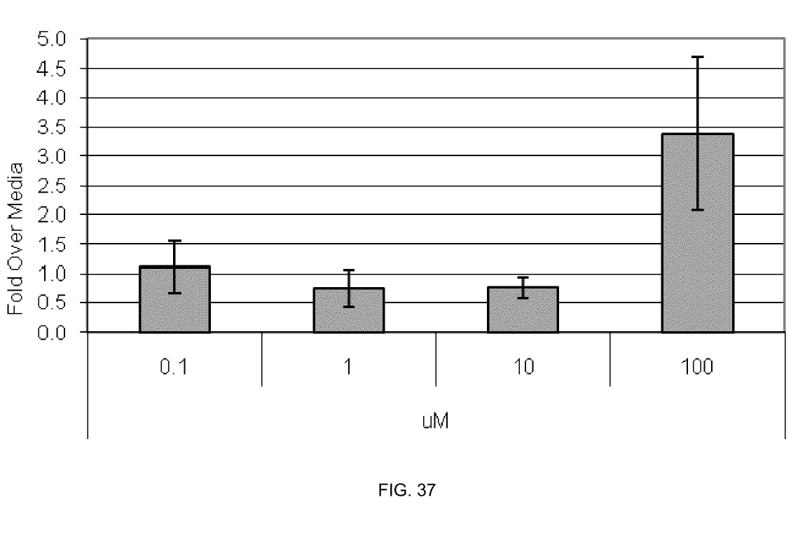

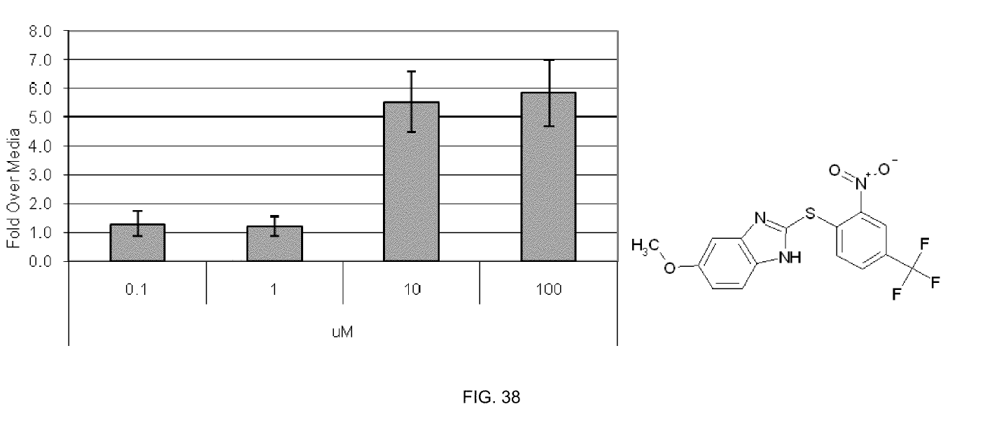

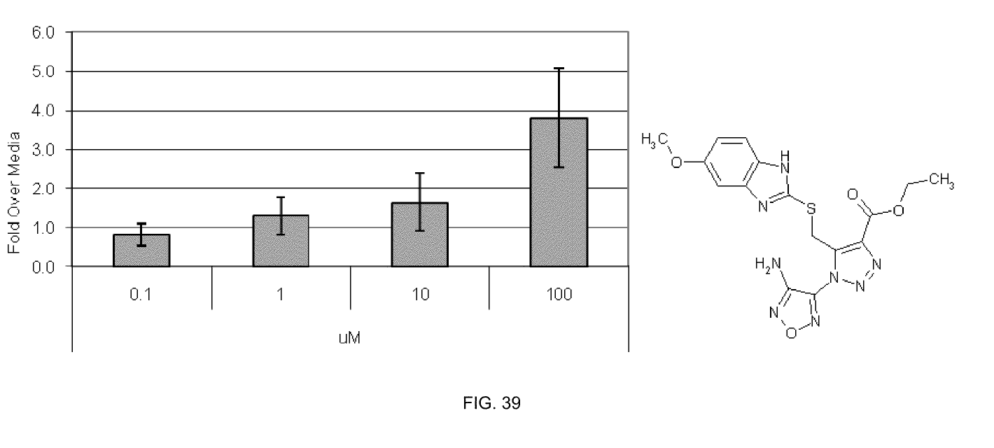

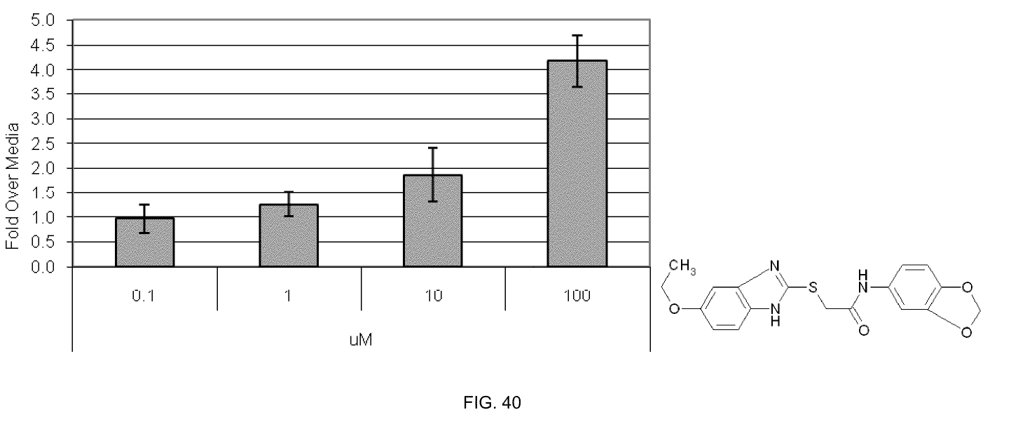

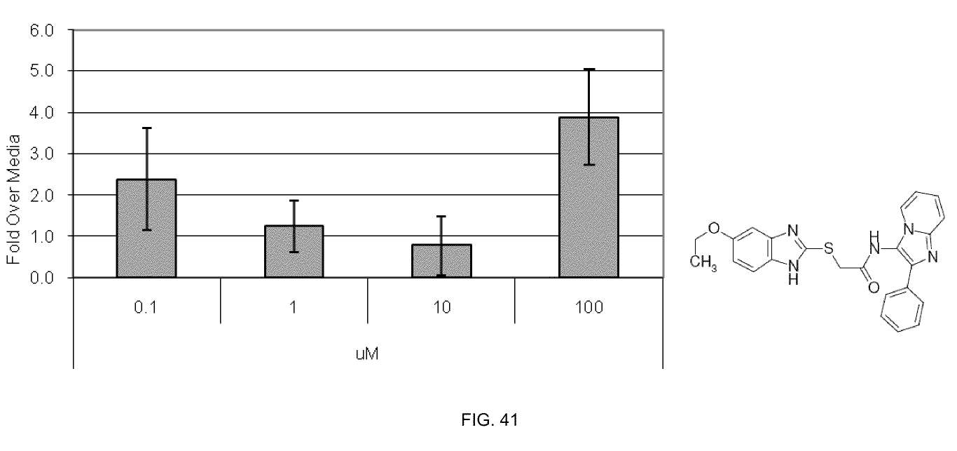

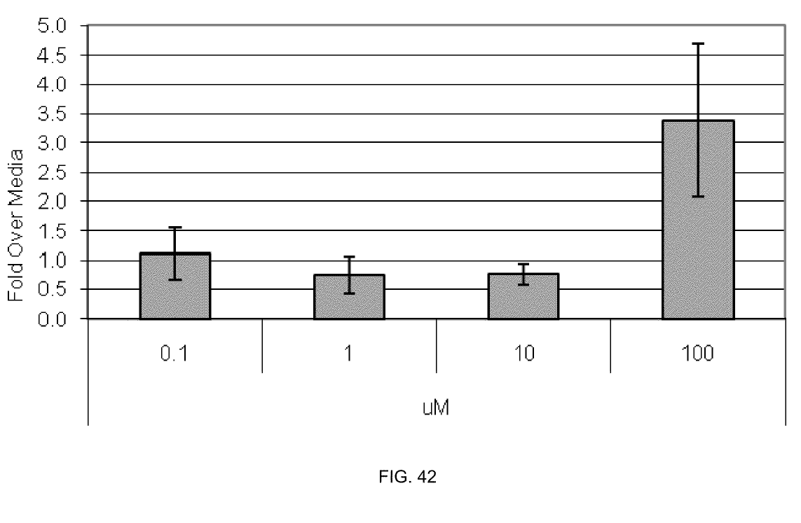

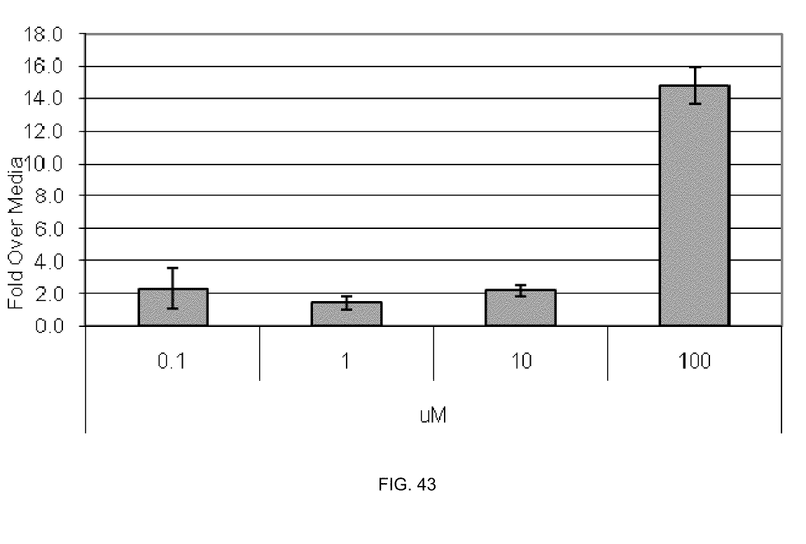

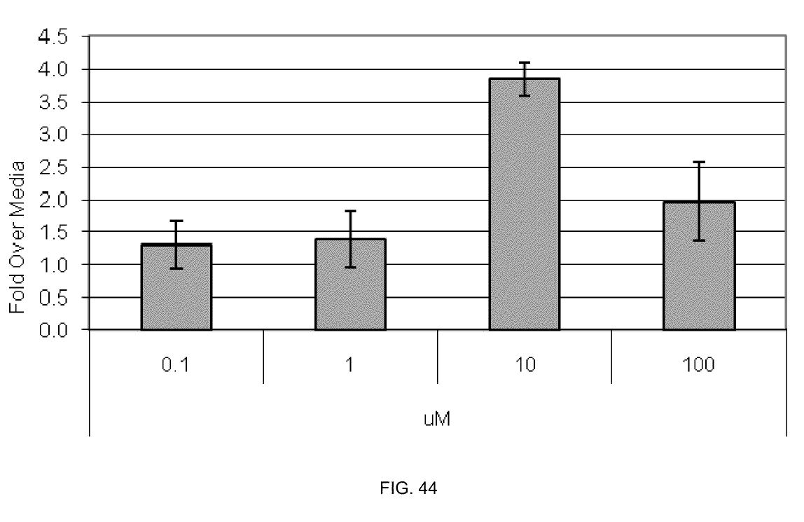

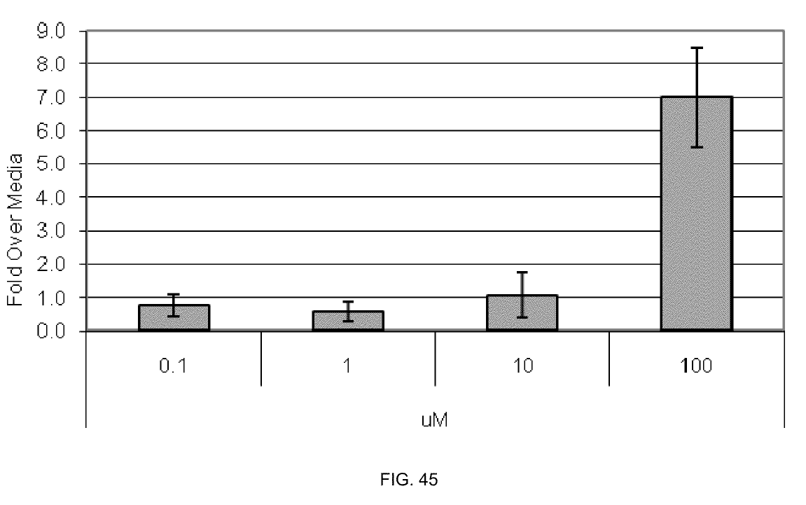

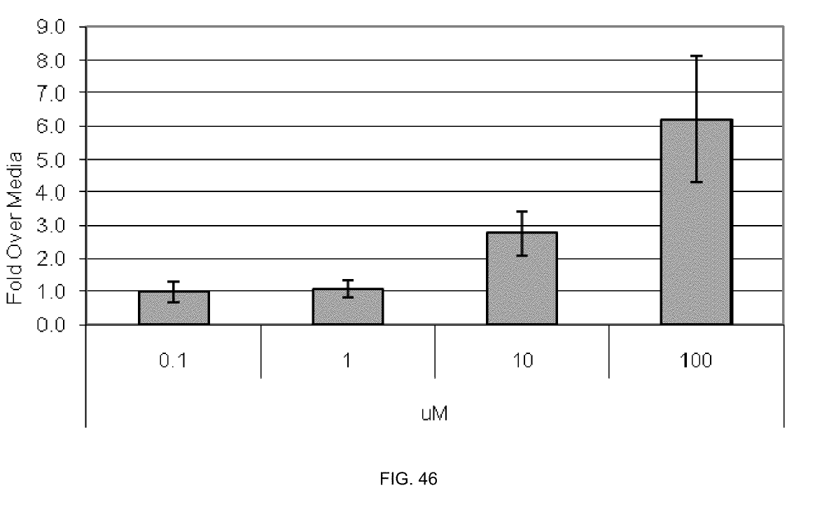

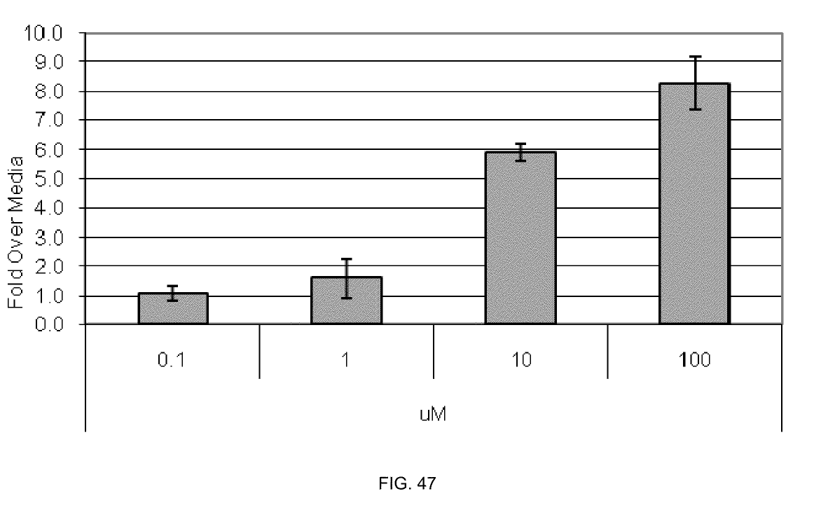

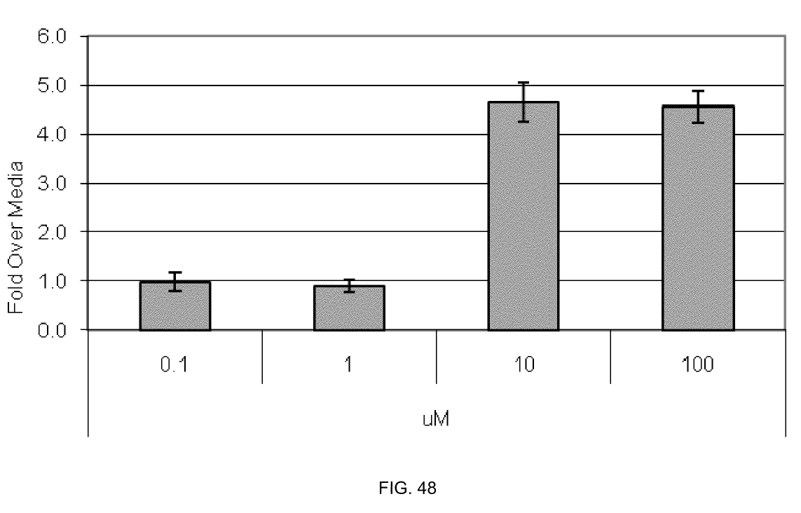

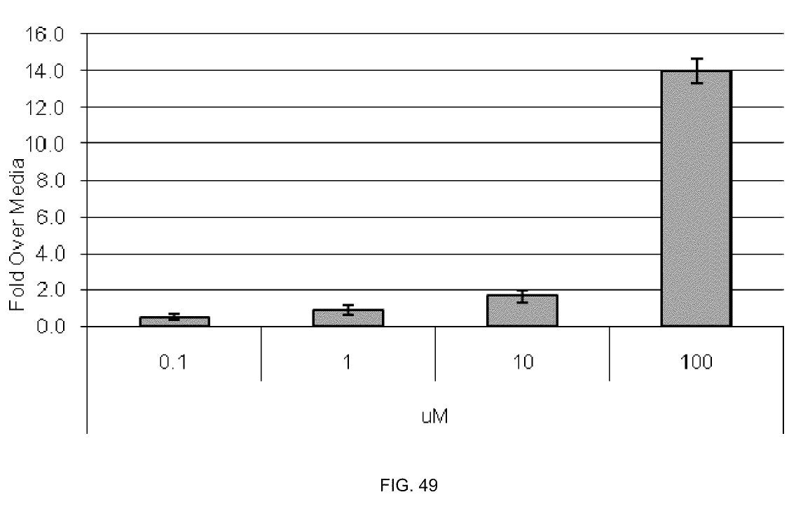

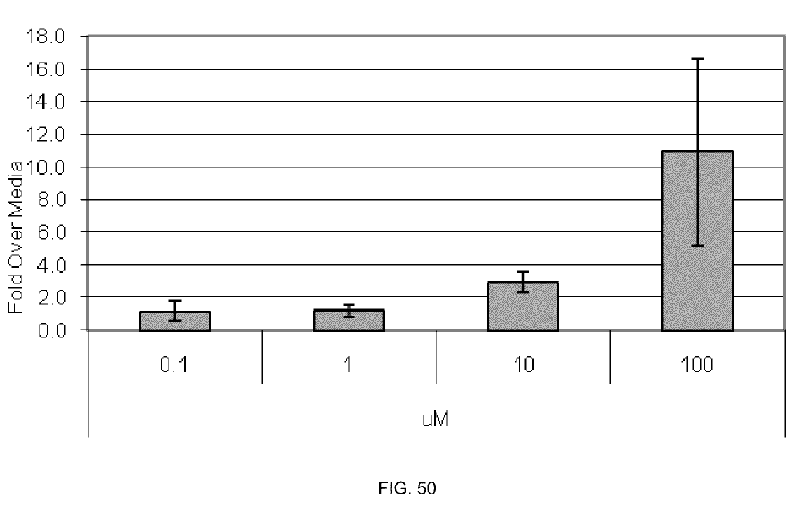

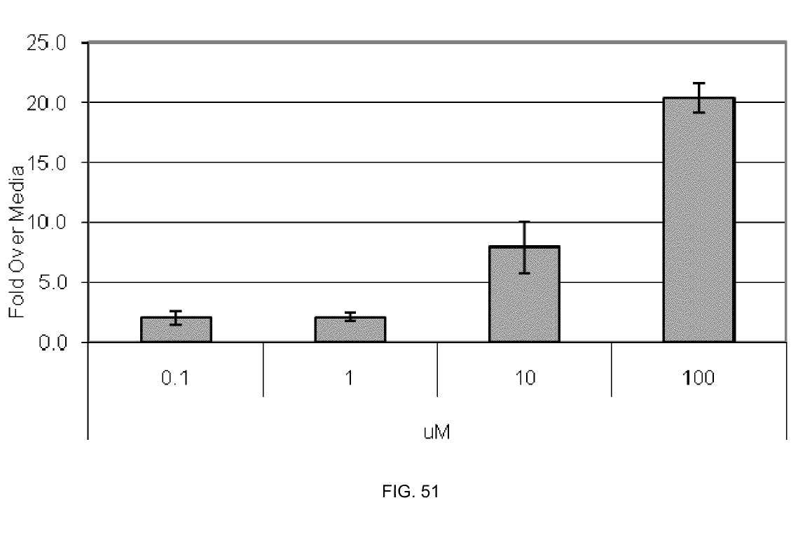

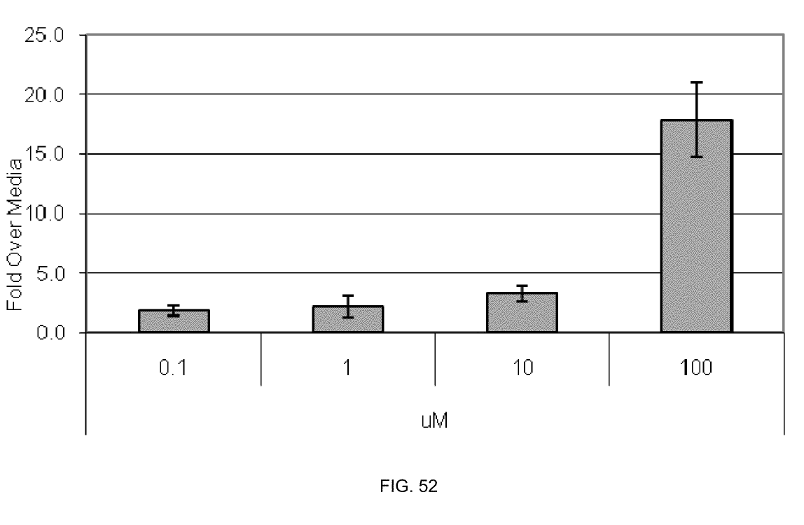

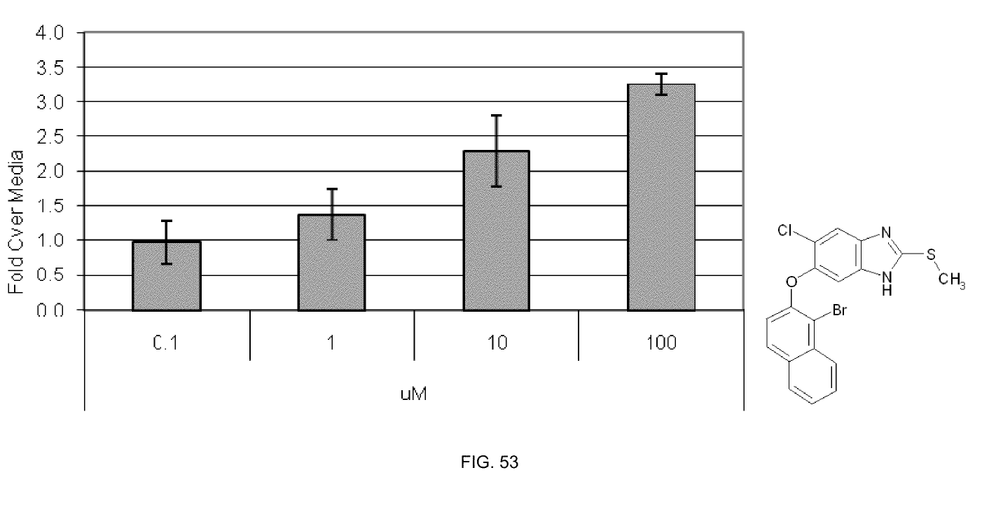

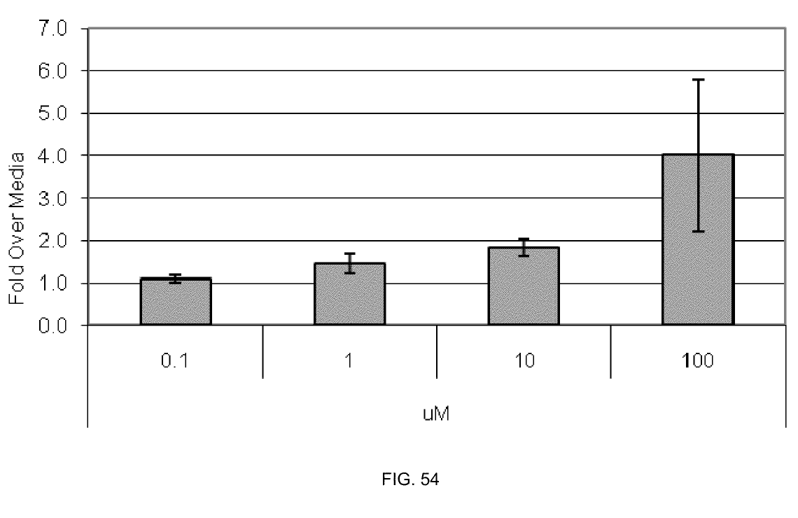

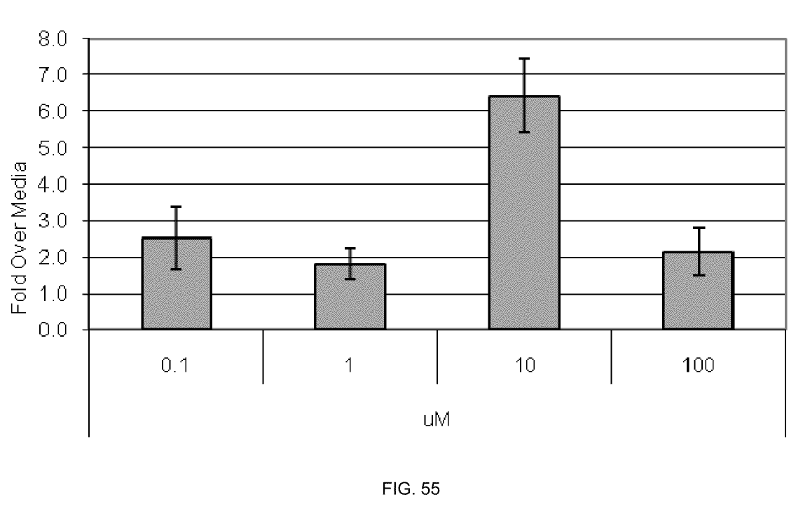

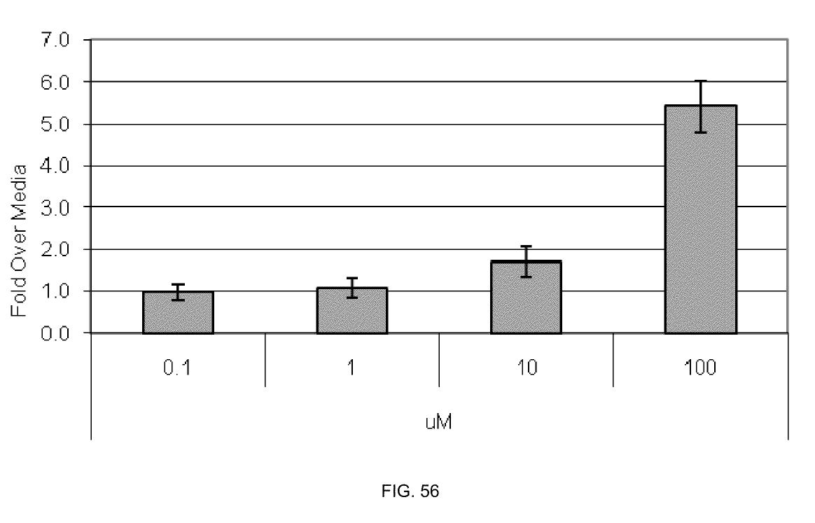

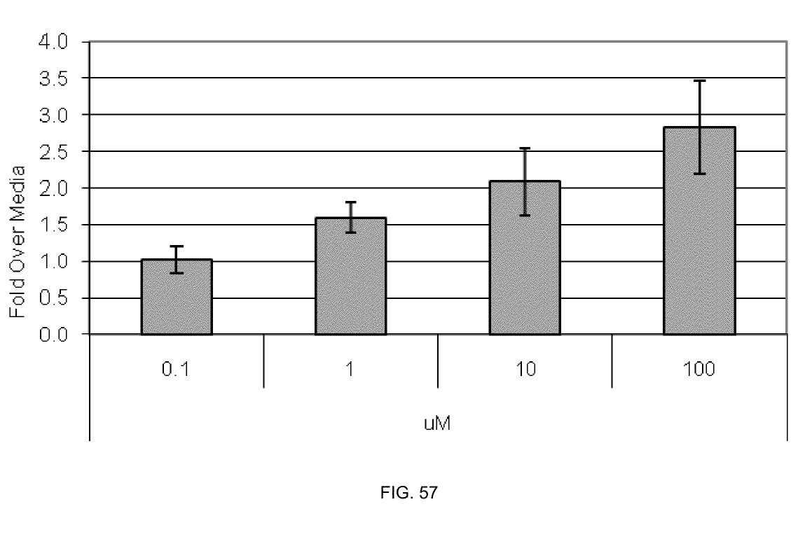

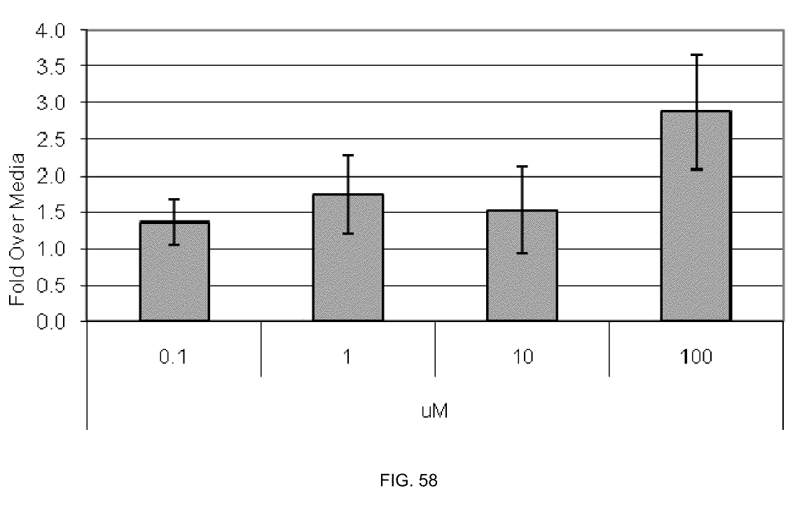

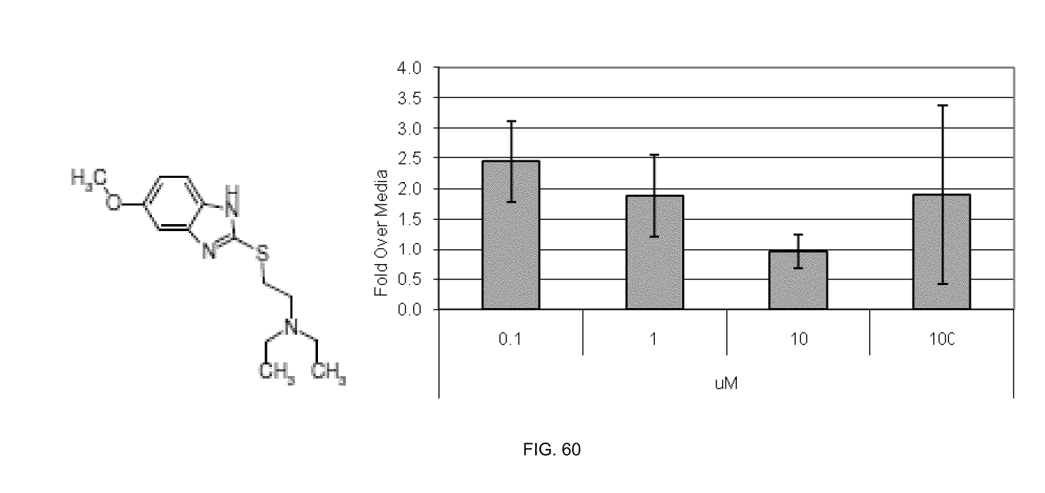

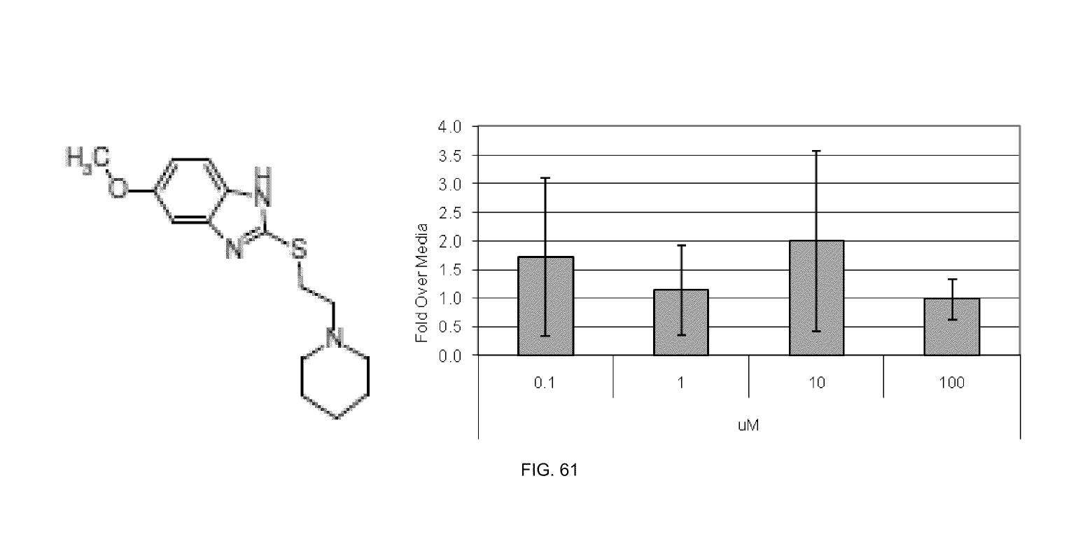

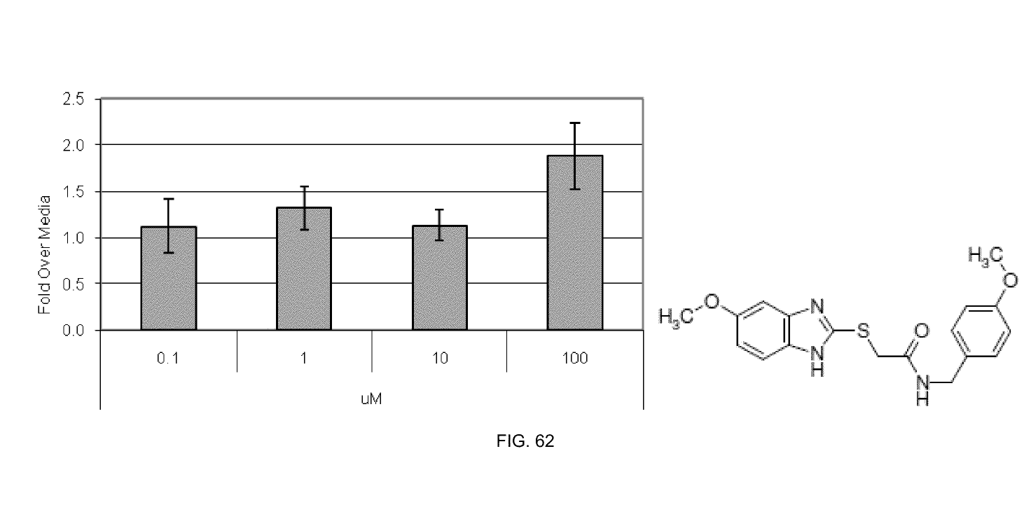

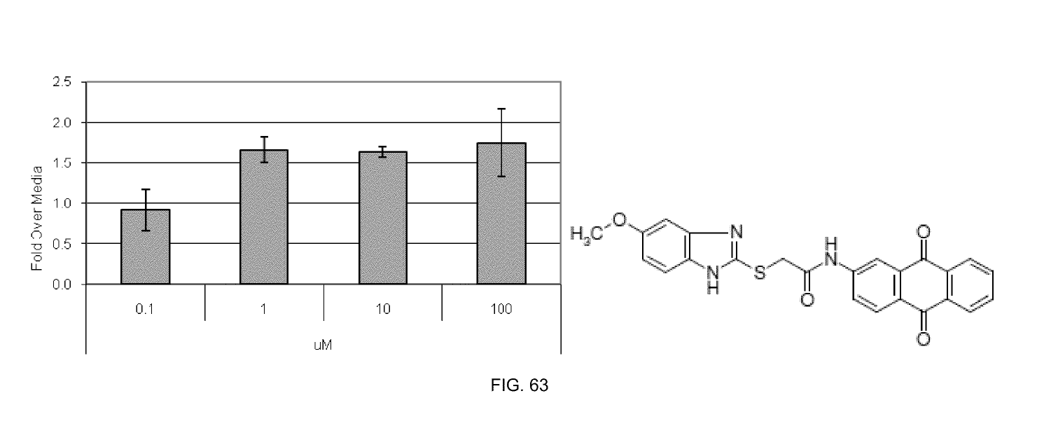

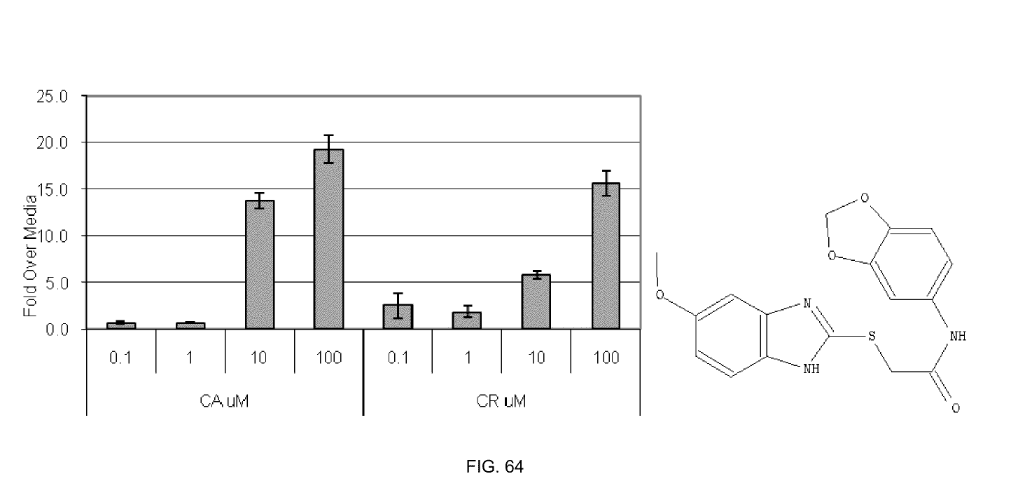

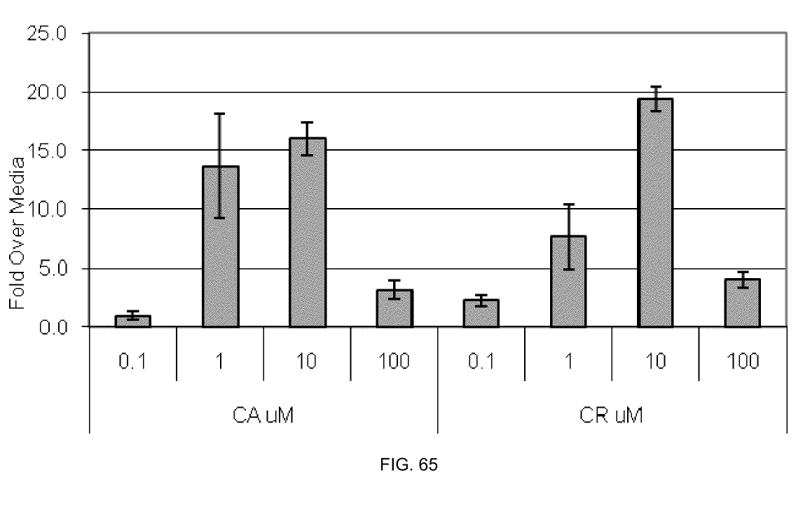

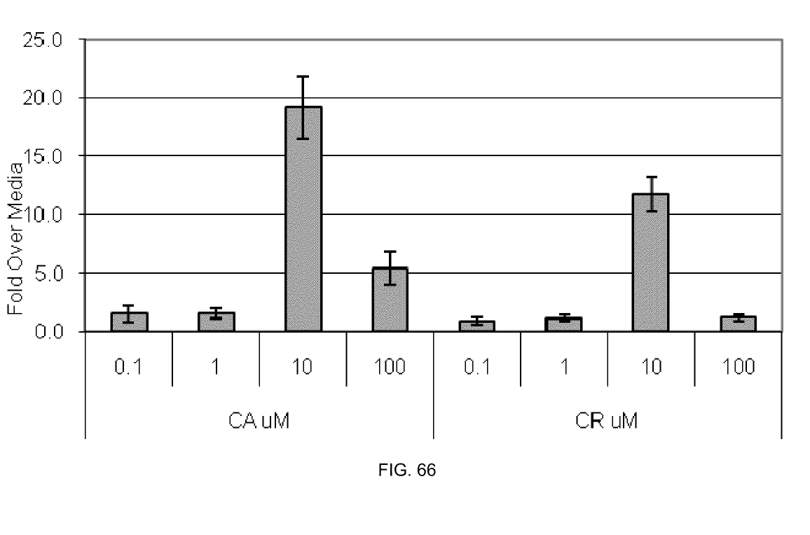

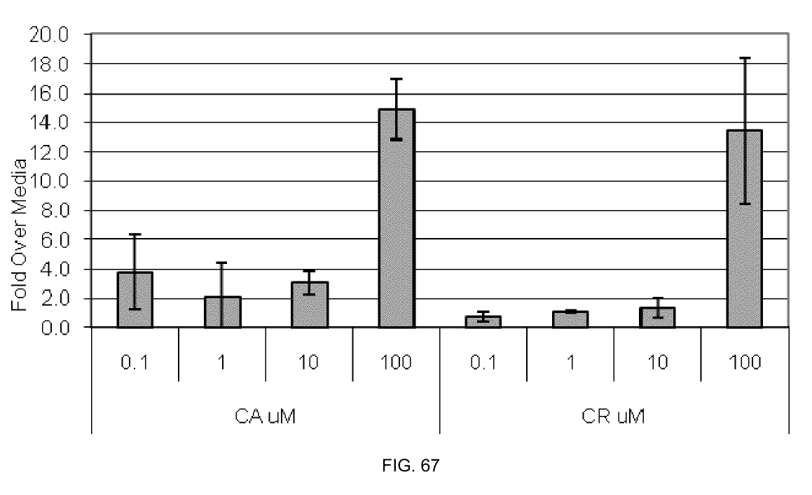

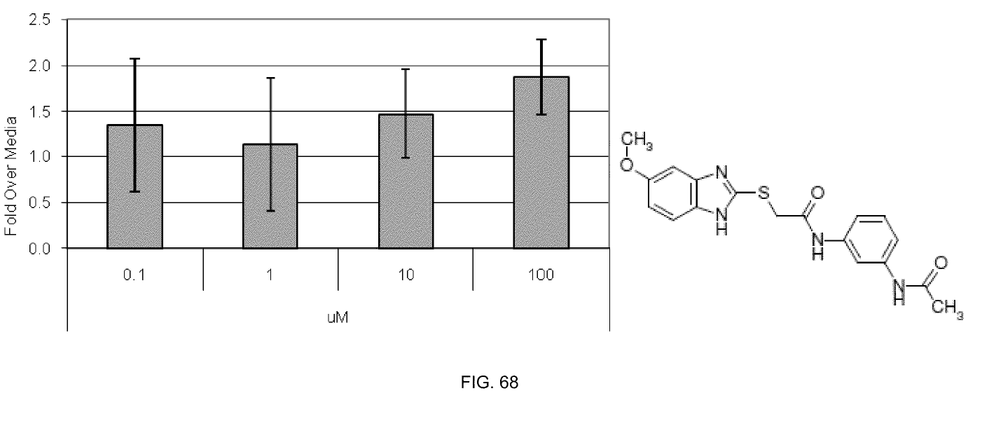

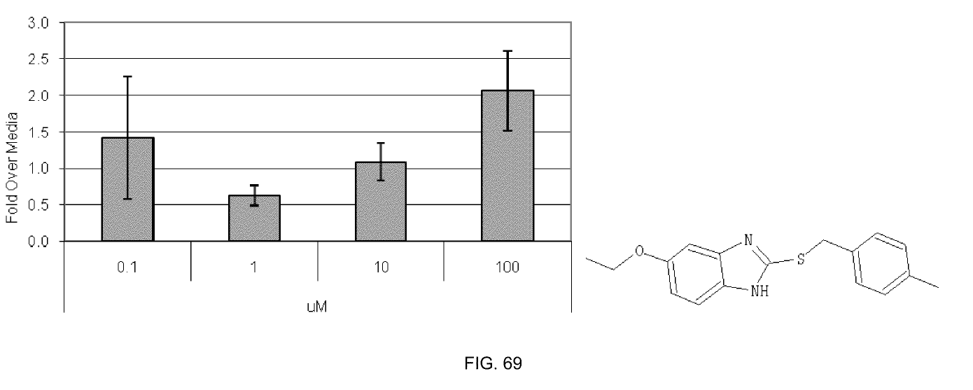

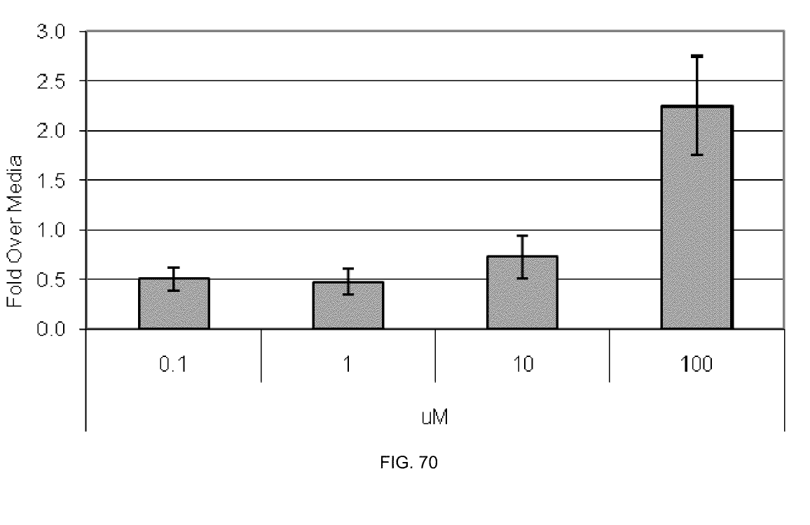

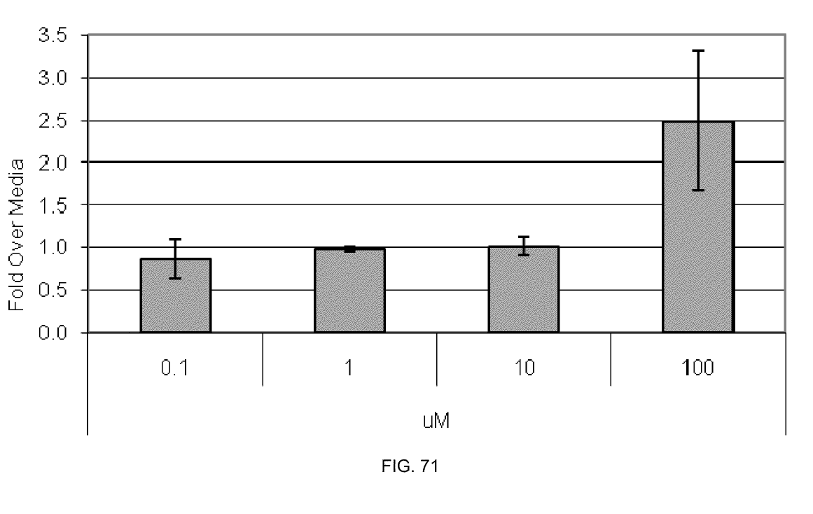

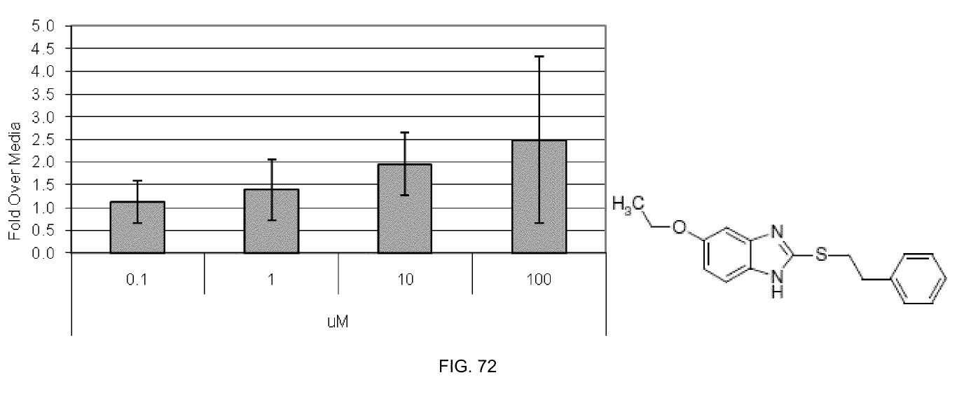

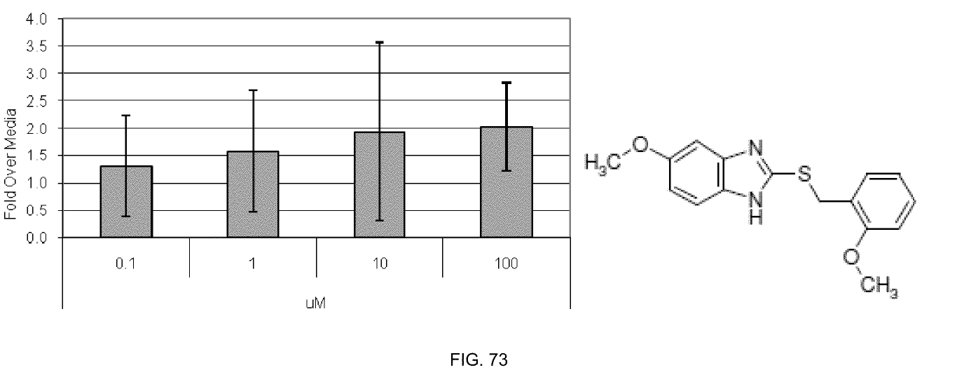

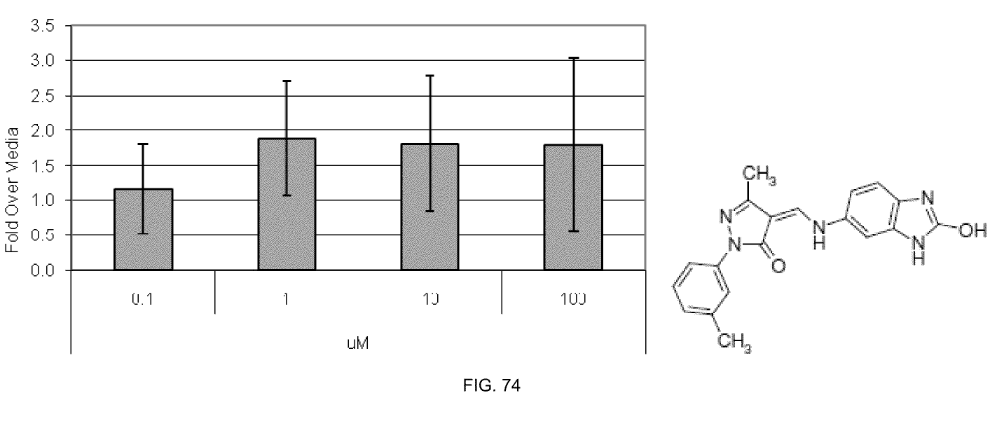

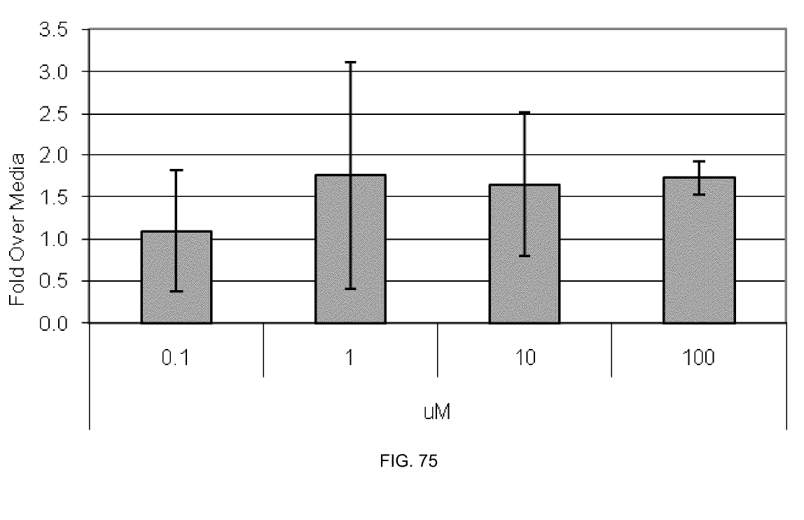

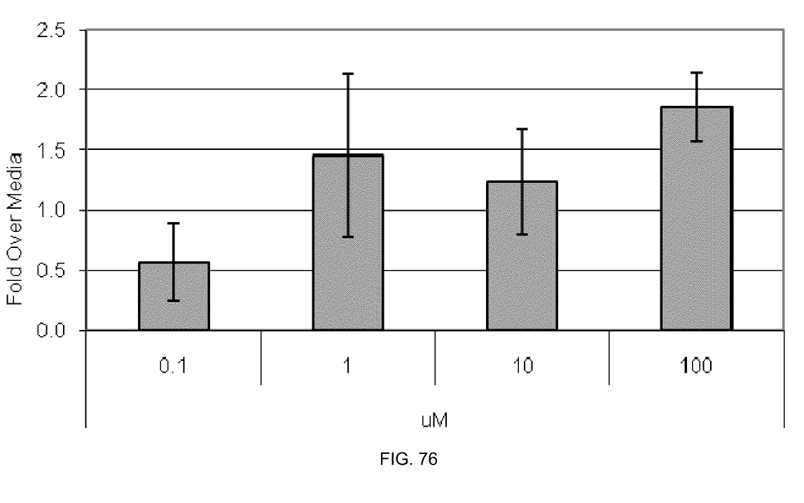

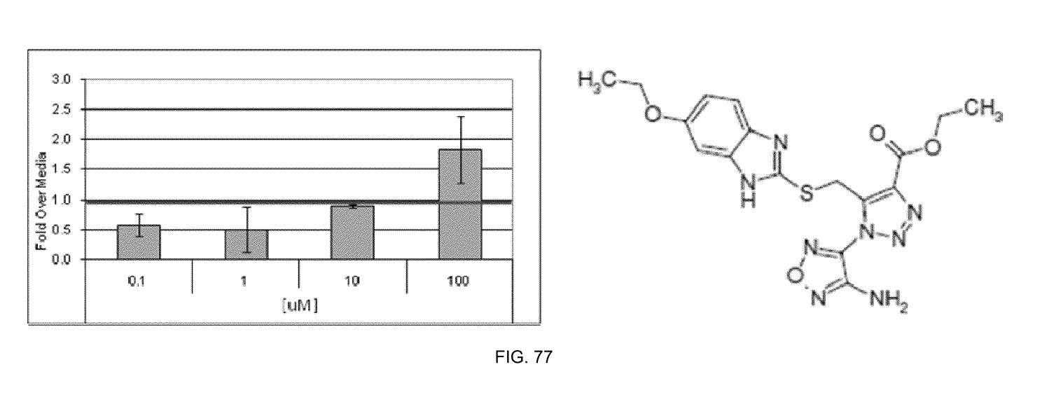

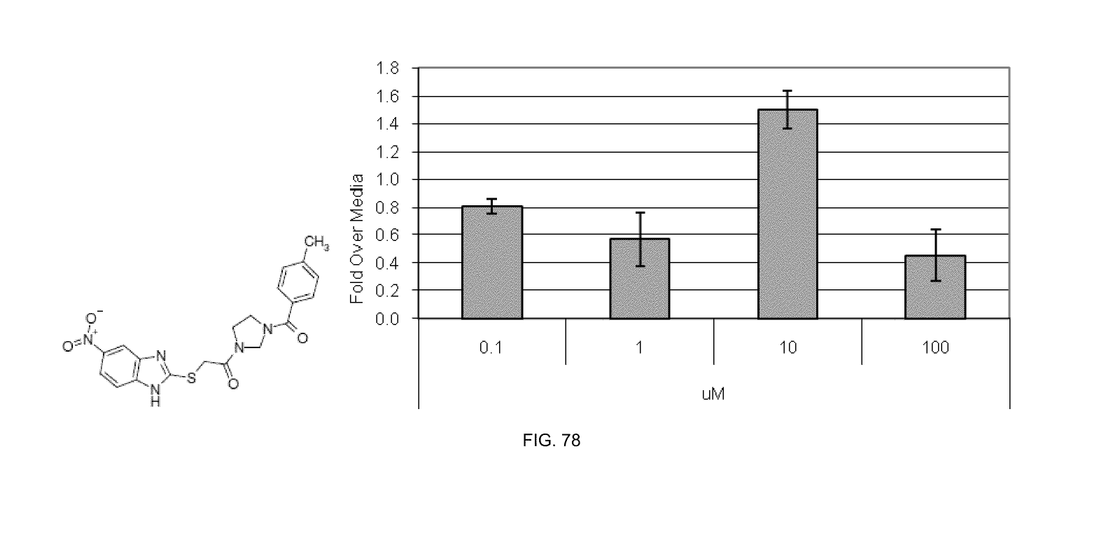

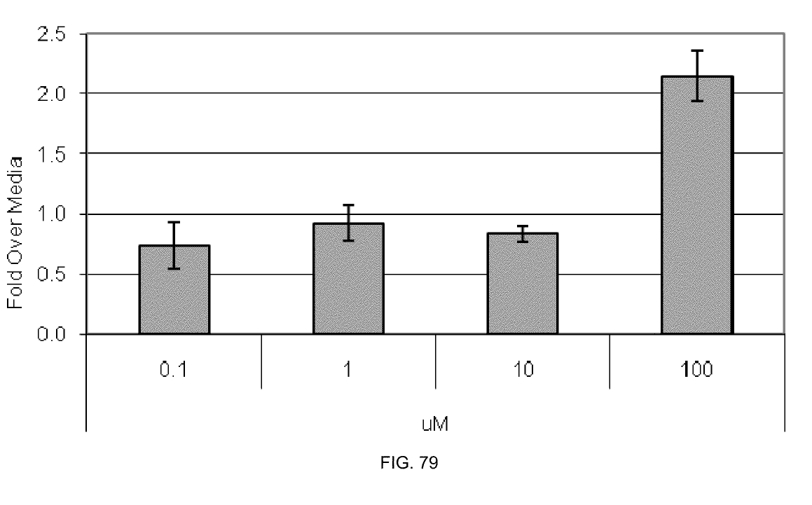

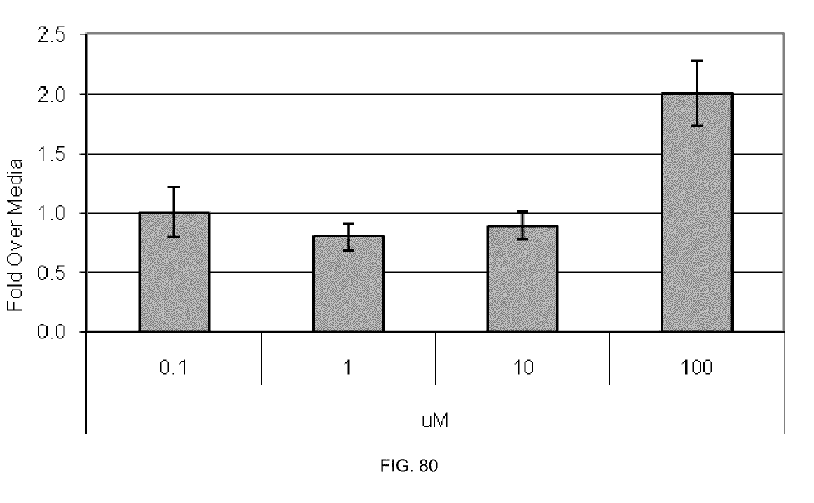

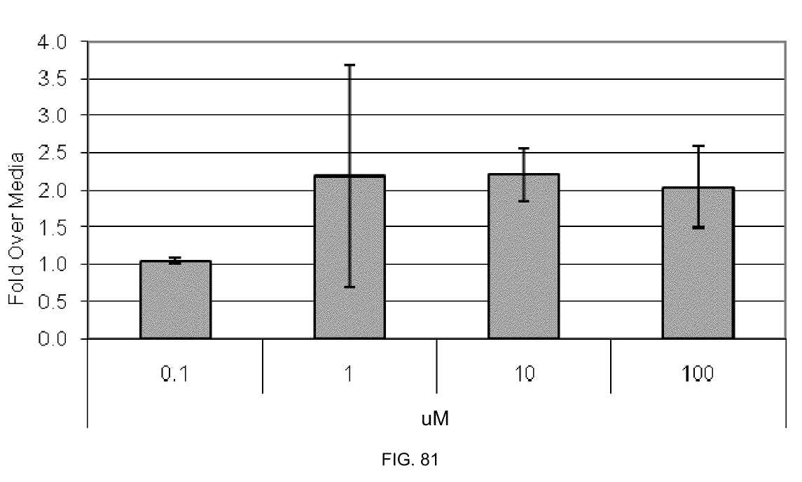

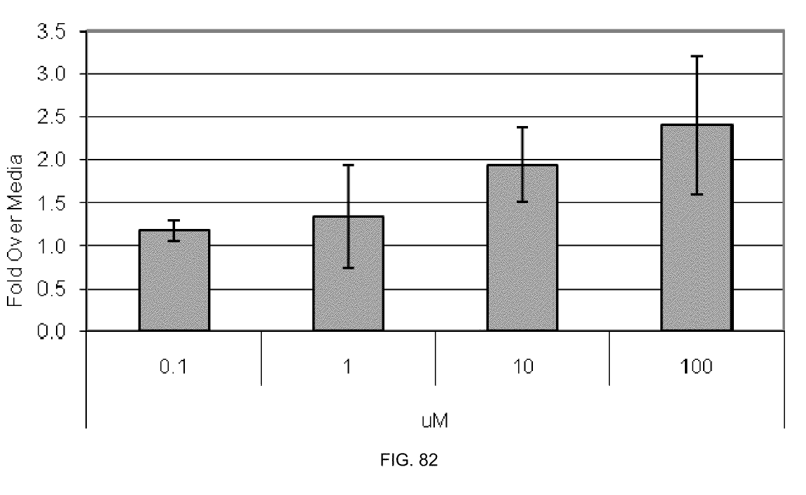

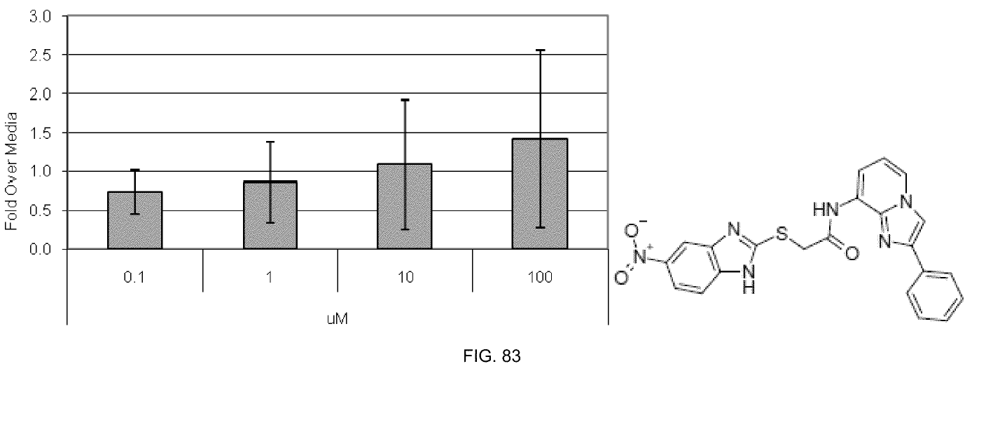

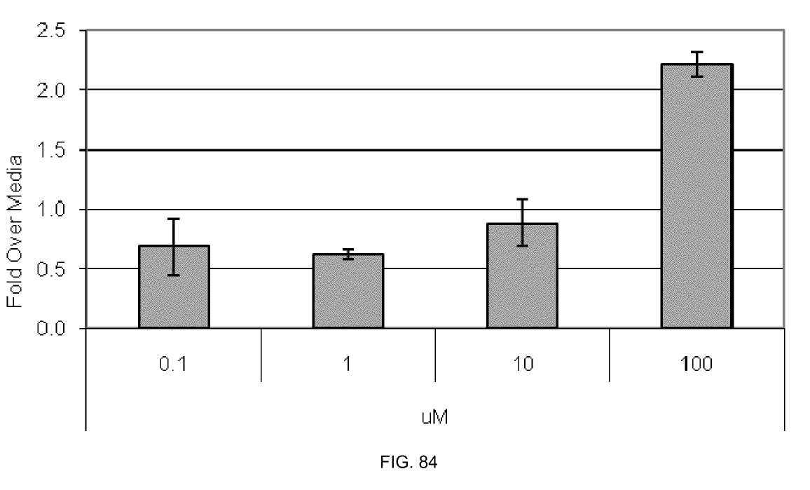

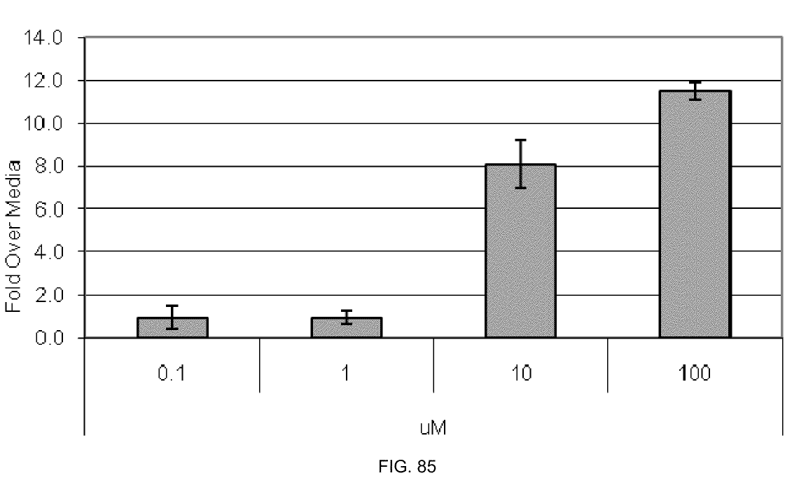

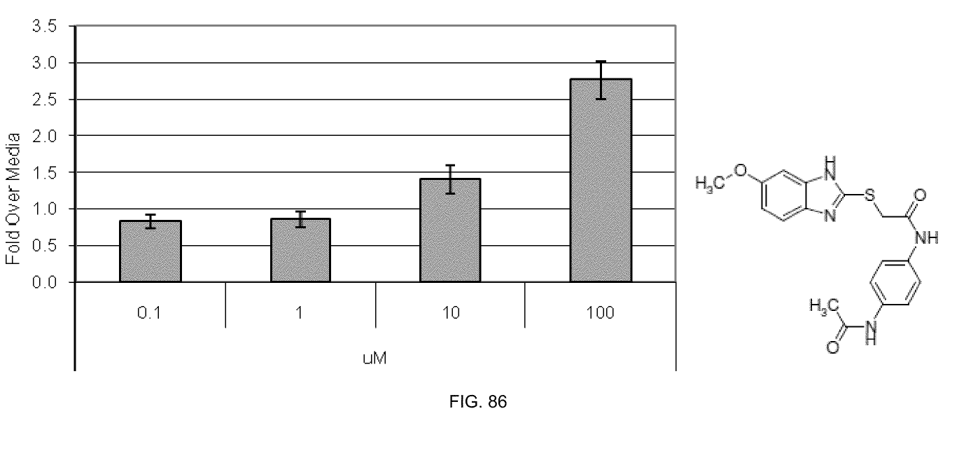

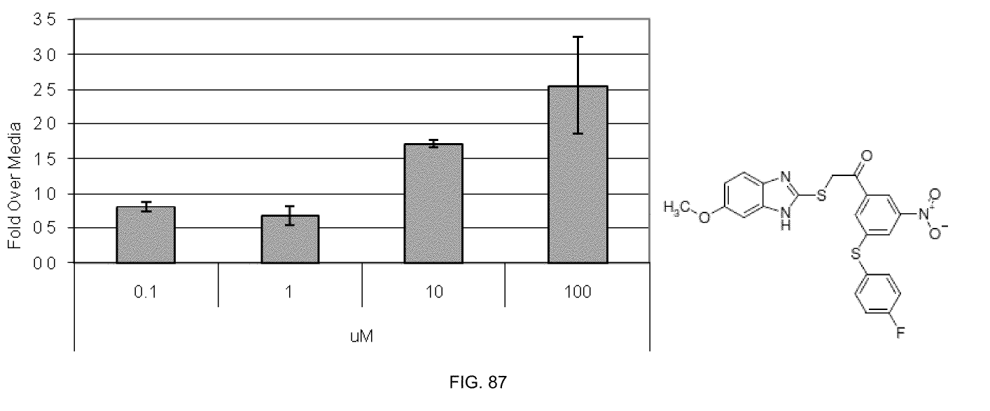

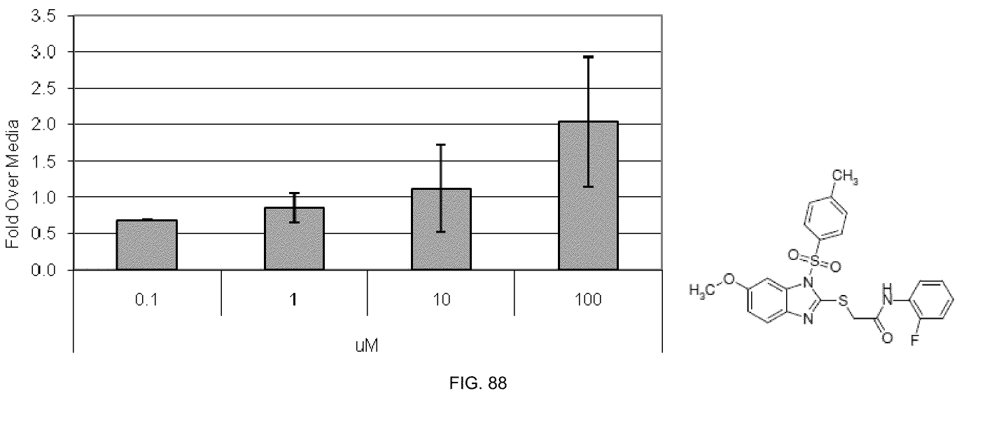

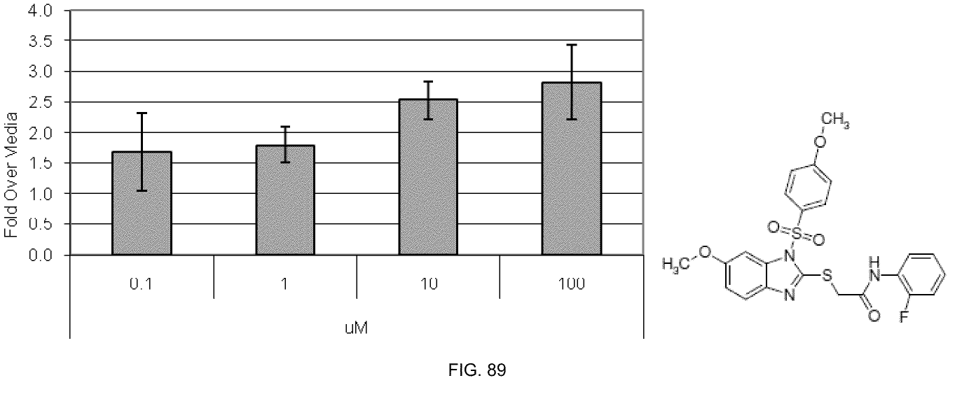

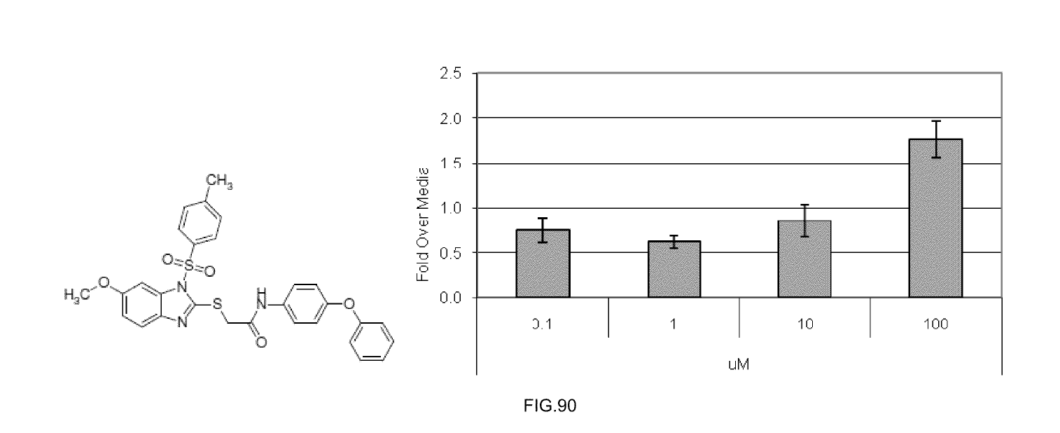

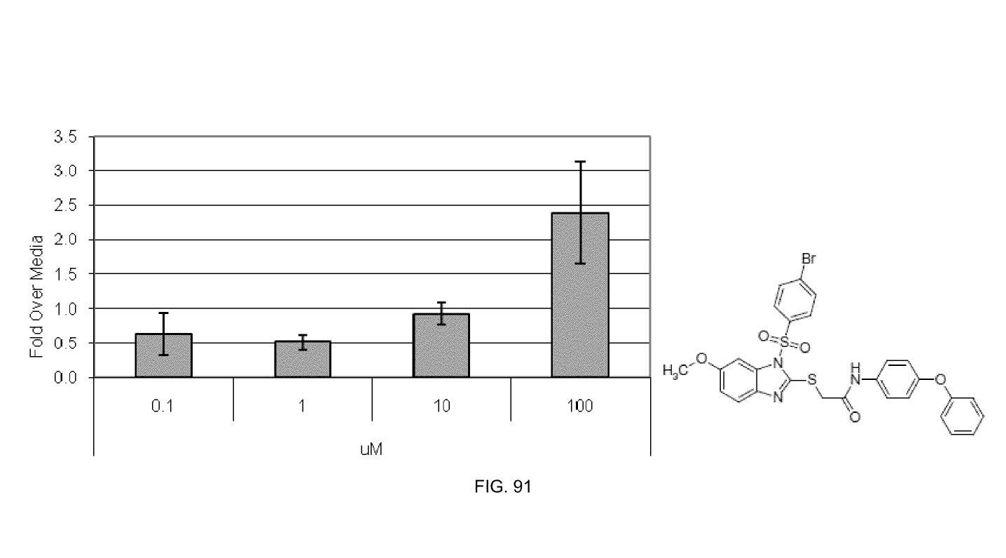

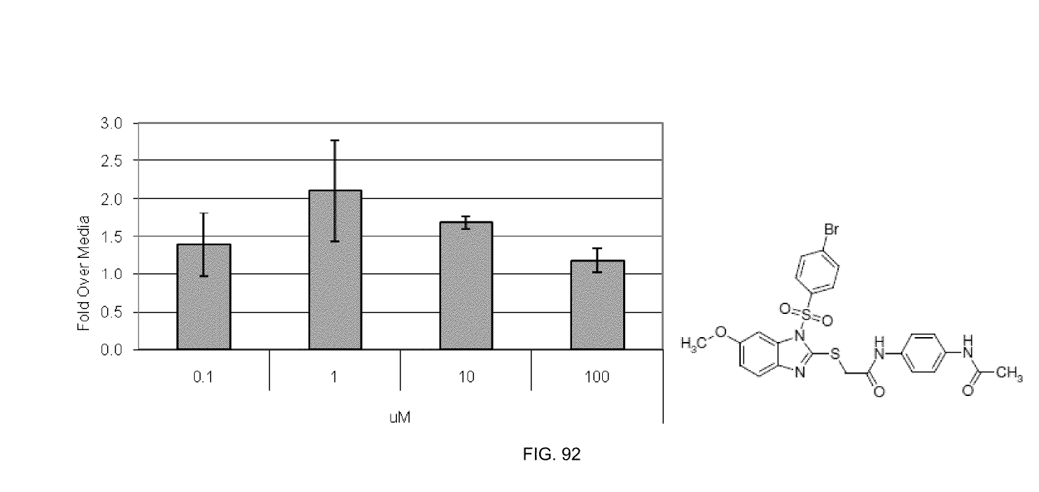

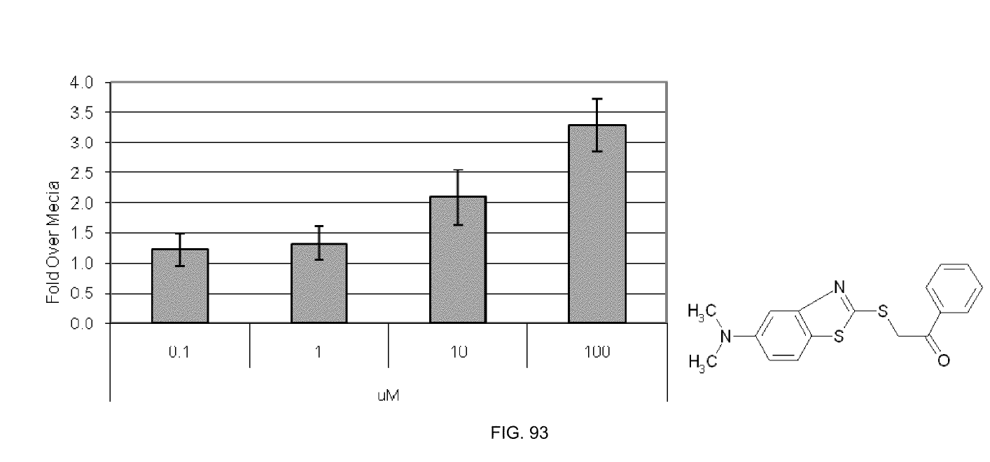

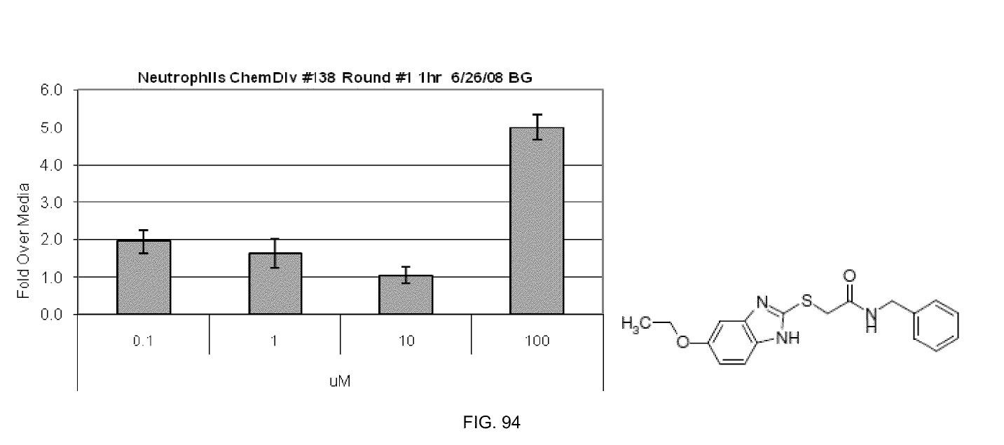

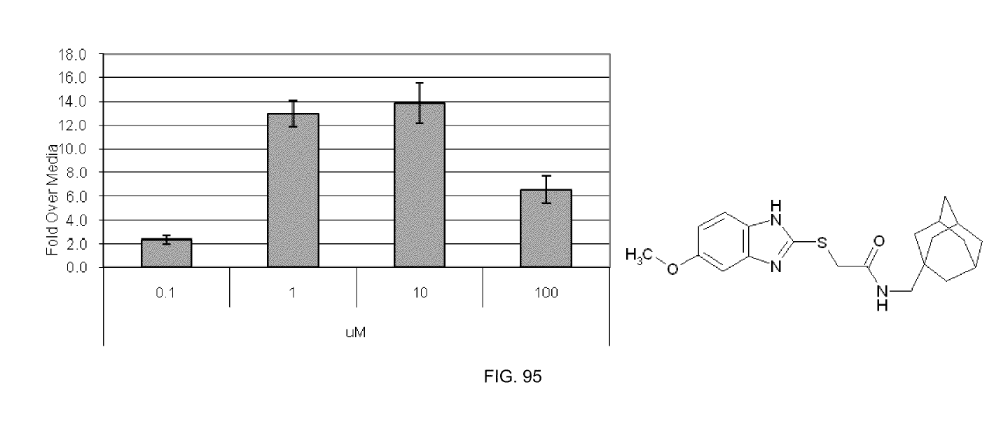

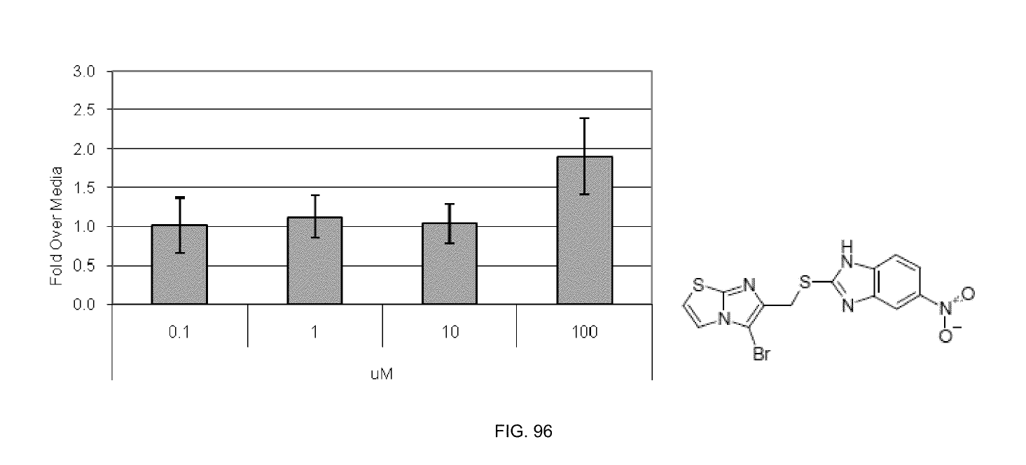

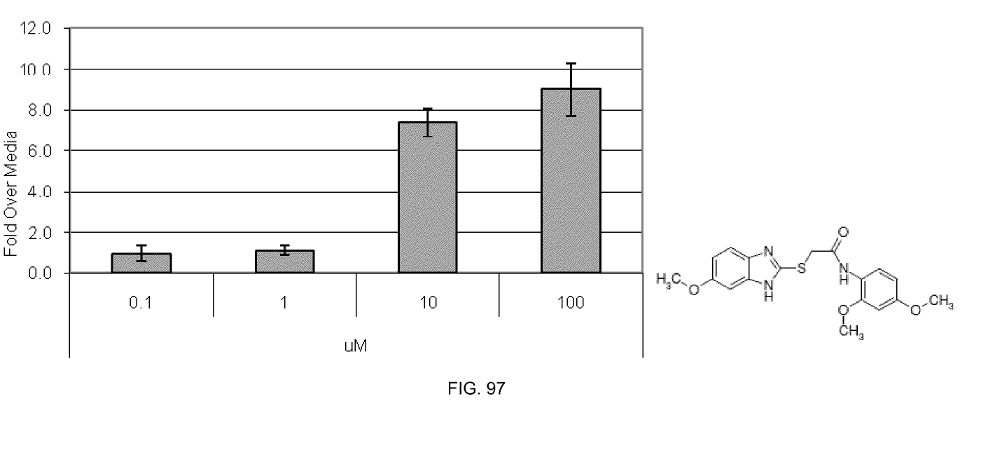

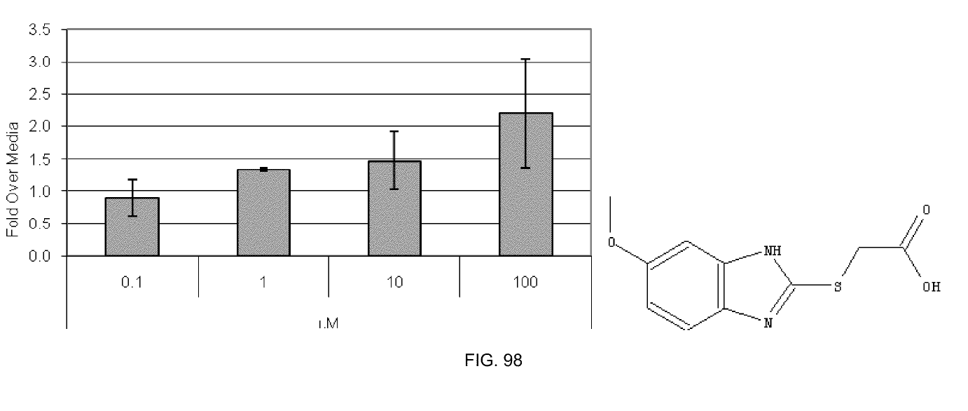

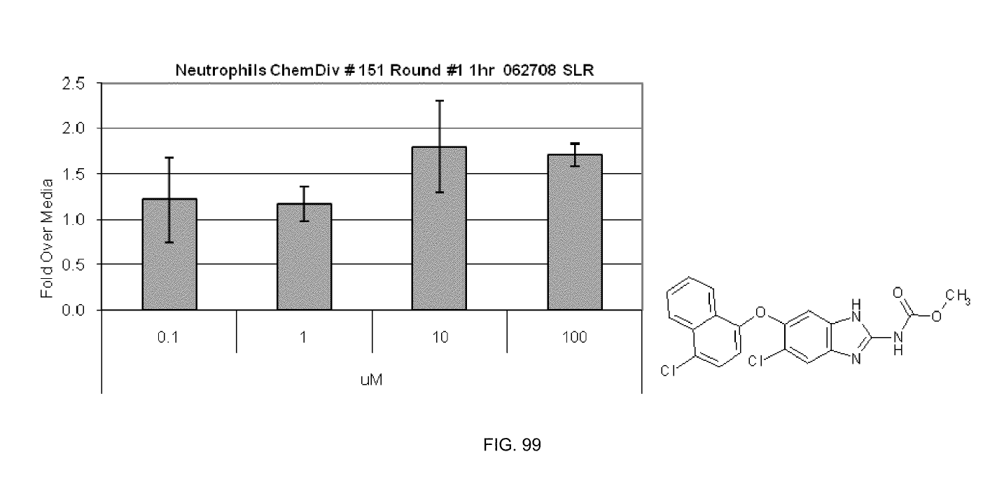

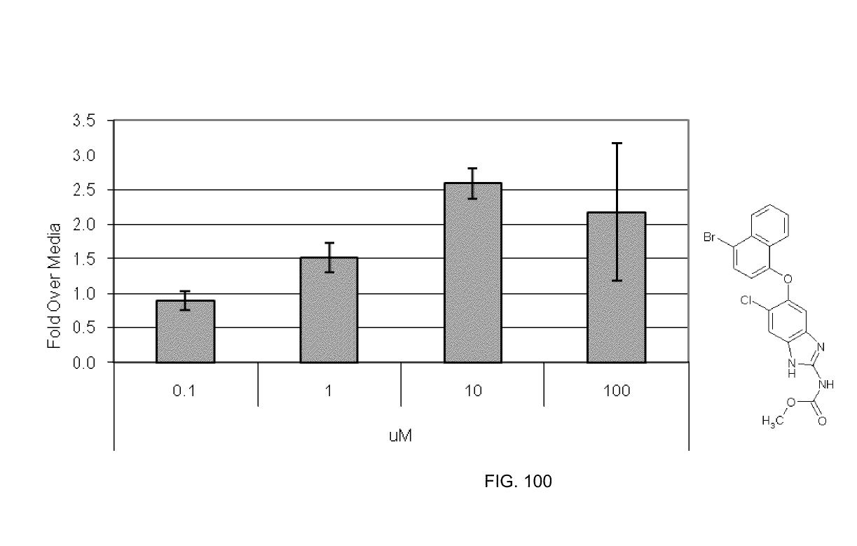

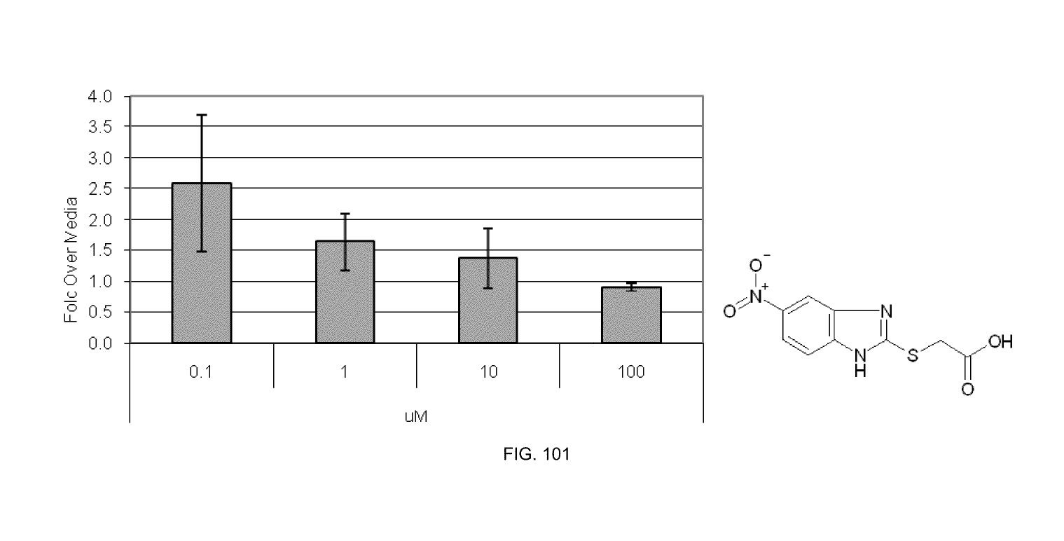

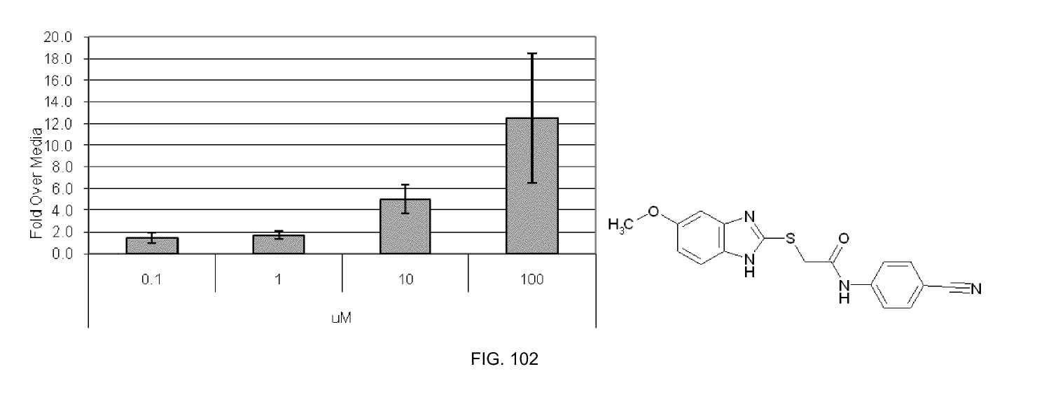

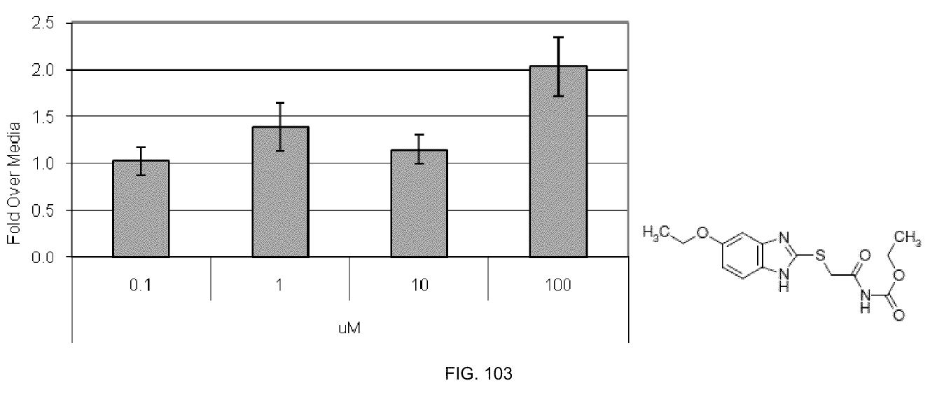

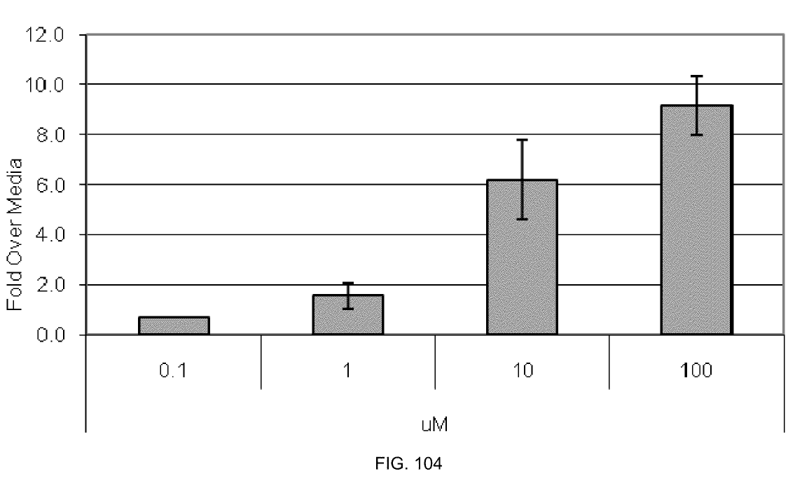

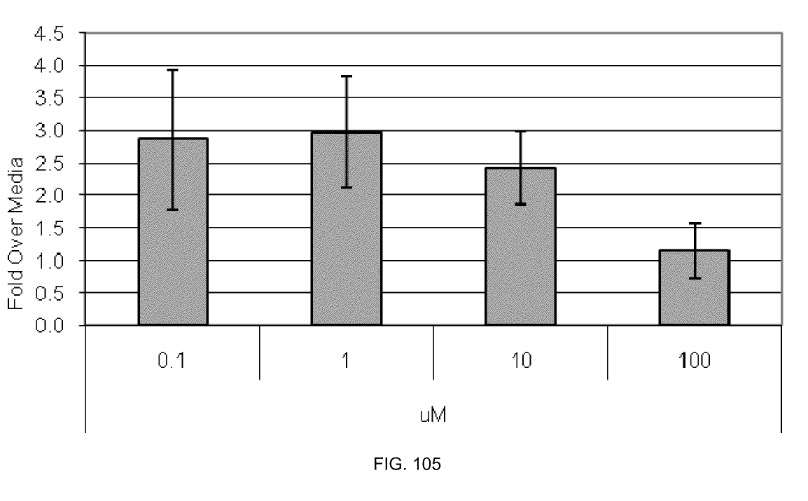

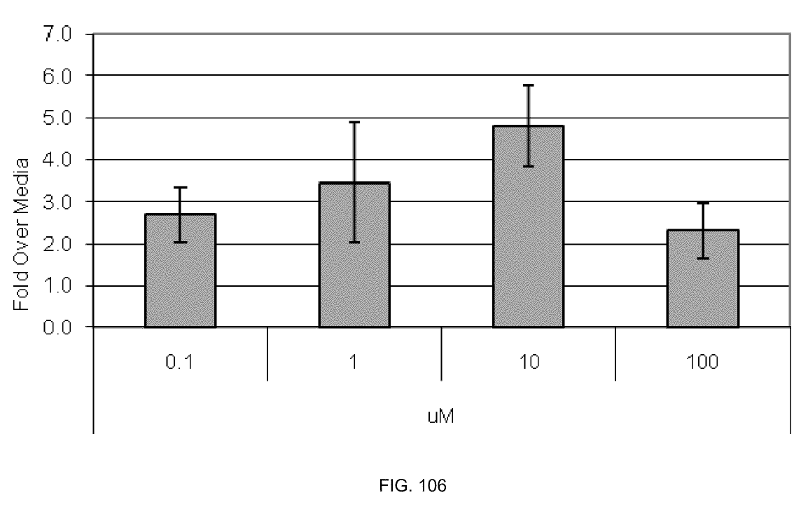

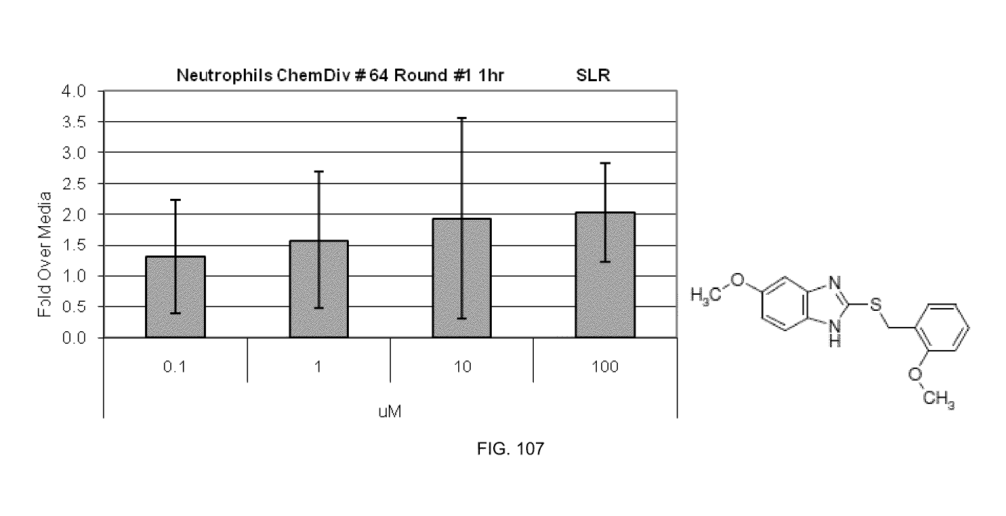

As shown in FIGS. 3-58 and 60-106, cells treated with Compounds 60, 79, 80, 81, 102, 109, 110, 111, 112, 11, 12, 15, 16, 17, 20, 21, 22, 23, 24, 33, 36, 37, 38, 39, 40, 41, 42, 43, 45, 54, 56, 58, 59, 62, 63, 69, 70, 71, 83, 84, 88, 89, 91, 92, 93, 96, 97, 98, 99, 100, 101, 102, 107, 128, 133, 134, 136, 137, 138, 139, 154, 159, 160, 157, 142, 143, 144, 132, and 135 (shown above in Tables 1-4 and labeled with a “*”) showed at least 3-fold greater chemorepulsion than when treated with media.

In this experiment, neutrophils treated with the Compounds 4, 5, 6, 7, 9, 10, 18, 19, 26, 27, 30, 32, 35, 46, 48, 49, 50, 52, 53, 55, 57, 61, 64, 65, 67, 68, 73, 74, 75, 76, 77, 82, 85, 90, 94, 95, 106, 108, 113, 114, 115, 116, 117, 118, 119, 120, 121, 122, 123, 124, 125, 140, 141, 148, 149, 151, 152, 153, 155, 156, 158, 87, 104, 34, 72, 126, 127, 147, 44, 2, 25, 28, 29 and 47 showed chemorepulsion that was less than 3-fold that seen in neutrophils treated with media alone.

### Example 5

### Rabeprazole Induces Negative Induction of Human Neutrophils

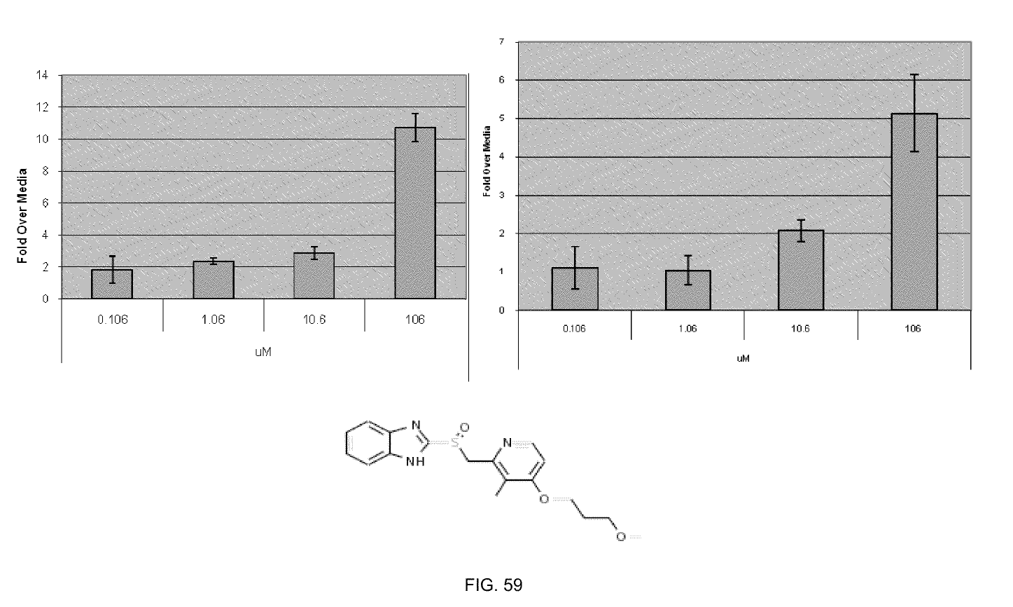

As shown in FIG. 59, cells treated with rabeprazole at 106 uM showed greater than 3-fold chemorepulsion over media.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inducing negative chemotaxis of a human migratory cell comprising administering an effective amount of a compound having a structure selected from the group consisting of: 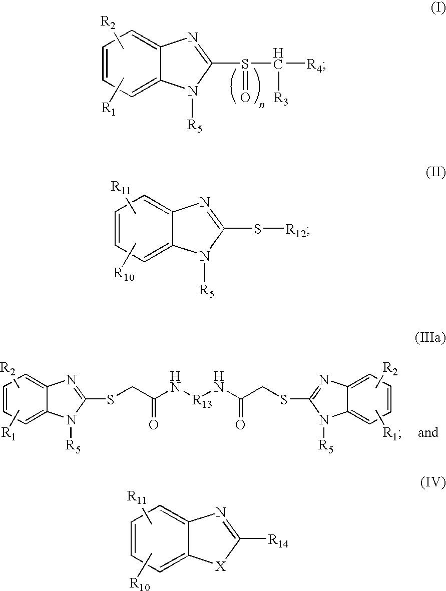
or a pharmaceutically acceptable salt thereof;
wherein:
R₁ and R₂ are the same or different and each are selected from the group consisting of hydrogen, C1-C10 alkyl, halo, OR₉, C(O)R₉, C(O)OR₉, NO₂ and NR₆R_{6′};
R₃ is selected from the group consisting of H, C1-C10 alkyl and aryl;
R₄ is selected from the group consisting of C1-C10 alkyl, C1-C10 alkyl substituted with one or more R₇, C(O)R₆, C(O)OR₆, C(O)NR₆R_{6′}, benzyl, aryl and heteroaryl, wherein said aryl and heteroaryl are each optionally substituted with one or more R₈;
Each R₅ is independently selected from the group consisting of hydrogen, C1-C10 alkyl and S(O)ₚR₆;
R₆ and R₆′ are each independently selected from the group consisting of hydrogen, C1-C10 alkyl, C1-C10 alkyl substituted with one or more R₇, C2-C10 alkeynyl, C2-C10 alkenyl substituted with one or more R₇, halo, OR₉, cycloalkyl, cycloalkyl substituted with one or more R₈ bicycloalkyl, bicycloalkyl substituted with one or more R₈, heterocylic, heterocyclic substituted with one or more R₈, C(O)R₉, OC(O)R₉, C(O)OR₉, benzyl, aryl and heteroaryl, wherein said aryl and heteroaryl are each optionally substituted with one or more R₈, or wherein R₆ and R_{6′} are taken together with the nitrogen to which they are attached to form a 3-6 membered heterocyclic ring, wherein the ring is optionally substituted with one or more R₈;
Each R₇ is independently selected from the group consisting of halo, NR₁₆R_{16′}, NO₂, C(O)R₉, C(O)OR₉, C(O)NR₁₆R_{16′}, OR₉, S(O)ₚR₁₆, S(O)ₚNR₁₆R_{16′}, SR₁₆, CN, oxo, 5-6 membered heterocyclic ring comprising one or more heteroatoms selected from N, O or S, wherein said heterocyclic ring is optionally substituted with R₉, optionally substituted aryl, optionally substituted heteroaryl, and 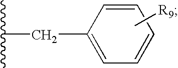
Each R₈ is independently selected from the group consisting of R₇, C1-C10 alkyl and C1-C10 alkyl substituted with one or more R₇;
Each R₉ is independently selected from the group consisting of hydrogen, optionally substituted aryl, optionally substituted heteroaryl, C1-C10 alkyl, C1-C10 alkyl substituted with one or more group selected from halo, optionally substituted aryl and optionally substituted heteroaryl;
R₁₀ and R₁₁ are the same or different and are each independently selected from the group consisting of hydrogen, C1-C10 alkyl, halo, OR₉, C(O)R₉, C(O)OR₉, NO₂ and NR₆R_{6′}, SR₆, aryl, heteroaryl, wherein each of said aryl and heteroaryl is optionally substituted with one or more R₈, and 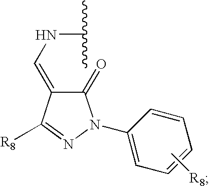
R₁₂ is selected from the group consisting of hydrogen, C1-C10 alkyl, aryl, heteroaryl, wherein said each of said aryl and heteroaryl are optionally substituted by one or more R₈;
R₁₃ is selected from the group consisting of: 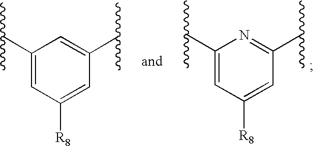
R₁₄ is selected from the group consisting of hydrogen, C1-C10 alkyl, C1-C10 alkyl substituted with one or more R₇, S(O)₂OR₆, NR₆R_{6′}, SR₆, OR₆, aryl, and heteroaryl, wherein each of said aryl and heteroaryl each optionally substituted with one or more R₈, and 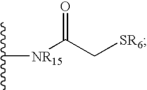
X is selected from the group consisting of O, S and NR₁₅;
R₁₅ is hydrogen or C1-C10 alkyl;
R₁₆ and R₁₆′ are each independently selected from the group consisting of hydrogen, C1-C10 alkyl, C1-C10 alkyl substituted with one or more R₉, C2-C10 alkeynyl, C2-C10 alkenyl substituted with one or more R₉, halo, OR₉, cycloalkyl, cycloalkyl substituted with one or more R₉, bicycloalkyl, bicycloalkyl substituted with one or more R₉, heterocylic, heterocyclic substituted with one or more R₉, C(O)R₉, OC(O)R₉, C(O)OR₉, benzyl, optionally substituted aryl and optionally substituted heteroaryl; or R₁₆ and R_{16′} are taken together with the nitrogen atom to which they are attached to form a 3-6 membered heterocyclic ring, wherein the ring is optionally substituted with one or more R₉;
n is 0 or 1; and
each p is independently 1 or 2.

2. The method of claim 1, wherein the compound has the Formula (I).

3. The method of claim 2, wherein n is 1.

4. The method of claim 3, wherein R₁ and R₂ are each independently selected from the group consisting of H, C1-C4 alkyl and OR₉, wherein R₉ is a C1-C4 alkyl.

5. The method of claim 4, wherein R₄ is heteroaryl optionally substituted with one or more R₈.

6. The method of claim 5, wherein R₄ is pyridinyl optionally substituted with one or more R₈.

7. The method of claim 6, wherein R₈ is selected from the group consisting of C1-C4 alkyl and OR₉, wherein R₉ is C1-C4 alkyl.

8. The method of claim 2, wherein the compound is selected from the group consisting of omeprazole, esomperazole, lansoprazole, rabeprazole, leminoprazole and pantoprazole.

9. The method of claim 2, wherein the compound has the Formula (Ia):

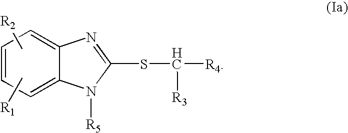

10. The method of claim 9, wherein R₃ is hydrogen.

11. The method of claim 10, wherein R₁ and R₂ are each independently selected from the group consisting of H, OR₉ and NO₂.

12. The method of claim 11, wherein R₄ is selected from the group consisting of C1-C10 alkyl, C1-C10 alkyl substituted with one or more R₇, C(O)R₆, C(O)NR₆R_{6′}, aryl and heteroaryl, wherein said aryl and heteroaryl are each optionally substituted with one or more R₈.

13. The method of claim 9, wherein the compound is selected from the group consisting of compounds having the following formulae:

| | R₁ | R₂ | R₃ | R₅ | R₄ |
|---|---|---|---|---|---|
| 60 | H | OEt | H | H | 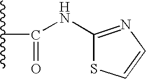 |
| 79 | H | OCH₃ | H | H | 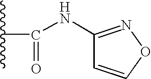 |
| 80 | H | OCH₃ | H | H | 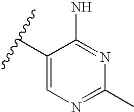 |
| 81 | H | OCH₃ | H | H | 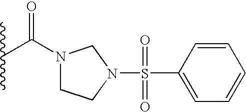 |
| 11 | H | OCH₃ | H | H | 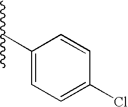 |
| 12 | H | NO₂ | H | H | 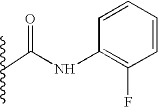 |
| 15 | H | OCH₃ | H | H | 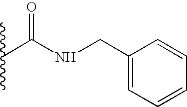 |
| 16 | H | OCH₃ | H | H | 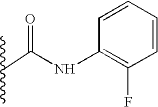 |
| 17 | H | OCH₃ | H | H | 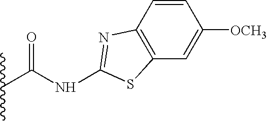 |
| 20 | H | OCH₃ | H | H | 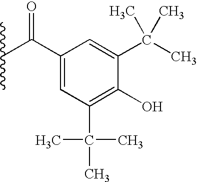 |
| 21 | H | OCH₃ | H | H | 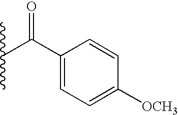 |
| 22 | H | OCH₃ | H | H | 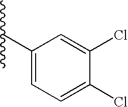 |
| 23 | H | OCH₃ | H | H | 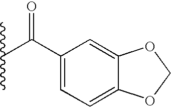 |
| 102 | H | OCH₃ | H | H | 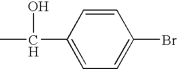 |
| 24 | H | OCH₃ | H | H | 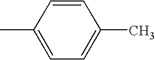 |
| 33 | H | NO₂ | H | H | 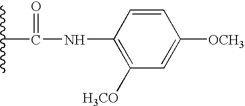 |
| 36 | H | OCH₃ | H | H | 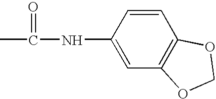 |
| 37 | H | OCH₃ | H | H | 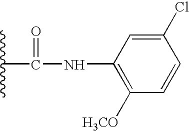 |
| 38 | H | OCH₃ | H | H | 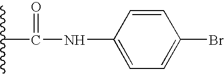 |
| 39 | H | OCH₃ | H | H | 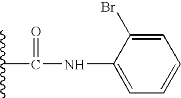 |
| 40 | H | OCH₃ | H | H | 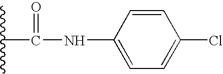 |
| 41 | H | OCH₃ | H | H | 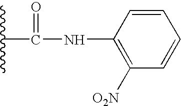 |
| 42 | H | OCH₃ | H | H | 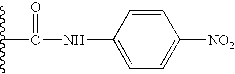 |
| 45 | H | OCH₃ | H | H | 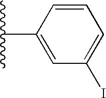 |
| 56 | H | OEt | H | H | 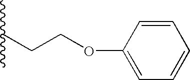 |
| 58 | H | OEt | H | H | 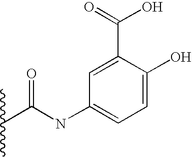 |
| 59 | H | NO₂ | H | H | 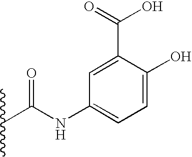 |
| 62 | H | OEt | H | H | 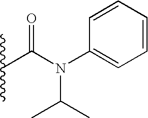 |
| 63 | H | OEt | H | H | 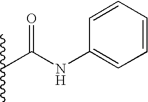 |
| 109 | OCH₃ | H | H | H | 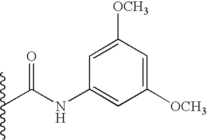 |
| 110 | OCH₃ | H | H | H | 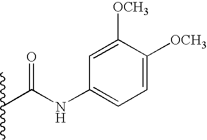 |
| 111 | OCH₃ | H | H | H | 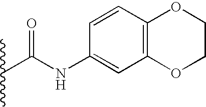 |
| 112 | OCH₃ | H | H | H | 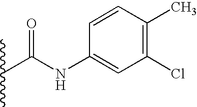 |
| 69 | OCH₃ | H | H | H | 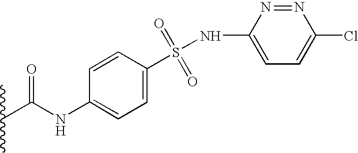 |
| 70 | OCH₃ | H | H | H | 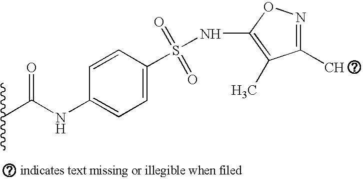 |
| 71 | H | OEt | H | H | 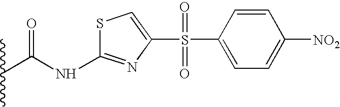 |
| 84 | H | OCH₃ | H | H | 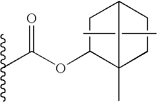 |
| 89 | H | OCH₃ | H | H | 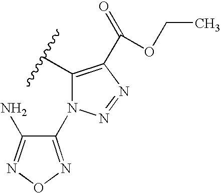 |
| 91 | H | OEt | H | H | 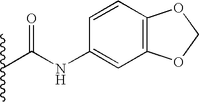 |
| 92 | H | OEt | H | H | 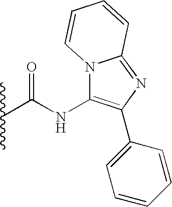 |
| 93 | H | OEt | H | H | 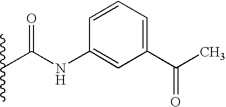 |
| 96 | H | NO₂ | H | H | 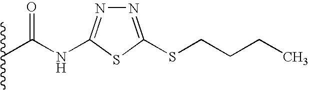 |
| 97 | H | NO₂ | H | H | 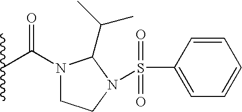 |
| 98 | H | OEt | H | H | 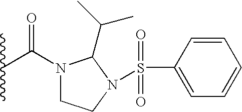 |
| 100 | H | OCH₃ | H | H | 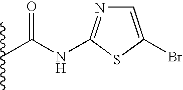 |
| 101 | H | OEt | Ph | H | 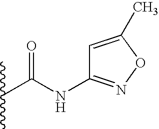 |
| 107 | OCH₃ | H | H | H | 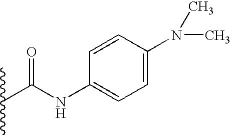 |
| 133 | H | OCH₃ | H | H | 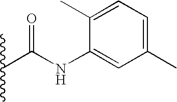 |
| 134 | H | OCH₃ | H | H | 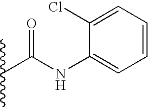 |
| 137 | H | N-(Me)₂ | H | H | 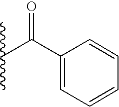 |
| 138 | H | OEt | H | H | 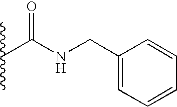 |
| 139 | H | OCH₃ | H | H | 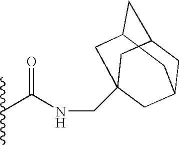 |
| 159 | H | OEt | H | H | 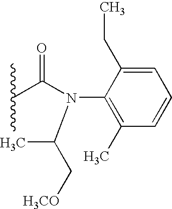 |
| 160 | H | OEt | H | H | 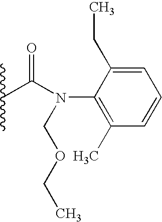 |
| 154 | H | OCH₃ | H | H | 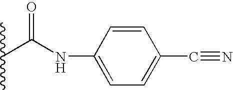 |
| 142 | OCH₃ | H | H | H | 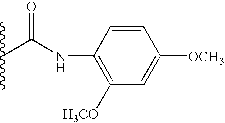 |
| 143 | OCH₃ | H | H | H | 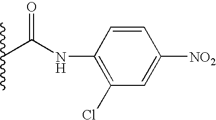 |
| 144 | OCH₃ | H | H | H | 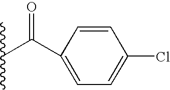 |

14. The method of claim 1, wherein the compound has the Formula (II).

15. The method of claim 14, wherein the compound has the Formula (IIa):

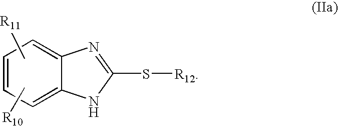

16. The method of claim 15, wherein R₁₀ and R₁₁ are each independently selected from the group consisting of hydrogen, OR₉ and halo.

17. The method of claim 16, wherein R₁₂ is selected from the group consisting of C1-C10 alkyl, aryl and heteroaryl, wherein said each of said aryl and heteroaryl are optionally substituted by one or more R₈.

18. The method of claim 15, wherein the compound is selected from the group consisting of compounds having the following formulae:

| | R₁₀ | R₁₁ | R₁₂ |
|---|---|---|---|
| 54 | H | OCH₃ | 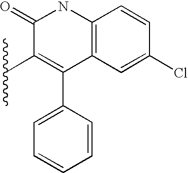 |
| 88 | H | OCH₃ | 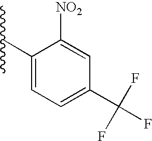 |
| 99 | H | OCH₃ | 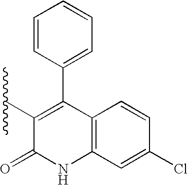 |
| 136 | 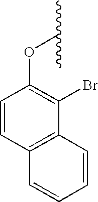 | Cl | CH₃ |
| 135 | OCH₃ | H | 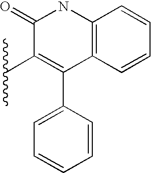 |

19. The method of claim 1, wherein the compound has the Formula (IIIa).

20. The method of claim 19, wherein R₁ is hydrogen, R₂ is methoxy, R₅ is hydrogen;
and R₁₃ is

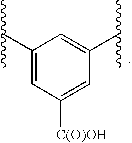

21. The method of claim 1, wherein the compound has the Formula (IV).

22. The method of claim 20, wherein the compound has the Formula (IV), wherein X is selected from the group consisting of NR₅ and S.

23. The method of claim 20, wherein X is selected from the group consisting of NR₅, wherein R₅ is hydrogen or C1-C4 alkyl.

24. The method of claim 20, wherein the R₁₀ and R₁₁ are each independently selected from the group consisting of hydrogen, OR₉ and NO₂.

25. The method of claim 22, wherein R₁₄ is NR₆R_{6′}, wherein R₆ and R_{6′} are taken together to form a 3-6 membered ring, wherein the ring is optionally substituted with one or more R₈.

26. The method of claim 22, wherein R₁₄ is heteroaryl.

27. The method of claim 20, wherein the compound is selected from the group consisting of compounds having the following formulae:

| | X | R₁₀ | R₁₁ | R₁₄ |
|---|---|---|---|---|
| 128 | NH | H | OCH₃ | 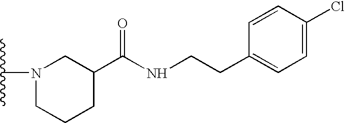 |
| 132 | S | H | NO₂ | 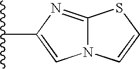 |
| 157 | NH | H | OCH₃ | 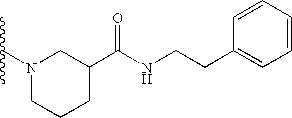 |
| 83 | NH | H | S(CH₂)₂CH₃ | 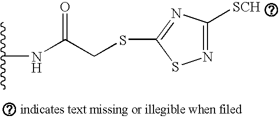 |

28. The method of claim 1, wherein the human migratory cell is an immune cell.

29. The method of claim 8, wherein the compound is selected from the group consisting omeprazole, esomeprazole and rabeprazole.

30. The method of claim 1, wherein the negative chemotaxis of an immune cell is induced in a patient in need thereof, wherein the compound is administered in a therapeutically effective amount.

31. The method of claim 30, wherein the patient is suffering from an inflammatory condition and the compound is administered in amount sufficient to treat the inflammatory condition.

32. The method of claim 30, wherein the compound is administered locally to the site of inflammation.

33. The method of claim 31, wherein the inflammatory condition is injection site reaction.

34. The method of claim 1, wherein angiogenesis is inhibited in a patient in need thereof and wherein the patient is suffering from cancer or a tumor.

35. The method of claim 1 wherein negative chemotaxis of a cell to a medical implant or to a transplant or a graft is induced in a patient in need thereof.

36. The method of claim 35, wherein the compound is administered by coating a surface of the medical implant.

37. The method of claim 35, wherein the compound is administered locally at the site of the medical implant.
